(19) United States
(12) Patent Application Publication
(10) Pub. No.: US 2023/0174640 A1
(43) Pub. Date: Jun. 8, 2023

(54) Use of Anti-IL-6 Antibody, e.g., Clazakizumab for Treatment/Prevention of ARDS Associated with Coronavirus (COVID-19) Infection

(71) Applicant: Vitaeris Inc., Vancouver (CA)

(72) Inventors: Kevin Chow, Sammamish, WA (US); Edward Chong, North Vancouver (CA); Bonnie Lonze, New York, NY (US); Robert Montgomery, Parkton, MD (US)

(73) Assignee: Vitaeris Inc., Vancouver, BC (CA)

(21) Appl. No.: 17/911,808

(22) PCT Filed: Mar. 17, 2021
(86) PCT No.: PCT/US2021/022838

§ 371 Date: Sep. 15, 2022

Related U.S. Application Data
(60) Provisional application No. 63/152,612, filed on Feb. 23, 2021.
(60) Provisional application No. 63/018,681, filed on May 1, 2020.
(60) Provisional application No. 62/994,311, filed on Mar. 25, 2020.
(60) Provisional application No. 62/993,765, filed on Mar. 24, 2020.
(60) Provisional application No. 62/991,270, filed on Mar. 18, 2020.

(51) Int. Cl. C07K 16/24; A61P 31/14

(52) U.S. Cl. CPC C07K 16/248; A61P 31/14; C07K 2317/565; C07K 2317/24

(57) ABSTRACT

The present disclosure relates to uses of an anti-IL-6 antibody, e.g., clazakizumab in order to prevent, stabilize, reduce, or otherwise treat acute or chronic respiratory distress syndrome (ARDS or CRDS) and symptoms thereof such as lung damage, and also to treat cytokine storm syndrome, in patients with or suspected of having a bacterial, viral, or fungal infection, such as a coronavirus infection, e.g., COVID-19, SARS, MERS, or another bacterial or viral infection which may cause acute or chronic respiratory distress syndrome or cytokine storm syndrome.

### CROSS-REFERENCE TO RELATED APPLICATIONS

This application is a national phase entry pursuant to 35 U.S.C. § 371 of International Application No. PCT/US2021/022838, filed Mar. 17, 2021, which claims priority from U.S. Application No. 63/152,612, filed Feb. 23, 2021, U.S. Application No. 63/018,681, filed May 1, 2020, U.S. Application No. 62/994,311, filed Mar. 25, 2020, U.S. Application No. 62/993,765, filed Mar. 24, 2020, and U.S. Application No. 62/991,270, filed Mar. 18, 2020, the entire contents of which are incorporated by reference herein for all purposes.

### SEQUENCE LISTING

This disclosure contains a sequence listing containing sequences of exemplary anti-IL-6 antibodies suitable for use in the claimed therapies, entitled “2021-03-17_WO_Sequence Listing_ST25”, created Aug. 18, 2022, having a file size of 34000 Bytes, which is incorporated by reference herein.

### FIELD

The present disclosure relates to uses of an anti-human IL-6 antibody, e.g., clazakizumab in order to treat (e.g., by preventing, stabilizing, or reducing) acute or chronic respiratory distress syndrome (ARDS or CRDS) and symptoms thereof such as lung damage, and also to treat cytokine storm syndrome in patients with or suspected of having a bacterial, viral, or fungal infection, such as a coronavirus infection, e.g., COVID-19, SARS, MERS, or another bacterial or viral infection which may cause acute or chronic respiratory distress syndrome. In exemplary embodiments the methods are used in patients known or suspected to be infected with COVID-19, or another infectious agent, wherein optionally the patient may already exhibit signs of lung damage and/or or pneumonia and/or may be on a respirator or ventilator. The foregoing treatments may be provided in combination with one or more other treatments for any of acute or chronic respiratory distress syndrome, pneumonia and/or viral or bacterial infection such as steroids, anti-virals and antibiotics.

### BACKGROUND

Interleukin-6 (IL-6) plays pathologic roles in immune-inflammatory diseases such as rheumatoid arthritis (RA) and Castleman disease. By inhibiting IL-6 clazakizumab (Claza) (a humanized anti-IL-6 antibody) ameliorates the symptoms of these diseases and normalize acute-phase proteins, including C-reactive protein (CRP).

Some patients infected with viruses or bacteria including COVID-19 can develop an uncontrolled immune response, leading to potentially life-threatening damage to lung tissue. In particular, in some subjects with COVID-19 infection or other bacterial, viral, or fungal infections, the body may respond to the pathogen by overproducing immune cells and their signaling molecules including IL-6 in a dangerous phenomenon called cytokine storm. New and improved treatments for ARDS and CRDS and cytokine storm syndrome and side effects thereof such as lung damage caused thereby, especially that caused by coronavirus infections such as COVID-19 infection are urgently needed.

### BRIEF DESCRIPTION OF THE FIGURES

FIG. 1 shows serial measurements of serum CRP in seven patients in the days following administration of the first dose of clazakizumab (25 mg intravenously). Two patients received a second 25 mg dose within 24-48 hrs of the first dose.

FIG. 2 provides a schematic of the study design described in Example 5 below.

FIG. 3 provides a schematic of the study design described in Example 6 below.

FIG. 4 provides a schematic of the study design described in Example 7 below. Specifically, 81 patients were enrolled in the phase 2 dose-finding portion of the trial beginning on Apr. 1, 2020. On May 3, 2020, the low-dose clazakizumab arm was dropped for lack of efficacy and the 24 patients who received low-dose were excluded from efficacy analyses. 97 additional patients were enrolled in the phase 3 portion and were randomized 1:1 (high-dose clazakizmab: placebo). The efficacy analyses were based on data collected from 78 patients who received high-dose clazakizumab and 72 patients who received placebo.

FIGS. 5A-5D show Bayesian models of primary and secondary outcomes from the study described in Example 7 below. For the primary outcome of 28-day ventilator free survival (FIG. 5A) and for overall 28-day survival (FIG. 5B), curves illustrate the estimated posterior distribution of the odds ratio comparing clazakizumab to placebo. Odds ratios greater than 1 (shaded more lightly, on the right side of the curve) indicate a benefit of clazakizumab compared to placebo. Vertical lines indicate the reference values for the odds ratios of 1.0 (no benefit of clazakizumab) and 1.25 (meaningful clinical benefit of clazakizumab). For the secondary outcomes of 14-day (FIG. 5C) and 28-day (FIG. 5D) ordinal clinical scores, curves again illustrate the estimated posterior distribution of odds ratios comparing clazakizumab to placebo. Here, a lower ordinal score correlates with a more favorable clinical status, and odds ratios less than 1 (shaded more lightly, on the left side of the curve) indicate a benefit of clazakizumab compared to placebo. Vertical lines indicate the reference values for the odds ratios of 1.0 (no benefit of clazakizumab) and 0.8 (meaningful clinical benefit of clazakizumab). 95% credible intervals are depicted in the inset tables, along with the posterior probabilities of the odds ratios exceeding the reference values.

### SUMMARY

This disclosure relates in part to the use of an anti-IL-6 monoclonal antibody (mAb), e.g., clazakizumab for the treatment of acute respiratory distress syndrome (ARDS) or chronic respiratory distress syndrome (CRDS) and the amelioration of side effects associated therewith such as lung damage and cytokine storm syndrome in patients infected with or suspected of being infected with a virus or bacteria which causes acute or chronic respiratory distress syndrome, e.g., a coronavirus such as COVID-19.

Clazakizumab comprises the heavy and light chain sequences set forth below:

(Heavy chain)
SEQ ID NO: 745
EVQLVESGGGLVQPGGSLRLSCAASGFSLSNYYVTWVRQAPGKGLEWVGI
IYGSDETAYATSAIGRFTISRDNSKNTLYLQMNSLRAEDTAVYYCARDDS
SDWDAKFNLWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKRVEPKSCDKTHTCPPCPAPELLGGPSVFLFPPKP
KDTLMISRTPEVTCVVVDVSHEDPEVKFNWYVDGVEVHNAKTKPREEQYA
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
(Light chain)
SEQ ID NO: 746
AIQMTQSPSSLSASVGDRVTITCQASQSINNELSWYQQKPGKAPKLLIYR
ASTLASGVPSRFSGSGSGTDFTLTISSLQPDDFATYYCQQGYSLRNIDNA
FGGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEVT
HQGLSSPVTKSFNRGEC

It is another specific object of the disclosure to provide therapeutic protocols which include the administration or use of an anti-IL-6 antibody for treating acute or chronic respiratory distress syndrome and the amelioration of side effects associated therewith such as lung damage and cytokine storm in patients infected or suspected of being infected by COVID-19, optionally those who are demonstrated to express elevated IL-6 levels.

It is another specific object of the disclosure to provide therapeutic protocols which include the administration or use of an anti-IL-6 antibody for treating respiratory distress syndrome and the amelioration of side effects associated therewith such as lung damage and cytokine storm in patients infected or suspected of being infected by COVID-19 who exhibit signs of lung damage including severe lung damage including those on a ventilator or respirator.

It is another specific object of the disclosure to provide therapeutic protocols which include the administration or use of an anti-IL-6 antibody for treating respiratory distress syndrome and the amelioration of side effects associated therewith such as lung damage and cytokine storm in patients infected or suspected of being infected by COVID-19 who have or are suspected of having pneumonia, e.g., pneumonia caused by COVID-19 or another viral or bacterial pathogen such as an influenza virus, or a viral, bacterial, or fungal infectious agent that causes pneumonia, such as Streptococcus pneumoniae or Mycoplasma pneumoniae.

It is another specific object of the disclosure object to provide therapeutic protocols such as for treating lung damage and cytokine storm syndrome in patients infected or suspected of being infected by COVID-19 or another viral, bacterial, or fungal agent, which include the administration or use of an anti-IL-6 antibody in combination with other therapies for treating respiratory distress syndrome, pneumonia and/or infection such as steroids, antivirals, antibiotics, and the like.

It is another object of the disclosure to provide novel protocols which include the administration or use of an anti-IL-6 antibody for treating respiratory distress syndrome and the amelioration of side effects associated therewith by the use of specific anti-IL-6 antibodies and antibody fragments, e.g., clazakizumab wherein IL-6 and/or CRP levels are detected prior, during or after antibody administration.

It is another specific object of the disclosure to provide therapeutic protocols for treating respiratory distress syndrome and the amelioration of side effects associated therewith such as lung damage and cytokine storm in patients infected or suspected of being infected by COVID-19, comprising administering to said subject a prophylactically or therapeutically effective amount of an anti-human interleukin-6 (IL-6) antibody or anti-IL-6 antibody fragment, wherein the antibody or antibody fragment comprises: a variable light chain polypeptide comprising the CDRs of SEQ ID NOs:4, 5 and 6 and, and a variable heavy chain polypeptide comprising the CDRs of SEQ ID NOs:7, 8 or 120, and 9, e.g., wherein the antibody comprises a V_{H} and V_{L} polypeptide respectively at least 90, 95, 96, 97, 98 or 99% identical to the polypeptides of SEQ ID NO:657 and 709, for example, wherein the antibody is clazakizumab.

It is another specific object of the disclosure to provide therapeutic protocols for treating acute or chronic respiratory distress syndrome and the amelioration of side effects associated therewith such as lung damage and cytokine storm in patients infected or suspected of being infected by COVID-19 by the use of specific anti-IL-6 antibodies and antibody fragments wherein the antibody or antibody fragment comprises: a variable light chain polypeptide comprising the CDRs of SEQ ID NOs:4, 5 and 6 and, and a variable heavy chain polypeptide comprising the CDRs of SEQ ID NOs:7, 8 or 120, and 9, e.g., wherein the antibody comprises a V_{H} and V_{L} polypeptide respectively at least 90, 95, 96, 97, 98 or 99% identical to the polypeptides of SEQ ID NO:657 and 709 and preferably wherein the antibody is clazakizumab, wherein the patient is evaluated prior, during or after treatment to detect whether IL-6 and/or CRP levels are elevated.

It is another specific object of the disclosure to provide therapeutic protocols for treating acute or chronic respiratory distress syndrome and the amelioration of side effects associated therewith such as lung damage and cytokine storm in patients infected or suspected of being infected by COVID-19 by the use of specific anti-IL-6 antibodies and antibody fragments wherein the antibody or antibody fragment comprises: a variable light chain polypeptide comprising the CDRs of SEQ ID NOs:4, 5 and 6 and, and a variable heavy chain polypeptide comprising the CDRs of SEQ ID NOs:7, 8 or 120, and 9, e.g., wherein the antibody comprises a V_{H} and V_{L} polypeptide respectively at least 90, 95, 96, 97, 98 or 99% identical to the polypeptides of SEQ ID NO:657 and 709, for example, wherein the antibody is clazakizumab, in patients in need thereof, e.g., those exhibiting signs of lung damage and/or pneumonia, wherein the treatment further includes the administration of at least one other immunosuppressant, optionally any of thymoglobulin, basiliximab, mycophenolate mofetil, tacrolimus, an anti-CD20 mAb such as rituximab, and corticosteroids.

It is another specific object of the disclosure to provide therapeutic protocols for treating acute or chronic respiratory distress syndrome and the amelioration of side effects associated therewith such as lung damage and cytokine storm in patients infected or suspected of being infected by COVID-19 by the use of specific anti-IL-6 antibodies and antibody fragments wherein the antibody or antibody fragment comprises: a variable light chain polypeptide comprising the CDRs of SEQ ID NOs:4, 5 and 6 and, and a variable heavy chain polypeptide comprising the CDRs of SEQ ID NOs:7, 8 or 120, and 9, e.g., wherein the antibody comprises a V_{H} and V_{L} polypeptide respectively at least 90, 95, 96, 97, 98 or 99% identical to the polypeptides of SEQ ID NO:657 and 709, for example, wherein the antibody is clazakizumab, for example, wherein the antibody is administered intravenously or subcutaneously.

It is another specific object of the disclosure to provide therapeutic protocols for treating acute or chronic respiratory distress syndrome and the amelioration of side effects associated therewith such as lung damage and cytokine storm in patients infected or suspected of being infected by COVID-19 by the use of specific anti-IL-6 antibodies and antibody fragments wherein the antibody or antibody fragment comprises: a variable light chain polypeptide comprising the CDRs of SEQ ID NOs:4, 5 and 6 and, and a variable heavy chain polypeptide comprising the CDRs of SEQ ID NOs:7, 8 or 120, and 9, e.g., wherein the antibody comprises a V_{H} and V_{L} polypeptide respectively at least 90, 95, 96, 97, 98 or 99% identical to the polypeptides of SEQ ID NO:657 and 709, for example, wherein the antibody is clazakizumab, for example, wherein the anti-IL-6 antibody is administered at a dose ranging from about 0.01 mg-5000 mg, more typically from 0.1-1000 mg, and even more typically from 1-500 mg, for example by intravenous or subcutaneous administration.

It is another specific object of the disclosure to provide therapeutic protocols for treating acute or chronic respiratory distress syndrome and the amelioration of side effects associated therewith such as lung damage and cytokine storm in patients infected or suspected of being infected by COVID-19 by the use of specific anti-IL-6 antibodies and antibody fragments wherein the antibody or antibody fragment comprises: a variable light chain polypeptide comprising the CDRs of SEQ ID NOs:4, 5 and 6 and, and a variable heavy chain polypeptide comprising the CDRs of SEQ ID NOs:7, 8 or 120, and 9, e.g., wherein the antibody comprises a V_{H} and V_{L} polypeptide respectively at least 90, 95, 96, 97, 98 or 99% identical to the polypeptides of SEQ ID NO:657 and 709, for example, wherein the antibody is clazakizumab, for example, wherein the antibody is administered intravenously at a dose ranging from about 5 mg-50 mg or subcutaneously at a dose ranging from about 10 mg-50 mg.

It is another specific object of the disclosure to provide therapeutic protocols for treating acute or chronic respiratory distress syndrome and the amelioration of side effects associated therewith such as lung damage and cytokine storm in patients infected or suspected of being infected by COVID-19 by the use of specific anti-IL-6 antibodies and antibody fragments wherein the antibody or antibody fragment comprises: a variable light chain polypeptide comprising the CDRs of SEQ ID NOs:4, 5 and 6 and, and a variable heavy chain polypeptide comprising the CDRs of SEQ ID NOs:7, 8 or 120, and 9, e.g., wherein the antibody comprises a V_{H} and V_{L} polypeptide respectively at least 90, 95, 96, 97, 98 or 99% identical to the polypeptides of SEQ ID NO:657 and 709 and optionally wherein the antibody is clazakizumab, optionally wherein the antibody is administered about every week, about every 2 weeks, about every 4 weeks, 8 weeks, 12 weeks, 16 weeks, 20 weeks, or 24 weeks.

It is another specific object of the disclosure to provide therapeutic protocols for treating acute or chronic respiratory distress syndrome and the amelioration of side effects associated therewith such as lung damage and cytokine storm in patients infected or suspected of being infected by COVID-19 by the use of specific anti-IL-6 antibodies and antibody fragments wherein the antibody or antibody fragment comprises: a variable light chain polypeptide comprising the CDRs of SEQ ID NOs:4, 5 and 6 and, and a variable heavy chain polypeptide comprising the CDRs of SEQ ID NOs:7, 8 or 120, and 9, e.g., wherein the antibody comprises a V_{H} and V_{L} polypeptide respectively at least 90, 95, 96, 97, 98 or 99% identical to the polypeptides of SEQ ID NO:657 and 709, for example, wherein the antibody is clazakizumab, for example, wherein the antibody is administered immediately after detecting signs of RDS or ARDS.

It is another specific object of the disclosure to provide therapeutic protocols for treating acute or chronic respiratory distress syndrome and the amelioration of side effects associated therewith such as lung damage and cytokine storm in patients infected or suspected of being infected by COVID-19 by the use of specific anti-IL-6 antibodies and antibody fragments wherein the antibody or antibody fragment comprises: a variable light chain polypeptide comprising the CDRs of SEQ ID NOs:4, 5 and 6 and, and a variable heavy chain polypeptide comprising the CDRs of SEQ ID NOs:7, 8 or 120, and 9, e.g., wherein the antibody comprises a V_{H} and V_{L} polypeptide respectively at least 90, 95, 96, 97, 98 or 99% identical to the polypeptides of SEQ ID NO:657 and 709, for example, wherein the antibody is clazakizumab, for example, wherein the antibody is administered at a dose ranging from about 0.01 mg-5000 mg, more typically from 0.1-1000 mg, or from 1-500 mg, for example, by intravenous or subcutaneous administration.

It is another specific object of the disclosure to provide therapeutic protocols for treating acute or chronic respiratory distress syndrome and the amelioration of side effects associated therewith such as lung damage and cytokine storm in patients infected or suspected of being infected by COVID-19 by the use of specific anti-IL-6 antibodies and antibody fragments wherein the antibody or antibody fragment comprises: a variable light chain polypeptide comprising the CDRs of SEQ ID NOs:4, 5 and 6 and, and a variable heavy chain polypeptide comprising the CDRs of SEQ ID NOs:7, 8 or 120, and 9, e.g., wherein the antibody comprises a V_{H} and V_{L} polypeptide respectively at least 90, 95, 96, 97, 98 or 99% identical to the polypeptides of SEQ ID NO:657 and 709, for example, wherein the antibody is clazakizumab, and optionally wherein the antibody is administered intravenously at doses of 5 mg-50 mg or subcutaneously at doses of 10 mg-50 mg.

It is another object of the disclosure to use any of the afore-mentioned methods in combination with the standard of care immunosuppression regimens (e.g., thymoglobulin, basiliximab, mycophenolate mofetil, tacrolimus, and corticosteroids) that are normally administered to patients exhibiting signs of cytokine storm and/or RDS (i.e., either ARDS or CRDS).

It is another object of the disclosure to use any of the afore-mentioned methods, wherein the anti-IL-6 antibody or antibody fragment contains an Fc region that has been modified to alter effector function, half-life, proteolysis, and/or glycosylation.

It is another object of the disclosure to use any of the afore-mentioned methods wherein the anti-IL-6 antibody is selected from a humanized, single chain, or chimeric antibody and the antibody fragment is selected from a Fab, Fab′, F(ab′)2, Fv, or scFv.

It is another object of the disclosure to use any of the afore-mentioned methods wherein the anti-IL-6 antibody dose is between about 0.001 and 100 mg/kg of body weight of recipient patient, for example, from 0.01 to 20 mg/kg of body weight.

It is another object of the disclosure to use any of the afore-mentioned methods wherein the antibody or fragment inhibits the binding of IL-6 to gp130 and/or IL-6 binding to IL-6R1.

It is another object of the disclosure to use any of the afore-mentioned methods wherein the wherein the anti-IL-6 antibody or antibody fragment e.g., clazakizumab, comprises a human constant region.

It is another object of the disclosure to use any of the afore-mentioned methods wherein the anti-IL-6 antibody e.g., clazakizumab, comprises a human constant region such as an IgG1, IgG2, IgG3 or IgG4 constant region or comprises a human IgG1 constant region.

It is another object of the disclosure to provide a method of treating (e.g. by preventing, stabilizing or reducing) RDS (i.e., ARDS or CRDS) in a COVID-19 infected subject comprising administering to said subject a prophylactically or therapeutically effective amount of an anti-human interleukin-6 (IL-6) antibody or anti-human IL-6 antibody fragment, wherein the antibody or antibody fragment comprises: a variable light chain polypeptide comprising the CDRs of SEQ ID NOs:4, 5 and 6 and, and a variable heavy chain polypeptide comprising the CDRs of SEQ ID NOs:7, 8 or 120, and 9.

Particular, exemplary embodiments include methods of treating acute or chronic respiratory distress syndrome (ARDS or CRDS) in a human subject who has or is suspected of having a coronavirus infection, such as COVID-19, comprising administering to said subject an effective amount of an anti-human interleukin-6 (IL-6) antibody. Further embodiments include methods of reducing the risk of acute respiratory distress syndrome (ARDS) in a human subject who has or is suspected of having a coronavirus infection, such as COVID-19, comprising administering to said subject an effective amount of an anti-human interleukin-6 (IL-6) antibody. And yet further embodiments include methods of treating a human subject who has or is suspected of having a coronavirus infection, such as COVID-19, comprising administering to said subject an effective amount of an anti-human interleukin-6 (IL-6) antibody, optionally wherein the subject has mild ARDS or does not have acute respiratory distress syndrome (ARDS). As noted again below, the above treatments, in some embodiments, include reducing the severity of ARDS or CRDS, preventing its onset, reducing at least one symptom of ARDS or CRDS, and stabilizing the condition (i.e. so that it does not worsen). Thus, in some embodiments, the treated patient in the methods above does not have ARDS or CRDS at the time treatment begins, while in other embodiments, the treated patient has ARDS or CRDS at the time treatment begins, and may optionally have mild ARDS at the time treatment begins. In some cases, the treatment reduces the risk of cytokine storm syndrome, sepsis and/or organ failure in the subject.

Methods herein also include a method of treating cytokine storm syndrome in a human subject who has or is suspected of having a coronavirus infection, such as COVID-19, comprising administering to said subject an effective amount of an anti-human interleukin-6 (IL-6) antibody. In some such cases, the treatment reduces the risk of sepsis and/or organ failure in the subject. In any of the above treatment methods, the subject may have a COVID-19 infection. In some cases, the subject has pneumonia, optionally pneumonia caused by a coronavirus such as COVID-19 or caused by Streptococcus pneumoniae, Mycoplasma pneumoniae, a virus, a bacterium, or a fungus. In some cases, the subject is confirmed to be COVID-19 positive prior to treatment. In other cases, the subject is confirmed to be COVID-19 positive after starting treatment. In some cases, the subject has a COVID-19 WHO score of 7 or less, such as 4-7, 5-7, 4-6, or 4-5. In some cases, the subject has a COVID-19 WHO score of 6 or less, such as 4-6 or 4-5, and/or the subject has not been intubated. In any of the methods above, the subject may also have or is suspected to have cytokine storm syndrome.

In any of the above methods, the anti-IL-6 antibody may be administered at a dose ranging from 1-1000 mg, 1-500 mg, 5-50 mg, 10-50 mg, or at a dose of 10 mg, 12.5 mg, 15 mg, 20 mg, 25 mg, 30 mg, 35 mg, 40 mg, 45 mg, or 50 mg. In some cases, the anti-IL-6 antibody is administered at a dose ranging from 10-12.5 mg or 10-25 mg or 12.5-25 mg. In some cases, the anti-IL-6 antibody is administered at a dose of 10 mg or 12.5 mg or 25 mg. In some cases, the anti-IL-6 antibody is administered at a dose ranging from 0.01-20 mg/kg, 0.1-20 mg/kg, 0.1-1 mg/kg, or 0.1-0.5 mg/kg of body weight of the subject. In some cases, the anti-IL-6 antibody is administered to the subject only once. In other cases, the anti-IL-6 antibody is administered at least twice to the subject with at least a 48-hour interval between doses. In some cases, a 10 mg or 12.5 mg or 25 mg dose of the anti-IL-6 antibody is administered every 2 days, every 3 days, twice weekly, every 1 week, every 2 weeks, every 4 weeks or monthly. In some cases, the anti-IL-6 antibody is administered once monthly or every 4 weeks.

In any of the above treatment methods, the anti-IL-6 antibody may inhibit the binding of human IL-6 to human gp130 and/or to human IL-6R1. In any of the above methods, the antibody may comprise a light chain comprising a variable light chain polypeptide comprising light chain complementarity defining region (CDRs) comprising amino acid sequences of SEQ ID NOs: 4, 5 and 6, and a heavy chain comprising heavy chain CDRs comprising amino acid sequences of SEQ ID NOs: 7, 8 or 120, and 9. In some cases, the anti-human IL-6 antibody comprises a heavy chain comprising the amino acid sequence of SEQ ID NO: 704 or 745 and comprises a light chain comprising the amino acid sequence of SEQ ID NO: 702 or 746. In some cases, the anti-IL-6 antibody is a humanized, single chain, or chimeric antibody, or an antibody fragment (e.g., selected from a Fab, Fab′, F(ab′)2, Fv, or scFv fragment). In some cases, the anti-IL-6 antibody comprises a human constant region. In some cases, the human constant region comprises an IgG1, IgG2, IgG3 or IgG4 constant region. For example, in some cases, the anti-IL-6 antibody comprises a human IgG1 constant region. In addition, in some cases, the human constant region comprises an Fc region that has been modified to alter effector function, half-life, proteolysis, and/or glycosylation. In some cases, the anti-IL-6 antibody comprises a human IgG1 light chain constant region comprising the amino acid sequence of SEQ ID NO: 586 and a human heavy chain constant region comprising the amino acid sequence of SEQ ID NO: 588. In some cases, the anti-IL-6 antibody comprises a heavy chain comprising the amino acid sequence of SEQ ID NO: 657 and a light chain comprising the amino acid sequence of SEQ ID NO: 709. In some cases, the anti-IL-6 antibody is clazakizumab.

Additional treatment methods herein include a method of treating ARDS or CRDS in a human subject who has or is suspected of having a viral, fungal, or bacterial infection, comprising administering to said subject at least one dose of 10 mg, 12.5 mg, or 25 mg of an anti-human interleukin-6 (IL-6) antibody, optionally wherein the subject is receiving supplemental oxygen or is on a mechanical ventilator or respirator prior to treatment (e.g., for at least 24 hours prior to treatment), and optionally wherein the subject has or is suspected of having cytokine storm syndrome, wherein the anti-IL-6 antibody: (a) comprises a variable light chain polypeptide comprising light chain complementarity defining region (CDRs) comprising amino acid sequences of SEQ ID NOs: 4, 5 and 6, and a heavy chain comprising heavy chain CDRs comprising amino acid sequences of SEQ ID NOs: 7, 8 or 120, and 9; (b) comprises a heavy chain comprising the amino acid sequence of SEQ ID NO: 704 or 745 and comprises a light chain comprising the amino acid sequence of SEQ ID NO: 702 or 746; or (c) is clazakizumab. And additional treatment methods also include a method of treating cytokine storm syndrome in a human subject who has or is suspected of having a viral, fungal, or bacterial infection, comprising administering to said subject at least one dose of 10 mg, 12.5 mg, or 25 mg of an anti-human interleukin-6 (IL-6) antibody, optionally wherein the subject is receiving supplemental oxygen or is on a mechanical ventilator or respirator prior to treatment (e.g., for at least 24 hours prior to treatment), wherein the anti-IL-6 antibody: (a) comprises a variable light chain polypeptide comprising light chain complementarity defining region (CDRs) comprising amino acid sequences of SEQ ID NOs: 4, 5 and 6, and a heavy chain comprising heavy chain CDRs comprising amino acid sequences of SEQ ID NOs: 7, 8 or 120, and 9; (b) comprises a heavy chain comprising the amino acid sequence of SEQ ID NO: 704 or 745 and comprises a light chain comprising the amino acid sequence of SEQ ID NO: 702 or 746; or (c) is clazakizumab. As noted again below, these additional treatments, in some embodiments, include reducing the severity of ARDS or CRDS, preventing its onset, reducing at least one symptom of ARDS or CRDS, and stabilizing the condition (i.e., so that it does not worsen). Thus, in some embodiments, the treated patient in the methods above does not have ARDS or CRDS at the time treatment begins, while in other embodiments, the treated patient has ARDS or CRDS at the time treatment begins, and may optionally have mild ARDS at the time treatment begins. Accordingly, in some cases the subject does not have ARDS prior to treatment and the treatment reduces the risk of the subject developing ARDS. In other cases, the subject has mild ARDS prior to treatment and the treatment reduces the risk of the subject developing moderate or severe ARDS. In some cases, the subject has a coronavirus infection, e.g., a COVID-19, SARS, or MERS infection; or wherein the subject has pneumonia, optionally pneumonia caused by a coronavirus such as COVID-19 or caused by Streptococcus pneumoniae, Mycoplasma pneumoniae, a virus, a bacterium, or a fungus. Thus, in some cases, the subject has or is suspected of having a COVID-19 infection. In some cases, the subject has pneumonia. In some cases where the subject has COVID-19, the subject is confirmed to be COVID-19 positive prior to treatment. In other cases, the subject is confirmed to be COVID-19 positive after starting treatment. In some cases, the subject has a COVID-19 WHO score of 7 or less, such as 4-7, 5-7, 4-6, or 4-5. In other cases, the subject has a COVID-19 WHO score of 6 or less, such as 4-6 or 4-5, and/or wherein the subject has not been intubated. In some cases, the anti-IL-6 antibody is administered only once. In other cases, the anti-IL-6 antibody is administered at least twice to the subject with at least a 48-hour interval between doses. In some cases, a 10 mg or 12.5 mg or 25 mg dose of the anti-IL-6 antibody is administered every 2 days, every 3 days, twice weekly, every 1 week, every 2 weeks, every 4 weeks or monthly. In other cases, the anti-IL-6 antibody is administered once monthly or every 4 weeks.

In any of the above treatments herein, the anti-IL-6 antibody may be administered intravenously or subcutaneously. In any of the above treatments, in some cases the subject may show at least one of the following symptoms prior to treatment: barotrauma (volutrauma), pulmonary embolism (PE), pulmonary fibrosis, ventilator-associated pneumonia (VAP); gastrointestinal bleeding (ulcer), dysmotility, pneumoperitoneum, bacterial translocation; Hypoxic brain damage; abnormal heart rhythms, myocardial dysfunction; acute kidney failure, positive fluid balance; vascular injury, pneumothorax, tracheal injury/stenosis; malnutrition (catabolic state), electrolyte abnormalities; Atelectasis, blood clots, weakness in muscles used for breathing, stress ulcers, depression or other mental illness; single or multiple organ failure; pulmonary hypertension or increase in blood pressure in the main artery from the heart to the lungs.

In any of the above treatments, in some cases, the subject is receiving supplemental oxygen prior to treatment (e.g., for at least 24 hours prior to treatment), such as via a respirator or a ventilator (e.g., a non-invasive or an invasive ventilator). In some cases, subject is not intubated and is not on an invasive ventilator prior to treatment.

In any of the above treatments, in some cases, levels of IL-6 in the subject are detected prior to treatment. In some instances, levels of IL-6 in the patient are detected and confirmed to be elevated prior to treatment, e.g., are greater than 20 pg/mL, greater than 25 pg/mL, greater than 30 pg/mL, or greater than 35 pg/mL prior to treatment. In some cases, levels of C-reactive protein (CRP) in the subject are detected prior to treatment. In some cases, levels of CRP in the patient are detected and confirmed to be elevated prior to treatment, e.g., a CRP level greater than 35 mg/L, greater than 50 mg/L, or greater than 100 mg/L. In some cases, IL-6 and/or CRP levels in the subject are detected during and/or after treatment, optionally in addition to detection of the levels prior to treatment. For example, levels before and after treatment may be compared in some cases to determine if a further dose of antibody should be administered. In some instances, for example, a first 25 mg dose of clazakizumab is administered to the subject, and a second dose is administered 24-48 h later if the C-reactive protein (CRP) level in the subject fails to decrease by at least 20%, at least 30%, at least 40%, at least 50%, at least 60%, at least 70%, at least 80%, or at least 90%, 24-48 h after the first dose. In some instances, a first 25 mg dose of clazakizumab is administered to the subject, and a second dose is administered 24-48 h later if the C-reactive protein (CRP) level in the subject fails to decrease by at least 50% 24-48 h after the first dose. In some cases, a first 25 mg dose of clazakizumab is administered to the subject, and a second dose is administered if the C-reactive protein (CRP) level in the subject fails to decrease by at least 50% 48 h after the first dose.

In any of the above treatments herein, the subject may have at least one or at least two of the following prior to treatment: CRP>35 mg/L; ferritin>500 ng/mL; D-dimer>1 pg/L; neutrophil-lymphocyte ratio>4; LDH>200 U/L; and increased troponin level without known cardiac disease.

In any of the above methods, the subject may also be administered at least one additional therapeutic. In some cases, the subject receives one or more of corticosteroids; inhaled nitric oxide (NO); extracorporeal membrane oxygenation (venovenous or venoarterial), or an immunosuppressive agent, optionally thymoglobulin, basiliximab, mycophenolate mofetil, tacrolimus, an anti-CD20 antibody such as rituximab. In some cases, the subject is further treated with a Pneumocystis jiroveci pneumonia (PJP) therapeutic, e.g., trimethoprim (e.g., 80 mg daily pill), and/or sulfamethoxazole (e.g., 160 mg 3 times weekly pill), inhaled pentamidine or oral dapsone (optionally commenced within at least 1 week of treatment). In some cases, the subject is treated with a pulse steroid such as oral prednisone (e.g., at 200 mg/day). In some cases, the subject is further treated with one or more standard of care immunosuppression regimens (e.g., thymoglobulin, basiliximab, mycophenolate mofetil, tacrolimus, corticosteroids). In some cases, the subject is additionally treated with an antiviral, an antibiotic, or an immunosuppressive agent. In some cases, the subject optionally is further treated with any of the following: (a) azathioprine (e.g., 1.0-2.0 mg/kg/day), (b) calcineurin inhibitors (CNIs), (c) mycophenolate mofetil (MMF) (e.g., 1.0-2.0 g/day)/mycophenolic acid (MPA) (e.g., 720-1440 mg/day), (d) mTOR inhibitors (e.g., tacrolimus (e.g., target trough levels 5-8 ng/ml), everolimus, sirolimus), (e) low dose corticosteroids (e.g., prednisone/prednisolone≤10 mg/day), (f) antihypertensive agents (e.g., angiotensin converting enzyme inhibitors (ACEIs), (g) angiotensin II receptor blockers (ARBs), (h) cyclosporine, (e.g., target trough levels 50-150 ng/ml), (i) antidiabetogenic agents, or (j) or a combination of any of the previous.

In any of the methods above, the subject may also have one or more of the following characteristics: (a) over 60, 65, 70 years of age, (b) type 1 or type 2 diabetes, (c) high blood pressure, (d) cancer, (e) an inflammatory lung condition, e.g., asthma, COPD or cystic fibrosis, (f) arteriosclerosis, and/or (g) an inflammatory or autoimmune condition.

In any of the methods herein, the anti-IL-6 antibody is administered intravenously or subcutaneously. In some cases, it is administered intravenously. In others, it is administered subcutaneously.

In any of the methods herein, the subject may have improved or normal lung function after treatment, such as 4 weeks (28 days) or 1 month after treatment, or 8 weeks (60 days) or 2 months after treatment. In some cases, the treatment eliminates the need for the subject to go on a ventilator or reduces the time the subject is on a ventilator (e.g., an invasive ventilator). In some cases, the treatment may reduce the time that a subject needs to be on supplemental oxygen. In some cases, methods herein result in one or more of the following compared to placebo: (i) time to liberation from mechanical ventilation is reduced; (ii) time to durable fever resolution is reduced, (iii) time to durable improvement of oxygenation is reduced, (iv)C-reactive protein (CRP) response is reduced, (v) length of ICU stay is reduced, or (vi) number of subjects who survive 28 days post treatment is increased. In some methods herein, the subject is hospitalized but does not exhibit pulmonary or respiratory difficulties requiring exogenous high levels of oxygen. In some cases, method herein may (i) reduce the number of COVID-19 infected subjects who develop ARDS; (ii) slow the onset of ARDS in COVID-19 infected subjects and/or (iii) result in an ARDS condition in COVID-19 infected subjects which is less severe than in subjects not administered the anti-IL-6 antibody (i.e. mild or moderate ARDS vs. severe ARDS or mild ARDS vs. moderate or severe ARDS). In some method herein, the subject: (i) is intubated and mechanically ventilated with acute respiratory distress syndrome (ARDS), and requires vasopressor support; (ii) has profound hypoxemia requiring supported by non-invasive ventilationory (NIV) support; (iii) is a solid organ recipient, optionally a kidney or heart recipient; (iv) shows sign of renal failure, optionally acute renal failure; (v) has elevated IL-6; (vi) has increased higher D-dimer levels, (vii) has increased fibrinogen levels and/or (viii) has increased ferritin levels; or a combination of any of the foregoing; wherein said increases, if present, are relative to median levels observed in normal, non-inflammatory persons.

### DETAILED DESCRIPTION

There is a need in the art for novel and improved methods the treatment of acute or chronic respiratory distress syndrome and the amelioration of side effects associated therewith such as lung damage, cytokine storm and organ failure which often results in death in patients infected by COVID-19 or another viral, fungal, or bacterial or other pathogen which causes ARDS or CRDS, e.g., in patients already exhibiting signs of lung damage or other symptoms associated with ARDS or CRDS who optionally may comprise other risk factors for ARDS or CRDS such as pneumonia, asthma or another inflammatory lung condition, diabetes, etc., as there is a shortage of effective treatments for such conditions. This is severely needed as such conditions may result in permanent lung damage and in extreme circumstances the patient needs to be put on a respirator or ventilator and if uncontrolled this condition often results in death.

Particularly the present disclosure addresses these needs by the use of specific anti-IL-6 antibodies and antibody fragments wherein the antibody or antibody fragment comprises: a variable light chain polypeptide comprising the CDRs of SEQ ID NOs:4, 5 and 6 and, and a variable heavy chain polypeptide comprising the CDRs of SEQ ID NOs:7, 8 or 120, and 9, e.g., wherein the antibody comprises a V_{H} and V_{L} polypeptide respectively at least 90, 95, 96, 97, 98 or 99% identical to the polypeptides of SEQ ID NO:657 and 709, for example, wherein the antibody is clazakizumab, that effectively treat (e.g., inhibit or reverse) ARDS or CRDS and symptoms associated therewith, e.g., cytokine storm and lung damage.

Interleukin-6 (IL-6) is a cytokine with powerful stimulatory effects on B cells and plasma cells and is responsible, in conjunction with other cytokines, for normal antibody production. IL-6 also has powerful stimulatory effects on T-cell mediated inflammatory processes.

This disclosure relates to the use of specific anti-IL-6 antibodies or antibody fragments to treat acute or chronic respiratory distress syndrome and ameliorate side effects associated therewith such as lung damage and cytokine storm in patients infected or suspected of being infected by COVID-19 or another viral or bacterial or other pathogen. In particular the disclosure pertains to methods of improving lung function and/or reversing or preventing lung damage and/or cytokine storm and improving survival and quality of life in COVID-19 infected patients who have or exhibit signs of ARDS or CRDS or are at risk for developing COVID-19 associated ARDS or CRDS, e.g., because of other risk factors such as advanced age (over 60 or 70 years of age), other conditions such as other lung conditions such pneumonia, asthma, COPD, cystic fibrosis, cancer, diabetes, high blood pressure or other inflammatory or autoimmune conditions, especially those which adversely affect lung function.

Particularly the present disclosure addresses these needs by the use of specific anti-IL-6 antibodies and antibody fragments, for example, wherein the antibody or antibody fragment comprises: a variable light chain polypeptide comprising the CDRs of SEQ ID NOs:4, 5 and 6 and, and a variable heavy chain polypeptide comprising the CDRs of SEQ ID NOs:7, 8 or 120, and 9, e.g., wherein the antibody comprises a V_{H} and V_{L} polypeptide respectively at least 90, 95, 96, 97, 98 or 99% identical to the polypeptides of SEQ ID NO:657 and 709, for example, wherein the antibody is clazakizumab, that effectively treat (e.g., inhibit or reverse) ARDS or CRDS and symptoms associated therewith, e.g., cytokine storm and ARDS or CRDS associated lung damage.

In some instances, the treated patients who are treated with specific anti-IL-6 antibodies and antibody fragments according to the disclosure are individuals suspected of having COVID-19 infection, i.e., a definitive test result is not yet available, but they are suspected because of contact with other individuals and/or symptoms associated with COVID-19 infection such as fever, dry cough, breathing difficulties, et al.

In some instances, the COVID-19 or other infected patients who are treated with specific anti-IL-6 antibodies and antibody fragments according to the disclosure are individuals who are already exhibiting signs of cytokine storm and/or lung damage associated with ARDS or CRDS.

In some instances the COVID-19 or other infected patients who are treated with specific anti-IL-6 antibodies and antibody fragments are individuals who have or are individuals suspected of having COVID-19 infection, who have another respiratory or lung condition such as pneumonia caused by COVID-19 or another bacterial or viral pathogen which further disposes the patient to developing ARDS or CRDS, e.g., so severe it may require use of a ventilator or respirator or another form of supplemental oxygen.

In some instances, the COVID-19 or other infected patients who are treated with specific anti-IL-6 antibodies and antibody fragments are individuals who have or are individuals suspected of having COVID-19 infection, who have another respiratory or lung condition, including those who have lung problems so severe that they are on a ventilator or respirator.

In some instances the COVID-19 or other infected patients who are treated with specific anti-IL-6 antibodies and antibody fragments are individuals who have or are individuals suspected of having COVID-19 infection, who are being treated with other therapeutics or regimens used to treat ARDS or CRDS or infection such as steroids, other immunosuppressives, e.g., thymoglobulin, basiliximab, mycophenolate mofetil, tacrolimus, an anti-CD20 mAb such as rituximab, corticosteroids, antivirals, antibiotics, et al.

In particular, the disclosure provides novel therapeutic protocols for treating ARDS or CRDS and symptoms associated therewith in COVID-19 infected patients in need thereof by the use of an anti-IL-6 antibody such as clazakizumab.

Also the disclosure relates to the use of specific anti-IL-6 antibodies or antibody fragments to treat acute or chronic respiratory distress syndrome and ameliorate side effects associated therewith such as lung damage and cytokine storm in patients infected or suspected of being infected by COVID-19 or another viral or bacterial or other pathogen, comprising administering a prophylactically or therapeutically effective amount of an anti-human interleukin-6 (IL-6) antibody or antibody fragment, wherein the antibody or antibody fragment comprises: a variable light chain polypeptide comprising the CDRs of SEQ ID NOs:4, 5 and 6 and, and a variable heavy chain polypeptide comprising the CDRs of SEQ ID NOs:7, 8 or 120, and 9, e.g., wherein the antibody comprises a VH and VL polypeptide respectively at least 90, 95, 96, 97, 98 or 99% identical to the polypeptides of SEQ ID NO:657 and 709, or the antibody comprises a heavy chain and light chain polypeptide respectively at least 90, 95, 96, 97, 98 or 99% identical to the polypeptides of SEQ ID NO:704 and 702, in some cases wherein the antibody is clazakizumab.

In some embodiments the anti-IL-6 antibodies contain specific CDRs, as described in U.S. Pat. No. 9,452,227, the disclosure of which is hereby incorporated by reference in its entirety. In some embodiments, an anti-IL-6 antibody is a humanized variant of Ab1 described in that disclosure (see, e.g., column 46, line 8, to column 47, line, 12, of U.S. Pat. No. 9,452,227), e.g., clazakizumab, or an antibody or antibody fragment that specifically binds to the same linear or conformational epitope(s) on an intact human IL-6 polypeptide fragment thereof as clazakizumab or one comprising the same CDRs as this antibody.

Exemplary anti-IL-6 antibodies and antibody fragments comprise: a variable light chain polypeptide comprising the CDRs of SEQ ID NOs: 4, 5 and 6 and possessing at least 90% identity to the variable light chain polypeptide of SEQ ID NO: 709, and a variable heavy chain polypeptide comprising the CDRs of SEQ ID NOs: 7, 8 or 120, and 9 and possessing at least 90% identity to the variable heavy chain polypeptide of SEQ ID NO: 657, wherein the antibody or antibody fragment specifically binds to IL-6 and antagonizes one or more activities associated with IL-6 and specifically binds to the same epitope(s) on IL-6 as an anti-IL-6 antibody comprising the variable light chain polypeptide in of SEQ ID NO: 709 and the variable heavy chain polypeptide of SEQ ID NO: 657. (All of the sequences identified herein are described in U.S. Pat. No. 9,452,227).

In some embodiments the anti-IL-6 antibody used in the inventive methods is clazakizumab. Clazakizumab is a humanized monoclonal antibody that binds to and inhibits human IL-6. This antibody potently inhibits or prevents IL-6 from binding to IL-6R and to gp130. Clazakizumab has demonstrated efficacy in clinical and pre-clinical trials evaluating patients with rheumatoid arthritis, psoriatic arthritis, cancer and cachexia among other conditions.

Treatment with the subject anti-IL-6 antibodies e.g., clazakizumab, may prevent, inhibit or treat ARDS and CRDS and associated symptoms such as breathing difficulties, reduced lung function, increased IL-6 and/or CRP levels, cytokine storm, etc.

### Definitions

In this application, the use of “or” means “and/or” unless stated otherwise. In the context of a multiple dependent claim, the use of “or” refers back to more than one preceding independent or dependent claim in the alternative only.

As described herein, any concentration range, percentage range, ratio range or integer range is to be understood to include the value of any integer within the recited range and, when appropriate, fractions thereof (such as one tenth and one hundredth of an integer), unless otherwise indicated.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Instead, the section headings used herein are for organizational purposes only.

The term “antibody” as used herein (abbreviated “Ab”) refers to a molecule comprising at least complementarity-determining region CDR1, CDR2, and CDR3 of a heavy chain and at least CDR1, CDR2, and CDR3 of a light chain, wherein the molecule is capable of binding to antigen, i.e., to human IL-6. The term antibody includes various antibody fragments that are capable of binding antigen, such as Fv, single-chain Fv (scFv), Fab, Fab′, and (Fab′)2. Thus, an “anti-IL-6 antibody” encompasses molecules falling within the scope of the term “anti-IL-6 antibody or anti-IL-6 antibody fragment.” The term “antibody” also encompasses molecules with full length heavy and/or light chains. The term antibody also includes chimeric antibodies, humanized antibodies, bispecific antibodies, antibody-drug conjugates, and the like.

In some embodiments, an antibody comprises a heavy chain variable region and a light chain variable region. In some embodiments, an antibody comprises at least one heavy chain comprising a heavy chain variable region and at least a portion of a heavy chain constant region, and at least one light chain comprising a light chain variable region and at least a portion of a light chain constant region. In some embodiments, an antibody comprises two heavy chains, wherein each heavy chain comprises a heavy chain variable region and at least a portion of a heavy chain constant region, and two light chains, wherein each light chain comprises a light chain variable region and at least a portion of a light chain constant region. As used herein, a single-chain Fv (scFv), or any other antibody that comprises, for example, a single polypeptide chain comprising all six CDRs (three heavy chain CDRs and three light chain CDRs) is considered to have a heavy chain and a light chain. In some such embodiments, the heavy chain is the region of the antibody that comprises the three heavy chain CDRs and the light chain in the region of the antibody that comprises the three light chain CDRs.

The term “interleukin 6” or “IL-6” herein refers to the human IL-6 protein unless expressly stated otherwise (e.g., murine IL-6). Accordingly, an “anti-IL-6 antibody” herein and an “anti-human IL-6 antibody” herein are considered equivalent terms unless expressly clarified otherwise, as covering antibodies that bind to the human IL-6 protein.

The term “isolated” as used herein refers to a molecule, such as an antibody, that has been separated from at least some of the components with which it is typically found in nature. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide.

The words “subject” and “patient” herein are used interchangeably and refer to a human.

As used herein, “Acute respiratory distress syndrome” or “ARDS” refers to a type of respiratory failure characterized by rapid onset of widespread inflammation in the lungs. Symptoms include shortness of breath, rapid breathing, and bluish skin coloration. Among those who survive, a decreased quality of life is relatively common. Known causes may include sepsis, pancreatitis, trauma, pneumonia, and aspiration. The underlying mechanism involves diffuse injury to cells, which form the barrier of the microscopic air sacs of the lungs, surfactant dysfunction, activation of the immune system, and dysfunction of the body's regulation of blood clotting. In effect, ARDS impairs the lungs' ability to exchange oxygen and carbon dioxide. Diagnosis may be based on a PaO₂/FiO₂ ratio of less than 300 mmHg despite a PEEP of more than 5 cm H2O; and heart related pulmonary edema, as the cause, must be excluded. The primary treatment involves mechanical ventilation together with treatments directed at the underlying cause. Ventilation strategies include using low volumes and low pressures. If oxygenation remains insufficient, lung recruitment maneuvers and neuromuscular blockers may be used. If this is insufficient, ECMO may be an option. The syndrome is associated with a death rate between 35 and 50%. Globally, ARDS affects more than 3 million people a year. The condition was first described in 1967. Although the terminology of “adult respiratory distress syndrome” has at times been used to differentiate ARDS from “infant respiratory distress syndrome” in newborns, the international consensus is that “acute respiratory distress syndrome” is the best term because ARDS can affect people of all ages. There are modified diagnostic criteria for children and areas of the world with fewer resources.

This definition of “ARDS” applies for all embodiments throughout this application, but in individual studies described in the experimental section, some diagnosis criteria may differ in part from the above when describing the particular study, e.g. for exclusion purposes diagnosis of ARDS may be based a lower PaO₂/FiO₂ ratio than shown above, for example, to exclude patients with mild ARDS or to include patients only with severe ARDS. ARDS also includes mild, moderate, and severe forms. Specifically, ARDS may be classified into mild, moderate and severe ARDS according to the “Berlin Definition” (Ranieri V M, et al. Acute respiratory distress syndrome: the Berlin definition. JAMA. 2012; 307(23):2526-33). Mild ARDS as defined herein encompasses subjects with a PaO₂/FiO₂ ratio of from 200 to <300 mmHg. Moderate ARDS as defined herein encompasses subjects with a PaO₂/FiO₂ ratio of 100 to <200 mmHg. Severe ARDS encompasses subjects with a PaO₂/FiO₂ ratio of <100 mmHg. Moderate and severe ARDS subjects herein, therefore encompass those with a PaO₂/FiO₂ ratio of <200 mmHg.

Complications of ARDS may include the following:
Lungs: barotrauma (volutrauma), pulmonary embolism (PE), pulmonary fibrosis, ventilator-associated pneumonia (VAP)
Gastrointestinal: bleeding (ulcer), dysmotility, pneumoperitoneum, bacterial translocation
Neurological: Hypoxic brain damage
Cardiac: abnormal heart rhythms, myocardial dysfunction
Kidney: acute kidney failure, positive fluid balance
Mechanical: vascular injury, pneumothorax (by placing pulmonary artery catheter), tracheal injury/stenosis (result of intubation and/or irritation by endotracheal tube)
Nutritional: malnutrition (catabolic state), electrolyte abnormalities.

Other complications that are typically associated with ARDS include:
Atelectasis: small air pockets within the lung collapse
Complications that arise from treatment in a hospital: blood clots formed by lying down for long periods of time, weakness in muscles that are used for breathing, stress ulcers, and even depression or other mental illnesses.
Failure of multiple organs
Pulmonary hypertension or increase in blood pressure in the main artery from the heart to the lungs. This complication typically occurs due to the restriction of the blood vessel due to inflammation of the mechanical ventilation.

Current treatments for ARDS include treatment with corticosteroids; inhaled nitric oxide (NO); extracorporeal membrane oxygenation which comprises mechanically applied prolonged cardiopulmonary support. There are two types of ECMO: Venovenous which provides respiratory support and venoarterial which provides respiratory and hemodynamic support. People with ARDS who do not require cardiac support typically undergo venovenous ECMO.

As used herein, “chronic respiratory distress syndrome” or “Chronic respiratory disease” or “CRDS” refers to long-term diseases of the airways and other structures of the lung. They are characterized by a high inflammatory cell recruitment (neutrophil) and/or destructive cycle of infection, (e.g. mediated by Pseudomonas aeruginosa). Some of the most common are asthma, chronic obstructive pulmonary disease, and acute respiratory distress syndrome. CRDS is not curable; however, various forms of treatment that help dilate major air passages and improve shortness of breath can help control symptoms and increase the quality of life.

The term “respiratory distress syndrome” or “RDS” herein encompasses both ARDS and CRDS.

As used herein “pneumonia” refers to an inflammatory condition of the lung affecting primarily the small air sacs known as alveoli. Typically, symptoms include some combination of productive or dry cough, chest pain, fever and difficulty breathing. The severity of the condition is variable. Pneumonia is usually caused by infection with viruses or bacteria and less commonly by other microorganisms and fungi, certain medications, or conditions such as autoimmune diseases. Coronaviruses and specifically COVID-19 may also cause pneumonia. Risk factors for pneumonia include cystic fibrosis, chronic obstructive pulmonary disease (COPD), asthma, diabetes, heart failure, a history of smoking, a poor ability to cough such as following a stroke and a weak immune system.

Diagnosis of pneumonia is often based on the symptoms and physical examination. Chest X-ray, blood tests, and culture of the sputum may help confirm the diagnosis. The disease may be classified by where it was acquired, such as community- or hospital-acquired or health care-associated pneumonia. The most common cause is bacteria, particularly Streptococcus pneumoniae. Worldwide, tuberculosis is an important cause of pneumonia. Other pathogens such as viruses and fungi can cause pneumonia for example severe acute respiratory syndrome and pneumocystis pneumonia. A pneumonia may develop complications such as a lung abscess, a round cavity in the lung caused by the infection, or may spread to the pleural cavity.

The term “cytokine storm” or “cytokine storm syndrome” (also called “hypercytokinemia”) refers to a symptom of certain infections, in which the body's own immune system becomes over-active, for example, due to an uncontrolled release of cytokines or other pro-inflammatory molecules. Because pro-inflammatory factors may promote cell death, a cytokine storm, for example, can cause excess death of normal, bodily cells, potentially leading to serious and life-threatening consequences such as sepsis and/or multiple-organ failure. A cytokine storm can, in some cases, occur together with reduced lung function such as ARDS or CRDS after infection with certain types of infectious agents such as influenza viruses, coronaviruses such as COVID-19 (SARS-COV-2), SARS, MERS, pneumonia-causing agents, and the like. In some embodiments, a cytokine storm may be characterized at least in part by elevated levels of IL-6, or elevated levels of IL-6 and C-reactive protein (CRP).

In some embodiments, a subject herein has a “coronavirus” infection. A “coronavirus” herein refers to an RNA virus of the coronavirus classification. Examples include certain viruses causing disease outbreaks such as SARS (severe acute respiratory syndrome; SARS-CoV), MERS (Middle East respiratory syndrome; MERS-CoV), and SARS-CoV-2 (also called COVID-19). In some embodiments, a subject is diagnosed (i.e., confirmed positive) with a particular infection prior to treatment (e.g., by a PCR test or ELISA test or other type of test that detects the presence of viral DNA or protein in the body). In some embodiments, a subject is diagnosed with at least one symptom of infection with an infectious agent prior to treatment, such as, for instance, cytokine storm, reduced lung function, difficulty breathing, fever, loss of taste or smell, persistent cough, low blood-oxygen level, and the like.

As used herein, “treat,” “treating,” and “treatment” refer to therapeutic treatment, for example, wherein the object is to reduce (lessen), stabilize, or prevent onset of the targeted pathologic condition or disorder, such as ARDS, CRDS, cytokine storm syndrome, etc., as well as, for example, wherein the object is to inhibit recurrence of the condition or disorder. In certain embodiments, the term “treatment” covers administration of a therapeutic to treat a condition such as ARDS or CRDS or cytokine storm syndrome, for example. In some embodiments, the terms “treat,” “treating,” and “treatment” cover administration of a therapeutic to prevent, stabilize, or reduce a condition such as ARDS or CRDS or cytokine storm syndrome. In some embodiments, the terms cover improvement of at least one symptom of the condition in a subject. Symptoms of ARDS and CRDS, for example, may include breathing difficulties, reduced lung function, need for supplemental oxygen, elevated IL-6 and/or CRP levels, cytokine storm, etc. In some embodiments the term “treatment” relates to administration of a therapeutic to reduce (lessen) or stabilize ARDS or CRDS progression after onset of ARDS or CRDS.

“Administering” refers to the physical introduction of a composition comprising a therapeutic agent to a subject, using any of the various methods and delivery systems known to those skilled in the art.

A patient who “is suspected of having” a particular condition is an individual for whom a definitive test result or other definitive diagnostic confirmation of having the condition is not available, but, because of symptoms associated with the condition or because of contact with one or more other individuals having the condition (or a mixture of both depending on circumstances), the patient is believed likely to have the condition. For example, some patients may present symptoms indicating the condition although the condition, such as a particular infection, may not have been confirmed (e.g., through a PCR or ELISA test) prior to treatment. For instance, in some cases a test result may take several days but the patient requires treatment intervention immediately. In some cases, a definitive diagnosis may be deemed unnecessary if the patient's symptoms themselves are sufficient to strongly indicate that the patient has the condition. Symptoms associated with COVID-19 infection, for example, may include fever, dry cough, breathing difficulties, loss of taste, loss of sense of smell, and low blood oxygen level (e.g., an SpO2 of less than 90% as measured by a pulse oximeter), among others. Even if symptoms are not specific for COVID-19, one or more of these symptoms may be sufficient for suspected COVID-19, particularly if the patient has also had known contact with one or more persons with COVID-19 or if incidence of COVID-19 is high in an area of residence.

The term “elevated level” or “increased level” means a higher level of a protein or other marker in a subject relative to the same tissue in a control, such as an individual or individuals who are not suffering from the disease or other condition described herein. The elevated level may be the result of any mechanism, such as increased expression, increased stability, decreased degradation, increased secretion, decreased clearance, etc., of the protein or marker.

The term “effective amount” or “therapeutically effective amount” refers to an amount of a drug effective to treat a disease or disorder in a subject. In certain embodiments, an effective amount refers to an amount effective, at dosages and for periods of time necessary, to achieve the desired therapeutic or prophylactic result or improvement in a particular symptom or characteristic.

In some embodiments, a subject is receiving “supplemental oxygen.” This term is intended to be interpreted in the broadest sense, covering any means of supplying additional oxygen to the patient, such as through use of an oxygen mask or tank, a high-oxygen concentration room or environment, a respirator, or a ventilator, which may be noninvasive (without requiring intubation) or invasive (requiring intubation), and other means. In some embodiments, supplemental oxygen is through a non-invasive means, i.e., a means that does not require intubation.

As used herein, “improved,” “improvement,” and other grammatical variants, includes any beneficial change resulting from a treatment. A beneficial change is any way in which a patient's condition is better than it would have been in the absence of the treatment. “Improved” includes prevention of an undesired condition, slowing the rate at which a condition worsens, delaying the development of an undesired condition, and increasing the rate at which a desired condition is reached. For example, improvement in an ARDS patient encompasses any decrease in inflammatory cytokines (e.g., IL-6 or C-reactive protein (CRP)) as any increase in the amount or rate at which inflammatory cytokines are prevented, removed or reduced. For another example, improvement in a ARDS patient or patient at risk of ARDS encompasses any prevention, decrease, delay or slowing in the rate of the condition and cytokine mediated damage or loss of function, e.g., to lung function. Another exemplary improvement for a COVID-19 subject receiving treatment, for example, is a reduction of two or more in a WHO score (e.g., from 7 to 5, and the like).

### Particular Exemplary Treatment Methods

Anti-IL-6 antibodies e.g., clazakizumab, can be administered to a patient diagnosed or suspected of having COVID-19 infection who may comprise elevated IL-6 and/or CRP levels and/or other risk factors associated with ARDS or CDRS or a poor prognosis such as advanced age, cancer, another lung condition such as COPD, cystic fibrosis, another lung fibrotic condition or asthma, an autoimmune or inflammatory condition such as diabetes, pneumonia, high blood pressure, arteriosclerosis, among others. In some embodiments the anti-IL-6 antibodies e.g., clazakizumab, are be administered intravenously (e.g., at doses ranging from 0.01-5000 mg, more typically from 0.1-1000 mg or 1-500 mg, and in exemplary embodiments from 5 mg-50 mg) or via subcutaneous injection (e.g., at doses ranging from 0.01-5000 mg, more typically from 0.1-1000 mg or 1-500 mg, and in exemplary embodiments at doses of 10 mg-50 mg) every 4 weeks, starting at detection of ARDS or CRDS or detection of the risk of developing ARDS or CRDS in a patient having or suspected of having COVID-19 infection.

ARDS patients who are treated with anti-IL-6 antibodies e.g., clazakizumab, can be administered with or without one or more additional immunosuppressive agents, and the antibodies can be administered intravenously ((e.g., at doses ranging from 0.01-5000 mg, more typically from 0.1-1000 mg or 1-500 mg, and in exemplary embodiments at doses of 5 mg-50 mg) or via subcutaneous injection ((e.g., at doses ranging from 0.01-5000 mg, more typically from 0.1-1000 mg or 1-500 mg, and in exemplary embodiments at doses of 10 mg-50 mg) every 4 weeks, starting at detection of ARDS or CRDS or detection of the risk of developing ARDS or CRDS in a patient having or suspected of having COVID-19 infection. Treatment with anti-IL-6 antibodies may be continued as long as COVID-19 infection persists and/or as long as the patient exhibits signs of ARDS and/or is at risk of developing ARDS because of other risk factors such as afore-mentioned.

In some embodiments, a patient with ARDS or CRDS is treated, in particular after onset of ARDS or CRDS. In some embodiments, a patient with cytokine storm syndrome is treated. In some embodiments, a patient with ARDS or CRDS as well as cytokine storm syndrome is treated. In some embodiments, the patient is receiving supplemental oxygen, or is on a mechanical ventilator or respirator prior to treatment, such as at least for 6, 12, 18 or 24 hours prior to treatment. In some embodiments, the patient has or is suspected of having a viral, fungal, or bacterial, or parasitic infection, such as, in some embodiments, a coronavirus infection or other infection known to cause ARDS or CRDS or cytokine storm syndrome. In some embodiments, the patient has or is suspected of having pneumonia, influenza, SARS, MERS, COVID-19, or another coronavirus infection. In some embodiments, the patient has COVID-19. In some embodiments, the patient has elevated levels of IL-6, such as greater than 20 pg/mL, greater than 25 pg/mL, greater than 30 pg/mL, or greater than 35 pg/mL, prior to treatment. In some embodiments, the patient has elevated C-reactive protein (CRP) prior to treatment, such as greater than 35 mg/L, greater than 50 mg/L, or greater than 100 mg/L. In some embodiments, a subject whose IL-6 and/or CRP are elevated is given one dose of anti-IL-6 antibody such as clazakizumab, and then levels of IL-6 and/or CRP are re-checked after 24-48 hours and if not decreased by at least 20%, at least 30%, at least 40%, at least 50%, at least 60%, at least 70%, at least 80%, at least 90%, then a second dose of antibody is administered, for example within 24 or 48 hours after the first dose. In some embodiments, a subject whose IL-6 and/or CRP are elevated is given one dose of anti-IL-6 antibody such as clazakizumab, and then levels of IL-6 and/or CRP are re-checked after 24-48 hours and if not decreased by at least 50%, then a second dose of antibody is administered, for example within 24 or 48 hours after the first dose. In some embodiments, only one dose of antibody is administered. In other embodiments, a second dose is administered. In yet further embodiments, more than two doses are administered.

In some of the above embodiments, an IL-6 antibody is administered to a subject having ARDS or CRDS or cytokine storm syndrome, or a combination of ARDS or CRDS and a cytokine storm syndrome, optionally wherein the subject is receiving supplemental oxygen or is on a mechanical ventilator or respirator prior to treatment (e.g., for at least 24 hours prior to treatment), wherein the anti-IL-6 antibody (a) comprises a variable light chain polypeptide comprising light chain complementarity defining region (CDRs) comprising amino acid sequences of SEQ ID NOs: 4, 5 and 6, and a heavy chain comprising heavy chain CDRs comprising amino acid sequences of SEQ ID NOs: 7, 8 or 120, and 9; (b) comprises a heavy chain comprising the amino acid sequence of SEQ ID NO: 704 or 745 and comprises a light chain comprising the amino acid sequence of SEQ ID NO: 702 or 746; (c) comprises a heavy chain comprising the amino acid sequence of SEQ ID NO: 657 and comprises a light chain comprising the amino acid sequence of SEQ ID NO: 709; or (d) is clazakizumab, and wherein the antibody is administered at least once at a dose of 10-25 mg, 10 mg, 12.5 mg, or 25 mg, or an equivalent dose by weight. The antibody may be administered more than once, for example, if IL-6 levels and/or CRP levels do not decrease by at least 20%, at least 30%, at least 40%, at least 50%, at least 60%, at least 70%, at least 80%, at least 90%, within 24 or 48 hours after the first dose. In such cases, the antibody may be administered again 48 hours after the first dose. In some such embodiments, the subject has a coronavirus infection, e.g., a COVID-19, SARS, or MERS infection; or the subject has pneumonia, optionally pneumonia caused by a coronavirus such as COVID-19 or caused by Streptococcus pneumoniae, Mycoplasma pneumoniae, a virus, a bacterium, or a fungus. In some cases, the subject is also given at least one other therapeutic agent or regimen in addition to the anti-IL-6 antibody, such as an antiviral, antibacterial, or immunosuppressive agent, e.g., one or more standard of care immunosuppression regimens (e.g., thymoglobulin, basiliximab, mycophenolate mofetil, tacrolimus, corticosteroids), or generally one or more of corticosteroids; inhaled nitric oxide (NO); extracorporeal membrane oxygenation (venovenous or venoarterial), thymoglobulin, basiliximab, mycophenolate mofetil, tacrolimus, or an anti-CD20 antibody such as rituximab. In some embodiments, the subject has improved or normal lung function after treatment, such as 28 days or 1 month after the start of treatment. In some embodiments, the subject may have one or more of the following: an SpO₂ of <90%, supplemental oxygen treatment for at least 24 hours, an IL-6 level of at least 20 pg/mL, a CRP level of >35 mg/L, a ferritin level of >500 ng/mL, a D-dimer of >1 mg/L, a neutrophil-lymphocyte ration of >4, an LDH level of >200 U/L, and/or an increase in troponin without known cardiac disease. In some embodiments, the patient may have an infection as well as be over 60, 65, or 70 years of age, have type 1 or 2 diabetes, have high blood pressure, have cancer, have an inflammatory lung condition (e.g., asthma, COPD, cystic fibrosis), have arteriosclerosis, have an inflammatory condition, or have an autoimmune condition.

In some embodiments, a subject with ARDS and/or cytokine storm and that has or is suspected of having an infection is treated with clazakizumab at a dose of 10-25 mg, 10 mg, 12.5 mg, or 25 mg, or an equivalent dose by weight, once and, if ARDS or cytokine storm symptoms do not appear to reduce, or if IL-6 and/or CRP levels do not reduce by at least 50% within 48 hours, the subject is given at least one additional dose. In some embodiments, a subject with ARDS and/or cytokine storm and that has or is suspected of having an infection is treated with clazakizumab at a dose 25 mg, or an equivalent flat dose, once and, if ARDS or cytokine storm symptoms do not appear to reduce, or if IL-6 and/or CRP levels do not reduce by at least 50% within 48 hours, the subject is given at least one additional dose. In some embodiments, a subject with ARDS and/or cytokine storm and that has or is suspected of having a coronavirus infection (e.g., COVID-19, SARS, MERS) is treated with clazakizumab at a dose 25 mg, or an equivalent flat dose, once and, if ARDS or cytokine storm symptoms do not appear to reduce, or if IL-6 and/or CRP levels do not reduce by at least 50% within 48 hours, the subject is given at least one additional dose.

In some embodiments, the subject has or is suspected of having COVID-19. In some embodiments, the patient experiences an improved or normal lung function after treatment, e.g., 28 days or 60 days after treatment. In some embodiments, the patient's WHO score for COVID-19 improves (i.e., lowers) by at least 2 points after treatment, e.g. 28 days or 60 days after treatment. The WHO (World Health Organization) COVID-19 score system, for example, is defined as follows, with 0=uninfected or no viral RNA detected, 1=asymptomatic but with viral RNA detected, and with higher scores representing ever more serious symptoms: 2=symptomatic, independent; 3=symptomatic, assistance needed; 4=hospitalized, no oxygen therapy; 5=hospitalized, oxygen by simple mask or nasal prongs; 6=hospitalized, oxygen by non-invasive ventilation or high flow nasal cannula; 7=intubation and mechanical ventilation, PaO₂/FiO₂ ? 150 or SpO₂/FiO₂≥200; 8=mechanical ventilation, PaO₂/FiO₂<150 or SpO₂/FiO₂<200 or vasopressors; 9=mechanical ventilation, PaO₂/FiO₂<200 and vasopressors, dialysis, or ECMO; 10=death. The WHO score as used herein is the WHO score of the patient at the beginning of treatment with anti-IL-6 antibody (i.e. determined shortly before administration of the anti-IL-6 antibody for the first time), unless clarified otherwise (e.g., by referring to a change in score with treatment). The WHO score allows defining patient groups which benefit most from the proposed treatment. In some embodiments, the patient's WHO score at the time of anti-IL-6 antibody treatment is greater than 3. In some embodiments, the patient's WHO score at the time of anti-IL-6 antibody treatment is 4 or greater. In some further embodiments, the patient's WHO score at the time of anti-IL-6 antibody treatment is 5 or greater. A score or a score or other diagnosis “at the time of treatment” means the score or diagnosis obtained just before treatment commences, such as in order to determine whether to give treatment or what type of treatment a patient should receive. In some embodiments, the patient's WHO score is between 4 and 7. In some embodiments, the patient's WHO score is between 4 and 6. In some embodiments, the patient's WHO score is 4 or 5. In some embodiments, it is between 5 and 7. In some embodiments, the patient's WHO score is less than 8. In some embodiments, the patient has a WHO score of 7 or lower (i.e., not a score of 8 or 9). In other embodiments, the WHO score is less than 7 (i.e., 6 or lower). In other embodiments, the WHO score is less than 5 (i.e., 5 or lower).

In some embodiments, the patient has not been intubated, and/or is not on an invasive ventilator at the time that the anti-IL-6 antibody treatment is started (i.e. at the beginning of treatment with anti-IL-6 antibody). In some embodiments, the patient has been intubated, and/or is on an invasive ventilator at the time the anti-IL-6 antibody treatment is started. In some embodiments, the patient has acute lung injury, in particular with PaO₂/FiO₂>200 mmHg.

The Horowitz index (P/F ratio; PaO₂/FiO₂ ratio) is defined as the ratio of partial pressure of oxygen in blood (PaO₂), in millimeters of mercury, and the fraction of oxygen in the inhaled air (FIO₂). This diagnostic criteria for acute lung injury (ALI) and ARDS is utilized to assess the degree of hypoxemia and/or lung function of patients on ventilators. In some embodiments, the patient has a Horowitz index of less than 450 mmHg at the time when anti-IL-6 antibody treatment is started. In some embodiments, the patient has a Horowitz index of less than 350 mmHg at the time when anti-IL-6 antibody treatment is started. In some embodiments, the patient has a Horowitz index of less than 300 mmHg when anti-IL-6 antibody treatment is started. In some embodiments, the patient has a Horowitz index of more than 100 mmHg when anti-IL-6 antibody treatment is started (or of 100 to <300; mild or moderate ARDS). In some embodiments, the patient has a Horowitz index of more than 150 mmHg when anti-IL-6 antibody treatment is started (or of 150 to <300). In some embodiments, the patient has a Horowitz index of more than 200 mmHg when anti-IL-6 antibody treatment is started (or of 200 to <300; mild ARDS). In some embodiments, PaO₂/FiO₂ ranges from 150-450 mmHg at the time when anti-IL-6 antibody treatment is started. In some embodiments, PaO₂/FiO₂ ranges from 150-350 mmHg at the time when anti-IL-6 antibody treatment is started. In some embodiments, PaO₂/FiO₂ ranges from 150-300 mmHg at the time when anti-IL-6 antibody treatment is started. In some embodiments, PaO₂/FiO₂ ranges from 150-200 mmHg at the time when anti-IL-6 antibody treatment is started. In some embodiments, PaO₂/FiO₂ ranges from 200-450 mmHg at the time when anti-IL-6 antibody treatment is started. In some embodiments, PaO₂/FiO₂ ranges from 200-400 mmHg at the time when anti-IL-6 antibody treatment is started. In some embodiments, PaO₂/FiO₂ ranges from 200-350 mmHg at the time when anti-IL-6 antibody treatment is started. In some embodiments, PaO₂/FiO₂ ranges from 200-300 mmHg at the time when anti-IL-6 antibody treatment is started. As noted above, ARDS may be classified into mild, moderate and severe ARDS according to the “Berlin Definition” (Ranieri V M, et al. Acute respiratory distress syndrome: the Berlin definition. JAMA. 2012; 307(23):2526-33). In some embodiments, the patient has no ARDS, mild ARDS, or moderate ARDS when anti-IL-6 antibody treatment is started. In some embodiments, the patient has no ARDS or mild ARDS when anti-IL-6 antibody treatment is started. In some embodiments, the patient has no ARDS when anti-IL-6 antibody treatment is started and/or PaO₂/FiO₂≥300 mmHg. In some embodiments, the patient has mild ARDS when anti-IL-6 antibody treatment is started and/or PaO₂/FiO₂ ranges from 200 to <300 mmHg. In some embodiments, the patient has mild or moderate ARDS when anti-IL-6 antibody treatment is started and/or PaO₂/FiO₂ ranges from 100 to <300 mmHg. In some embodiments, the patient has no ARDS or mild ARDS, and/or a PaO₂/FiO₂ of 200 mmHg or higher, or that ranges from 200 to 450 mmHg, when anti-IL-6 antibody treatment is started and risk of progression to moderate or severe ARDS is reduced.

In some embodiments, a subject that has or is suspected of having COVID-19 is treated with clazakizumab at a dose of 10-25 mg, 10 mg, 12.5 mg, or 25 mg, or an equivalent dose by weight, wherein the subject either does not have symptoms of ARDS at time of treatment and/or wherein the subject has a WHO score of 4-7, 5-7, 4-6, 4-5, less than 6, less than 7, or less than 8. In some cases, the subject has a WHO score of 4-5. In some cases, the subject has a WHO score of 4-6. In some cases, the subject does not have moderate or severe ARDS (i.e., a PaO₂/FiO₂ of 200 mmHg or higher). In some cases, the subject has mild ARDS (i.e., a PaO₂/FiO₂ of 200 to <300 mmHg.) In some embodiments, the patient has not been intubated, and/or is not on an invasive ventilator. In some cases, the subject may have one or more symptoms of cytokine storm syndrome. In some embodiments, the subject is not receiving supplemental oxygen, while in other embodiments, the subject is receiving supplemental oxygen. In some embodiments, the subject is receiving supplemental oxygen, but is not on a mechanical ventilator and/or is not intubated, for example, the subject may be receiving non-invasive ventilation. In some embodiments, the patient has elevated levels of IL-6, such as greater than 20 pg/mL, greater than 25 pg/mL, greater than 30 pg/mL, or greater than 35 pg/mL, prior to treatment. In some embodiments, the patient has elevated C-reactive protein (CRP) prior to treatment, such as greater than 35 mg/L, greater than 50 mg/L, or greater than 100 mg/L. In some embodiments, a subject whose IL-6 and/or CRP are elevated is given one dose of anti-IL-6 antibody such as clazakizumab, and then levels of IL-6 and/or CRP are re-checked after 24-48 hours and if not decreased by at least 20%, at least 30%, at least 40%, at least 50%, at least 60%, at least 70%, at least 80%, at least 90%, then a second dose of antibody is administered, for example within 24 or 48 hours after the first dose. In some embodiments, a subject whose IL-6 and/or CRP are elevated is given one dose of anti-IL-6 antibody such as clazakizumab, and then levels of IL-6 and/or CRP are re-checked after 24-48 hours and if not decreased by at least 50%, then a second dose of antibody is administered, for example within 24 or 48 hours after the first dose. In some embodiments, only one dose of antibody is administered. In other embodiments, a second dose is administered. In yet further embodiments, more than two doses are administered.

In some embodiments, the subject has one or more conditions, such as one or more of hypertension, obesity (BMI>40), type 2 diabetes, cardiac disease other than hypertension, tobacco use, underlying pulmonary disease, chronic immunosuppression, cancer, or history of malignancy (other than skin cancer), optionally in combination with an infection such as COVID-19. In some embodiments, the subject has or is suspected of having COVID-19, and also has an SpO₂ of <90%, supplemental oxygen treatment for at least 24 hours, an IL-6 level of at least 20 pg/mL, a CRP level of >35 mg/L, a ferritin level of >500 ng/mL, a D-dimer of >1 mg/L, a neutrophil-lymphocyte ration of >4, an LDH level of >200 U/L, and/or an increase in troponin without known cardiac disease.

### Exemplary Pharmaceutical Compositions

In some embodiments, the present disclosure provides a pharmaceutical composition suitable for treating ARDS or CRDS or the symptoms associated therewith or for use in methods described herein. In some embodiments, the composition includes clazakizumab and a pharmaceutically acceptable carrier or excipient, and optionally one or more other immunosuppressants. In some embodiments, the composition includes an anti-IL-6 antibody that (a) comprises a variable light chain polypeptide comprising light chain complementarity defining region (CDRs) comprising amino acid sequences of SEQ ID NOs: 4, 5 and 6, and a heavy chain comprising heavy chain CDRs comprising amino acid sequences of SEQ ID NOs: 7, 8 or 120, and 9; (b) comprises a heavy chain comprising the amino acid sequence of SEQ ID NO: 704 or 745 and comprises a light chain comprising the amino acid sequence of SEQ ID NO: 702 or 746; or (c) comprises a heavy chain comprising the amino acid sequence of SEQ ID NO: 657 and comprises a light chain comprising the amino acid sequence of SEQ ID NO: 709; and a pharmaceutically acceptable carrier or excipient and optionally one or more other immunosuppressants.

Pharmaceutical compositions for use in methods according to the disclosure can contain any pharmaceutically acceptable excipient. Examples of excipients include but are not limited to starches, sugars, microcrystalline cellulose, diluents, granulating agents, lubricants, binders, disintegrating agents, wetting agents, emulsifiers, coloring agents, release agents, coating agents, sweetening agents, flavoring agents, perfuming agents, preservatives, antioxidants, plasticizers, gelling agents, thickeners, hardeners, setting agents, suspending agents, surfactants, humectants, carriers, stabilizers, and combinations thereof.

In various embodiments, the pharmaceutical compositions according to the disclosure may be formulated for delivery via any route of administration. This may include e.g., aerosol, nasal, oral, transmucosal, transdermal, parenteral or enteral. In some embodiments, administration is intravenous or subcutaneous.

“Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the parenteral route, the compositions may be in the form of solutions or suspensions for infusion or for injection. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Typically, the compositions are administered by injection. Methods for these administrations are known to one skilled in the art.

Pharmaceutical compositions according to the disclosure can contain any pharmaceutically acceptable carrier. For example, the carrier may be a liquid or solid filler, diluent, excipient, solvent, or encapsulating material, or a combination thereof.

### Additional Embodiments

Further exemplary embodiments of the instant disclosure include the following:
1. A method of preventing, stabilizing or reducing acute or chronic respiratory distress syndrome in a patient who comprises or is suspected of comprising a coronavirus infection comprising administering to said subject a prophylactically or therapeutically effective amount of an anti-human interleukin-6 (IL-6) antibody or anti-human IL-6 antibody fragment, wherein the antibody or antibody fragment comprises: a variable light chain polypeptide comprising the CDRs of SEQ ID NOs:4, 5 and 6 and, and a variable heavy chain polypeptide comprising the CDRs of SEQ ID NOs:7, 8 or 120, and 9.
2. The method of embodiment 1, which prevents the patient from progressing to a clinical endpoint consistent with an ARDS diagnosis or which results in a much less severe form of ARDS, i.e., which does not progress to cytokine storm, sepsis and/or organ failure.
3. The method of embodiment 1 or 2, wherein the coronavirus infection is caused by COVID-19.
4. The method of any of the previous embodiments, wherein the anti-human IL-6 antibody comprises the heavy chain polypeptide of SEQ ID NO: 704 or 745 and comprises the light chain polypeptide of SEQ ID NO: 702 or 746.
5. The method of any of the previous embodiments, wherein the patient is confirmed to be COVID-19 positive prior to treatment.
6. The method of any of the previous embodiments, wherein the patient is confirmed to be COVID-19 after starting treatment.
7. The method of any of the previous embodiments, wherein the patient shows at least one symptom of ARDS or CRDS or pulmonary problems prior to treatment.
8. The method of embodiment 6, wherein said symptoms or pulmonary problems include one or more of the following: barotrauma (volutrauma), pulmonary embolism (PE), pulmonary fibrosis, ventilator-associated pneumonia (VAP); gastrointestinal bleeding (ulcer), dysmotility, pneumoperitoneum, bacterial translocation; Hypoxic brain damage; abnormal heart rhythms, myocardial dysfunction; acute kidney failure, positive fluid balance; vascular injury, pneumothorax, tracheal injury/stenosis; malnutrition (catabolic state), electrolyte abnormalities; Atelectasis, blood clots, weakness in muscles used for breathing, stress ulcers, depression or other mental illness; single or multiple organ failure; pulmonary hypertension or increase in blood pressure in the main artery from the heart to the lungs.
9. The method of any of the previous embodiments, wherein the levels of IL-6 in the patient are detected prior to treatment.
10. The method of embodiment 9, wherein the levels of IL-6 in the patient are detected and confirmed to be elevated prior to treatment.
11. The method of any of the previous embodiments, wherein the levels of CRP in the patient are detected prior to treatment.
12. The method of embodiment 11, wherein the levels of CRP in the patient are detected and confirmed to be elevated prior to treatment.
13. The method of any of the previous embodiments, wherein IL-6 and/or CRP levels in the patient are detected during and/or after treatment.
14. The method of any of the previous embodiments, wherein the anti-IL-6 antibody or antibody fragment is administered at a dose ranging from 0.01-5000 mg, 1-1000 mg, 1-500 mg, 5 mg-50 mg or about 10 mg).
15. The method of any of the previous embodiments, wherein the anti-IL-6 antibody or antibody fragment is administered about once monthly or every 4 weeks intravenously or via subcutaneous injection.
16. The method of any of the previous embodiments, wherein the patient is administered another active or another therapeutic regimen used to treat coronavirus infection and/or treat or prevent ARDS or CRDS.
17. The method of any of the previous embodiments, wherein the patient receives another treatment for ARDS, optionally one or more of corticosteroids; inhaled nitric oxide (NO); extracorporeal membrane oxygenation (venovenous or venoarterial) or another immunosuppressive agent, optionally thymoglobulin, basiliximab, mycophenolate mofetil, tacrolimus, an anti-CD20 mAb such as rituximab, or a corticosteroid.
18. The method of any of the previous embodiments, wherein the patient is additionally treated with an antiviral or antibiotic or another immunosuppressive agent, optionally another antibody.
19. The method of any of the previous embodiments, wherein the patient has pneumonia caused by COVID-19 or caused by another pathogen, e.g., another virus, a bacterium or a fungus.
20. The method of any of the previous embodiments, wherein the patient has one or more risk factors for developing ARDS or a poor ARDS prognosis; optionally the patient is over 60, 65, 70 years of age, the patient has type 1 or type 2 diabetes, the patient has high blood pressure, the patient has cancer, the patient has an inflammatory lung condition, e.g., asthma, COPD or cystic fibrosis, the patient has arteriosclerosis, the patient has another inflammatory or autoimmune condition or a combination of any of the previous.
21. The method of any of the previous embodiments, wherein the patient is on a ventilator or respirator prior to antibody administration.
22. The method of any of the previous embodiments, wherein the patient has improved or normal lung function after antibody treatment.
23. The method of any of the previous embodiments wherein the anti-IL-6 antibody comprises human IgG1 constant regions.
24. The method of embodiment 23, wherein the human IgG1 constant regions comprise the constant light polypeptide of SEQ ID NO: 586 and the constant heavy polypeptide of SEQ ID NO: 588.
25. The method of any of the previous embodiments wherein the anti-IL-6 antibody comprises the variable heavy chain polypeptide of SEQ ID NO: 657 and the variable light chain polypeptide of SEQ ID NO: 709.
26. The method of any of the previous embodiments wherein the anti-IL-6 antibody comprises the heavy chain polypeptide of SEQ ID NO: 704 or 745 and the light chain polypeptide of SEQ ID NO: 702 or 746.
27. The method of any of the previous embodiments wherein the anti-IL-6 antibody is dosed intravenously or subcutaneously every 4 weeks or monthly.
28. The method of any of the previous embodiments wherein a 10 mg or 12.5 mg dose of the anti-IL-6 antibody is administered intravenously every 4 weeks or monthly.
29. The method of any of the previous embodiments wherein a 10 mg or 12.5 mg or 25 mg dose of clazakizumab is administered subcutaneously every 1 week, 2 weeks, 4 weeks or monthly.
30. The method of any of the previous embodiments wherein the patient optionally is further treated with any of the following:
(i) azathioprine (e.g., 1.0-2.0 mg/kg/day),
(ii) calcineurin inhibitors (CNIs),
(iii) mycophenolate mofetil (MMF) (e.g., 1.0-2.0 g/day)/mycophenolic acid (MPA) (e.g., 720-1440 mg/day),
(iv) mTOR inhibitors (e.g., tacrolimus, (e.g., target trough levels 5-8 ng/ml) everolimus, sirolimus),
(v) low dose corticosteroids (e.g., prednisone/prednisolone ≤10 mg/day),
(vi) antihypertensive agents (e.g., angiotensin converting enzyme inhibitors (ACEIs),
(vii) angiotensin II receptor blockers (ARBs),
(viii) cyclosporine, (e.g., target trough levels 50-150 ng/ml)
(ix) antidiabetogenic agents;
(x) or a combination of any of the previous.
31. The method of any of the previous embodiments wherein the patient optionally is further treated with Pneumocystis jiroveci pneumonia (PJP) prophylaxis, e.g., trimethoprim (e.g., 80 mg daily pill), and/or sulfamethoxazole (e.g., 160 mg 3 times weekly pill), inhaled pentamidine or oral dapsone (optionally commenced within at least 1 week of treatment).
32. The method of any of the previous embodiments wherein if the patient is treated with a pulse steroid such as oral prednisone, e.g., 200 mg/day).
33. The method of any of the previous embodiments wherein clazakizumab is used in combination with the standard of care immunosuppression regimens (e.g., thymoglobulin, basiliximab, mycophenolate mofetil, tacrolimus, corticosteroids).
34. The method of any of the previous embodiments wherein, wherein the anti-IL-6 antibody or antibody fragment contains an Fc region that has been modified to alter effector function, half-life, proteolysis, and/or glycosylation.
35. The method of any of the previous embodiments wherein the anti-IL-6 antibody is selected from a humanized, single chain, or chimeric antibody and the antibody fragment is selected from a Fab, Fab′, F(ab′)2, Fv, or scFv.
36. The method of any of the previous embodiments wherein the antibody dose is between about 0.001 and 100 mg/kg of body weight of recipient patient.
37. The method of any of the previous embodiments wherein the anti-IL-6 antibody dose is between about 0.1 and 20 mg/kg of body weight of recipient patient or comprises about 10-12.5 mg.
38. The method of any of the previous embodiments wherein the anti-IL-6 antibody or fragment inhibits the binding of IL-6 to gp130 and/or to IL-6R1.
39. The method of any of the previous embodiments wherein the anti-IL-6 antibody or antibody fragment comprises a human constant region.
40. The method of embodiment 39, wherein said human constant region comprises an IgG1, IgG2, IgG3 or IgG4 constant region.
41. The method of embodiment 39, wherein said human constant region comprises an IgG1 constant region.
42. The method of any of the previous embodiments wherein the anti-IL-6 antibody is clazakizumab.
43. The method of any of the previous embodiments which promotes survival patient to and/or improves or restores normal lung function.
44. The method of any of the previous embodiments which eliminates the need for the need for the patient to go on a ventilator or reduces the time the patient is on a ventilator.
45. The method of any of the previous embodiments which results in one or more of the following compared to placebo: (i) time to liberation from mechanical ventilation is reduced; (ii) time to durable fever resolution is reduced, (iii) time to durable improvement of oxygenation is reduced, (iv)C-reactive protein response is reduced, (v) length of ICU stay is reduced or (vi) number of patients who survive 28 days post treatment is increased.
46. The method of any of the previous embodiments wherein the treated COVID-19 infected patient is hospitalized but does not exhibit pulmonary or respiratory difficulties so extreme that they require exogenous high levels of oxygen.
47. The method of embodiment 46 wherein said patient is treated with either a single intravenous dose of clazakizumab 10 mg, 12.5 mg, or 25 mg of clazakizumab.
48. The method of any of the previous embodiments which (i) reduces the number of COVID-19 infected patients who develop ARDS; (ii) slows the onset of ARDS in COVID-19 infected patients and/or (iii) results in an ARDS condition in COVID-19 infected patients which is less severe than in patients not administered clazakizumab.
49. The method of any of the previous embodiments which on average results in a much less severe form of ARDS than in patients not administered clazakizumab, i.e., the ARDS condition does not progress to cytokine storm, sepsis and/or organ failure.
50. The method of any of the previous embodiments, wherein a single 25 mg intravenous dose of clazakizumab is administered to the patient and a second dose administered 24-48 h later if the C-reactive protein (CRP) fails to sharply decrease after one dose (i.e., by at least 20, 30, 40, 50, 60, 70, 80, 90% or more.
51. The method of any of the foregoing embodiments wherein the patient comprises at least one of the following: (i) is intubated mechanically ventilated with acute respiratory distress syndrome (ARDS), and requires vasopressor support; (ii) has profound hypoxemia requiring supported by non-invasive ventilationory (NIV) support; (iii) is a solid organ recipient, optionally a kidney or heart recipient; (iv) shows sign of renal failure, optionally acute renal failure; (v) has (optionally markedly) elevated IL-6; (vi) has (optionally markedly) increased higher D-dimer levels, (vii) has optionally markedly) increased fibrinogen levels and/or (viii) has increased (optionally markedly) ferritin levels; or a combination of any of the foregoing; wherein said increase( )s if present are relative to median levels observed in normal (non-inflammatory) persons.

In order to further describe embodiments of the disclosure, the following examples are provided.

### Example 1

### Use of Clazakizumab to Treat/Prevent ARDS in COVID-19 Infected Patients

### Clazakizumab Treatment Regimen

Clazakizumab will be used as a primary therapeutic in treating ARDS and symptoms thereof in COVID-19 infected patients or patients suspected to comprise COVID-19 infection. As afore-mentioned, coronaviruses, e.g., COVID-19, in severe cases, causes Acute Respiratory Distress Syndrome (ARDS), in many cases leading to death.

In ARDS, cytokine release syndrome (CRS; i.e., cytokine storm syndrome) caused by an uncontrolled immune response, unleashes the most devastating effects. Clazakizumab potently and rapidly shuts down IL-6 signaling and unlike IL-6R blocking mAbs, clazakizumab blocks IL-6 cytokine directly. It may be used to treat ARDS and symptoms associated therewith in COVID-19 infected patients in need thereof, e.g., those with high IL-6 or CRP levels and/or patients already exhibiting symptoms of ARDS such as cytokine storm and impaired lung function.

Also, since blockade of the IL-6 receptor leads to an accumulation of IL-6 cytokine, when IL-6R blocking therapeutics such as tocilizumab or sarilumab (Kevzara®, Sanofi-Regeneron) are discontinued, the release of accumulated IL-6 can potentially be detrimental. By contrast clazakizumab rapidly and effectively inactivates IL-6 cytokine in the body, therefore eliminating this potential concern.

Clazakizumab may be used to ameliorate or block the effects of the greatly increased levels of IL-6 in CRS and should effectively treat ARDS.

Another advantage of clazakizumab for use in treating ARDS in COVID-19 patients is its ability to potently and durably suppress the IL-6 response with a single, small dose.

Clazakizumab will be administered to COVID-19 patents having or at risk of developing ARDS at a target dose of 10 mg administered monthly or every 4 weeks or by intravenous or subcutaneous injection. Each 10 mg dose is administered as a 1 mL injection of clazakizumab (10 mg/mL).

### Example 2

### A Randomized Placebo-Controlled Safety and Dose-Finding Study for the Use of the IL-6 Inhibitor Clazakizumab in Patients with Life-Threatening COVID-19 Infection

Clazakizumab is administered to patients with life-threatening COVID-19 infection manifest by pulmonary failure and a clinical picture consistent with a cytokine storm syndrome. This is a single-center randomized, double-blind, placebo-controlled trial in which 30 patients will be enrolled and randomly assigned in a 1:1:1 ratio to three study arms that will receive clazakizumab at a dose of 12.5 mg, 25 mg or placebo.

The primary objective is to assess the safety of clazakizumab treatment in COVID-19 infected patients with respiratory failure due to hyperinflammation related to cytokine storm.

The secondary objectives are to assess efficacy in terms of decreasing patient mortality, shortening the duration of mechanical ventilation, and minimizing the length of ICU stay.

The primary endpoint is patient safety assessed by serious adverse events associated with clazakizumab or placebo.

The secondary endpoints are: time to liberation from mechanical ventilation; time to durable fever resolution, time to durable improvement of oxygenation, C-reactive protein response, length of ICU stay and patient survival at 28 days post treatment.

In this study clazakizumab is administered as a single dose (If second dose is necessary, administered within 48-hour period).

Thirty patients with known COVID-19 disease, male or female, 18 years of age or older, who are critically ill with respiratory failure on mechanical ventilation and do not have any of irreversible injury deemed non-survivable. The study is being conducted at Single-Center, NYU Langone Health, New York, N.Y., United States with so patients.

The patients who are randomized to the investigational arms will be given either a single intravenous dose of clazakizumab 12.5 mg, clazakizumab 25 mg or placebo. Patients who fail to achieve the expected decrease in inflammatory markers following the first dose within 48 hours will have the day 1 dose repeated (12.5 mg, clazakizumab 25 mg or placebo) on or before day 3. Clazakizumab will be administered by intravenous infusion over 30 minutes.

Placebo patients are administered normal saline infusion. Treated patients are infused with clazakizumab and blood drawn and IL-6, CRP and cytokine levels are detected.

### Example 3

### Use of the IL-6 Inhibitor Clazakizumab in Patients with Life-Threatening COVID-19 Infection

Clazakizumab is administered to patients with life-threatening COVID-19 infection. Particularly, COVID-19 infected patients with less critical disease are administered clazakizumab in a dose-ranging placebo controlled design.

Specifically, patients who are hospitalized but do not exhibit pulmonary or respiratory difficulties so extreme that they require exogenous oxygen or high levels of oxygen are treated with either a single intravenous dose of 10 mg, 12.5 mg, or 25 mg clazakizumab or a saline solution placebo.

Before and after clazakizumab administration patients will be assessed to detect levels of inflammatory markers such as IL-6, CRP and other cytokines. If following the first dose the inflammatory markers do not decrease within 48 hours of clazakizumab or placebo administration then the patients will be administered a second clazakizumab dose (10 mg, 12.5 mg, or 25 mg clazakizumab or placebo) on or before day 3. Clazakizumab and the placebo will again be administered by intravenous infusion over 30 minutes.

### Example 4

### Use of the IL-6 Inhibitor Clazakizumab to Prevent ARDS or Make ARDS Less Severe in Patients with COVID-19 Infection

As afore mentioned, a preventative therapeutic treatment regimen with Clazakizumab might prevent patients from progressing to a clinical endpoint consistent with an ARDS diagnosis or will result in a much less severe form of ARDS, i.e., which does not progress to cytokine storm, sepsis and/or organ failure.

Emerging therapeutic clinical trials for symptomatic COVID-19 patients generally fall into two categories: those testing antiviral agents, and those testing interventions aimed at mitigating the pathophysiology that ensues in in severe cases. The observation that a hyperinflammatory state accompanies the development of critical disease. We hypothesized that clazakizumab, a monoclonal antibody (mAb) directed against the pro-inflammatory cytokine interleukin-6 (IL-6) might benefit patients with respiratory failure and biochemical evidence of a cytokine storm. Toward that end we obtained single-patient emergency investigational new drug (EIND) authorizations (EIND) from the Food and Drug Administration to treat seven critically ill COVID-19 patients with a single 25 mg intravenous dose of clazakizumab. A second dose was administered 24-48 h later if the C-reactive protein (CRP) failed to sharply decrease after one dose.

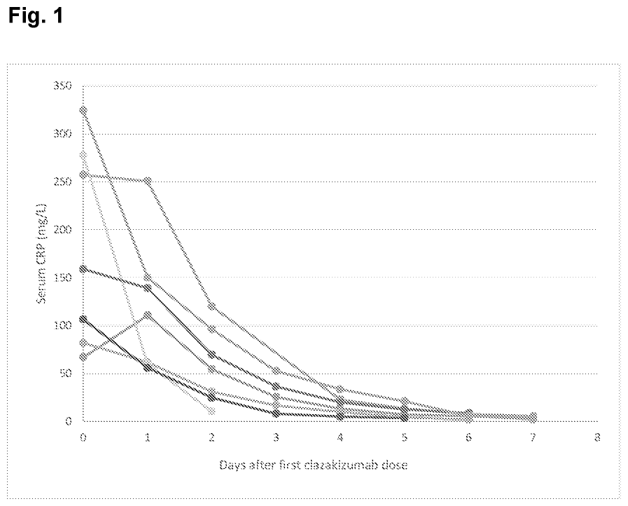

Mean patient age was 64±12 y and 6 of 7 of these patients were male. At the time of clazakizumab administration, 5 of 7 patients were intubated mechanically ventilated with acute respiratory distress syndrome (ARDS), and required vasopressor support. Two others had profound hypoxemia requiring supported by non-invasive ventilationory (NIV) support. Four had acute renal failure. Two were solid organ transplant recipients. At baseline, all had markedly elevated IL-6 (median: 121 pg/ml, IQR:35-201), D-dimer (median: 3695 ng/ml, IQR:1700-5800), fibrinogen (median: 632 mg/dl, IQR:439-831), and ferritin levels (median: 1766 ng/ml, IQR:1172-2057). Serial measurements of serum CRP declined sharply in all cases following clazakizumab administration (as shown in FIG. 1).

Of the 5 ARDS patients on mechanical ventilation, one had numerous comorbidities including chronic immunosuppression, diabetes, and coronary artery disease. Despite improved oxygenation after receiving clazakizumab, this patient progressed to multi-organ failure, shock, cardiac arrest, and expired. Three others were successfully extubated. The fourth underwent tracheostomy and was weaned off the ventilator, but remains hospitalized recovering from a perforated peptic ulcer. Intestinal perforations have been rarely observed in patients receiving anti-IL-6 mAb clazakizumab, but in the setting of critical illness, determining relatedness to clazakizumab is challenging. Two patients had aspiration events that prolonged their recoveries. Neither patient requiring NV was intubated.

These outcomes should be viewed in the context of a recent report of 5700 patients hospitalized with COVID-19 within a New York City health system, in which the mortality rate of mechanically ventilated patients was 88.1%. In our series of critically ill patients, 4 of 5 patients with overt ARDS survived to ventilator independence; comparably, a 20% mortality rate, and. Of our clazakizumab treated 7 patients, 4 have since been discharged from the hospital after receiving clazakizumab. Overall survival was 86% (6/7). Our encouraging experience implores the need to assess efficacy of this drug in a proper randomized-controlled trial.

### Example 5

Single Patient Expanded Access Trial of the IL-6 Inhibitor Clazakizumab for the Treatment of Life-Threatening COVID-19 Infection

### LIST OF ABBREVIATIONS

### AE Adverse Event/Adverse Experience

### CFR Code of Federal Regulations

### CRF Case Report Form

CRP C-reactive protein

### CSOC Clinical Study Oversight Committee

### DCC Data Coordinating Center

### DHHS Department of Health and Human Services

### DSMB Data and Safety Monitoring Board

### FFR Federal Financial Report

### FWA Federalwide Assurance

### GCP Good Clinical Practice

### HIPAA Health Insurance Portability and Accountability Act

### ICF Informed Consent Form

### ICH International Conference on Harmonisation

### IL-6 Interleukin-6

### IRB Institutional Review Board

### ISM Independent Safety Monitor

### MOP Manual of Procedures

N Number (typically refers to participants)

### NIH National Institutes of Health

### OHRP Office for Human Research Protections

### OHSR Office of Human Subjects Research

### PI Principal Investigator

### QA Quality Assurance

### QC Quality Control

sHLH Secondary hemophagocitic lymphhistiocytosis

### SAE Serious Adverse Event/Serious Adverse Experience

### SOP Standard Operating Procedure

### US United States Background Information and Scientific Rationale

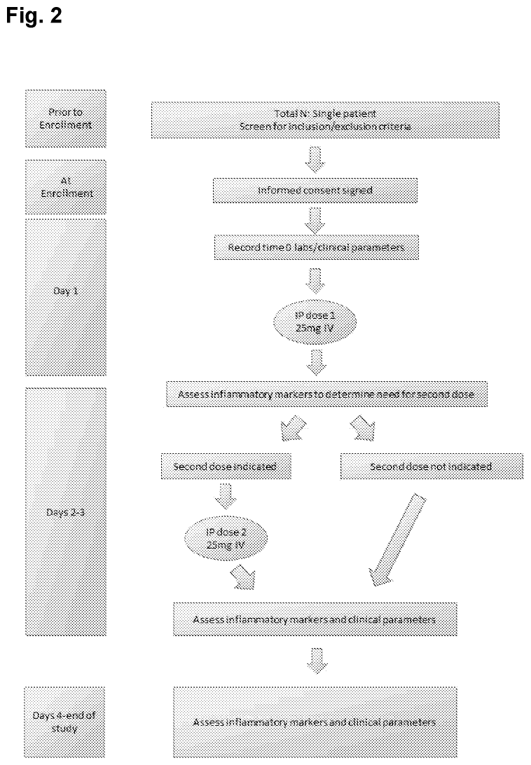

A schematic of the study design is provided in FIG. 2.

Background Information and Relevant Literature: The limited understanding of the clinical behavior of patients infected with COVID-19 is evolving on a daily basis. Reports from China indicate that a subset of patients with the worst clinical outcomes may manifest cytokine storm syndromes. Hypotheses that excess cytokines may trigger a secondary hemophagocytic lymphhistiocytosis (sHLH) have been proposed. Indeed, cytokine profiles consistent with this picture were observed in Chinese patients with severe pulmonary involvement (1). (Mehta, Lancet online and refs). Specifically, elevated ferritin and interleukin-6 (IL-6) were associated with fatalities among the infected patients. A role for targeted anti-inflammatory and anti-cytokine therapies in the treatment of pulmonary hyperinflammation has been proposed. Clazakizumab is a genetically engineered humanized IgG1 monoclonal antibody that binds with high affinity to human IL-6. This investigational agent is currently being studied as a treatment for chronic active antibody mediated rejection of renal allografts (2-4). Vitaeris holds INDs 134376 and 108525 for the ongoing clinical investigations of this agent.

Name and Description of the Investigational Agent: Clazakizumab is a genetically engineered humanized mAb directed against the human cytokine IL-6. Clazakizumab is a soluble protein consisting of 4 polypeptide chains that include 2 identical heavy chains of 450 amino acids each and 2 identical light chains of 217 amino acids each. Its molecular weight is 145,239 Daltons. It is clear to slightly opaque, colorless to yellow colored in solution. The pH in solution is 5.5-6.5.

Preclinical Data: Clazakizumab was shown to be a potent inhibitor of IL-6-induced acute phase proteins. In pharmacokinetic and pharmacodynamic studies, a single dose of clazakizumab resulted in full inhibition of IL-6 activity as measured by the inhibition of IL-6-induced phosphorylated STAT3 (pSTAT3) activity in whole blood treated ex vivo with IL-6. The results of this functional PD assay correlated with drug exposures where full inhibition of pSTAT3 activity was observed when drug levels exceeded 50 ng/mL (approximately 0.3 nM). In a tissue cross-reactivity study, tissue binding of clazakizumab was observed in multiple tissues in both human and cynomolgus monkey, was generally cytoplasmic in nature, and was consistent with the known expression of IL-6 by cells and tissues. Results from both single- and repeat-dose nonclinical toxicology studies of up to 6 months in cynomolgus monkeys demonstrated an acceptable safety profile for clazakizumab. In a preliminary enhanced pre- and post-natal development study conducted in cynomolgus monkeys, an increase in the number of monkeys with retention of the placenta at parturition was observed at clazakizumab doses of 3 mg/kg (n=2) and 30 mg/kg (n=3), corresponding to doses 34 and 340 times the planned human dose of 12.5 mg once every 4 weeks (Q4W). There were no other safety findings of clinical concern.

Clinical Data to Date: Clinical studies have been conducted in healthy subjects and in the following patient populations: RA, PsA, Crohn's disease, GVHD, and oncology. These completed clinical studies include a total of 1,223 subjects, of which 1,056 subjects were exposed to clazakizumab for up to 175 weeks (including open-label, long-term extension phases) with doses ranging from 1 mg to 640 mg, given by either IV or subcutaneous (SC) injection.

Dose Rationale: The dosing strategy is based on data from 11 completed and 3 ongoing clinical trials, interpreted in the context of the clinical problem at hand. First, regarding the route of administration, trials of both the subcutaneous and intravenous routes have been performed. Apart from the bioavailability being about 40% less by the subcutaneous route compared to intravenous, the most notable difference between the two routes is the median time to Tmax. Tmax was achieved after 1 week in patients a receiving subcutaneous dose, compared to at the end of infusion for patients receiving an intravenous dose. Given that the study subject enrolled here is critically ill, any beneficial effect of the investigational product (IP) will need to be imparted immediately. The patient may not survive long enough to see the any effect of a drug that achieves Tmax at one week. For this reason, the route of administration will be intravenous. The dose chosen was based on data from among the aforementioned clinical trials in which PK (measured in reduction in CRP) was characterized, and safety and efficacy data were reported. In these studies, a total of 1056 patients received doses ranging from 1 mg to 640 mg for up to 175 weeks. In one study clazakizumab was administered in doses of 1 mg, 5 mg, and 25 mg in groups of patients with rheumatoid arthritis. Weekly CRP levels were measured in the three groups compared to placebo. In the 25 mg group, mean CRP levels decreased most rapidly and most durably. In another study of patients with psoriatic arthritis randomized to receive placebo or doses of 25 mg, 100 mg or 200 mg clazakizumab and when comparing clinical efficacy as measured by mean change from baseline in the Psoriasis Area Severity Index score, no differences across the three dosing groups were observed. Clinically significant AEs were mainly seen at higher doses (i.e., 100 mg), whereas doses 25 mg or less were well tolerated.

### Study Rationale

As of Mar. 20, 2020, the novel 2019-coronavirus has infected nearly 250,000 people resulting in over 10,000 deaths. Treatment for this disease remains a challenge. Among the subset of patients who develop critical illness, evidence points towards the development of a cytokine storm syndrome that is similar to what is observed in secondary hemophagocytic lymphhistiocytosis (sHLH). Clinical and laboratory features of sHLH include high fevers, elevated ferritin, elevated triglycerides, low fibrinogen, and cytopenias. About half of the patients with sHLH develop ARDS which carries a high mortality (1). In China, hypercytokineamia was observed in patients with severe COVID disease and one study published online associated elevations in ferritin and IL-6 with greater mortality risk in these patients (5). It is reasonable to postulate that the pulmonary involvement may be the result of unchecked hyperinflammation, and that there may be a benefit to immunosuppressive, specifically, anti-cytokine therapies. Vitaeris Inc manufactures a direct IL-6 inhibitor, clazakizumab, which is currently under phase 3 investigations for patients with chronic active antibody mediated rejection after kidney transplantation. Recognizing, based on its mechanism of action, clazakizumb is hypothesized to have benefit for patients with life-threatening COVID-disease, Vitaeris is willing to provide drug for emergency use for patients who are at greatest risk of dying from COVID-19 disease. This study is a single-arm open label trial of the emergency use of this drug to prevent death from respiratory and multi-organ failure in COVID-19 disease.

### Potential Risks & Benefits

Known Potential Risks: Identified risks associated with clazakizumab based on experience in Phase 1-3 clinical trials include: infections, LFT abnormalities, hematologic derangements (neutropenia and thrombocytopenia), dyslipidemia, gastrointestinal perforations, injection site reactions. There are no known active metabolites of clazakizumab. Metabolism studies have not been performed for clazakizumab, which is a mAb. Metabolism studies are generally not performed for therapeutic proteins, such as mAbs, which are degraded to their component amino acids which are then recycled into other proteins.

Known Potential Benefits: We are proposing an experimental use of an agent with a similar mechanism to a drug that showed promise in a small cohort of Chinese patients who developed life-threatening pulmonary failure after acquiring COVID-19 disease. On that basis there is potential that receiving clazakizumab could rapidly abrogate the hyperinflammatory syndrome that may otherwise lead to respiratory failure and death in COVID-19 disease. Extremely limited literature exists on this topic. A recent correspondence in the Lancet summarizes the experience to date (1).

### Objectives and Purpose

Primary Objective: The primary objective is to interrupt the hyperinflammatory syndrome, thought secondary to cytokine storm that is observed in the study subject who has developed life-threating COVID-19 disease. The administration of a potent IL-6 inhibitory agent clazakizumab is hypothesized to be a lifesaving intervention in the patient with ARDS, acute pulmonary failure, who is at risk for imminent multi-organ failure and death.

Secondary Objectives: Secondary objectives will be to shorten the duration of mechanical intubation, to minimize other end-organ dysfunction (renal), and to minimize the duration of intensive care unit stay following extubation of the COVID-19 afflicted patient.

### Study Design and Endpoints

Description of Study Design: This is a single patient, open label design. We propose the emergency administration of an investigational drug in a patient with high predicted short-term mortality secondary to COVID-19 disease.

Primary Study Endpoints: The primary endpoint will be patient survival 30 days after the first administered dose of clazakizumab.

Secondary Study Endpoints: Secondary endpoints will include:
Time to extubation or separation from mechanical ventilation after administration of clazakizumab;
Duration of ICU stay after administration of clazakizumab;
Recovery of renal function (if impaired at enrollment) after administration of clazakizumab;
Kinetics of systemic inflammatory markers (CRP) after administration of clazakizumab;
Hospital length of stay after administration of clazakizumab.

### Study Enrollment and Withdrawal

Inclusion Criteria: In order to be eligible to participate in this study, the individual must meet all of the following criteria:
1) At least 18 years of age;
2) Confirmed COVID-19 disease;
3) Critically ill with respiratory failure and supported by mechanical ventilation;
4) Has a consent designee willing to provide informed consent on behalf of the patient (this assumes that a mechanically ventilated patients lacks capacity to consent on his/her own behalf.

Should it be deemed that the patient has capacity to consent, consent may be obtained from the patient.)

Exclusion Criteria: An individual who meets any of the following criteria will be excluded from participation in this study: Evidence of irreversible injury deemed non-survivable even if the pulmonary failure recovers (for example severe anoxic brain injury): Known active inflammatory bowel disease; Known active, untreated diverticulitis; Known untreated bacteremia; Pregnancy; Known hypersensitivity to the clazakizumab.

Vulnerable Subjects: The adult subject enrolled in this emergency access study will not be exclude on the basis of any vulnerable attributes. This study is designed to offer a potential lifesaving treatment to someone at risk for imminent death, and the opportunity to enroll will not be withheld provided the subject meets the above inclusion and exclusion criteria.

Strategies for Recruitment and Retention: The patient enrolled has been identified as critically ill and at risk for imminent death, despite receiving all available medical and supportive therapies.

Duration of Study Participation: The entire duration of study is expected to be 30 days. Study specific laboratory tests will be performed within the first 7 days of study initiation. Beyond 7 days, all data collected will be that which is acquired for purposes of clinical care.

Total Number of Participants and Sites: This is a single patient, single center trial which will be conducted at an NYU Langone inpatient hospital site.

Reasons for Participant Withdrawal or Termination: Death within 30 days of investigational product (IP) administration would mandate study termination. Otherwise, we would not anticipate withdrawal from the study. The IP administration will occur over a maximum of 3 days following enrollment. Study specific laboratory tests will be performed in the first week and if a patient (or consent designee should the patient lack capacity) wishes to withdraw from participation and refuse these laboratory studies, they will be free to do so. An investigator may terminate participation in the study if any clinical adverse event (AE), laboratory abnormality, or other medical condition or situation occurs such that continued participation in the study would not be in the best interest of the participant or if the participant meets an exclusion criterion (either newly developed or not previously recognized) that precludes further study participation.

Handling of Participant Withdrawals or Termination: Should a patient (or consent designee) withdraw from the study by choice, no further study specific laboratory tests will be drawn. The study team would continue to follow the patient for the primary outcome of patient survival and for secondary outcomes that would be measurable by tests and assessments done for routine clinical care. The overall clinical care of this patient will not change as a result of participation in this study nor will it change should the patient be withdrawn for any reason.

Premature Termination or Suspension of Study: This single patient study will be terminated if the patient expires prior to the 30-day end of study. The study will be terminated if the patient (or the patient's consent designee) elects to withdraw from the study prematurely.

Study Agent (Study Drug, Device, Biologic, Vaccine Etc.) and/or Procedural Intervention

Study Agent(s) and Control Description: Clazakizumab is provided as a preservative-free, ready-to-use solution for IV administration, contained in a single-dose 2-cc Type I flint glass vial that is stoppered with a 13-mm stopper and sealed with an aluminum seal. Each vial contains clazakizumab Drug Substance (12.5 mg/mL or 25 mg/mL), 25 mM histidine buffer (L-histidine, L-histidine monohydrochloride), 250 mM sorbitol, and 0.015% (w/w) polysorbate-80 at pH 6.0. An overfill is included to ensure a 1.0 mL (12.5 mg or 25 mg) withdrawable volume. In these vials, clazakizumab has a clear, colorless appearance.

Product Storage and Stability: Clazakizumab will be received as single-dose vials (12.5 mg/mL; 25 mg/mL) for injection. Clazakizumab vials should be stored at ≤−20° C.±5° C. (≤−4° F.±9° F.) with protection from light. Prepared syringes may be stored for up to 24 hours in a refrigerator, 2° C. to 8° C. (36° F. to 46° F.), and up to 4 hours of the 24 hours may be at room temperature, 15° C. to 25° C. (59° F. to 77° F.). The prepared syringes should be protected from light. Prior to administration, the prepared syringe must reach room temperature by removing from refrigeration 30 to 60 minutes before use.

Preparation of 25 mg dose from 25 mg/mL vials: To prepare the clazakizumab IP injection syringe, remove the one 25 mg/mL vial from the freezer if frozen and allow the vial to thaw at ambient temperature, protected from light, until all vial contents have liquefied (this should take approximately 15-20 minutes). During the thaw process, occasionally, gently swirl the vial. Do not attempt to speed up the warming process in any way such as using a microwave or placing the vial in warm water. Once the clazakizumab vial is at ambient temperature, remove the plastic cap on the vial and wipe the septum with the alcohol swab. Using a single use, sterile needle and syringe, to the syringe, draw up the contents of the 1 mL from the vial. Inject into a minibag containing 50 cc sterile injectable 0.9% saline. Apply a label indicating the IP clazakizumab. Transport in a light protected bag to the patient's bedside for administration.

Preparation of 25 mg dose from 12.5 mg/mL vials: To prepare the clazakizumab IP injection syringe, remove two 12.5 mg/mL vials from the freezer if frozen and allow the vials to thaw at ambient temperature, protected from light, until all vial contents have liquefied (this should take approximately 15-20 minutes). During the thaw process, occasionally, gently swirl the vials. Do not attempt to speed up the warming process in any way such as using a microwave or placing the vials in warm water. Once the clazakizumab vials are at ambient temperature, remove the plastic cap on each vial and wipe the septum with the alcohol swab. Using two single use, sterile needles and syringes, draw up 1 mL from each vial. Inject into a minibag containing 50 cc sterile injectable 0.9% saline. Apply a label indicating the IP clazakizumab. Transport in a light protected bag to the patient's bedside for administration.

Dosing and Administration: Once the patient is consented and enrolled in the trial, the first dose of 25 mg of clazakizumab will be given as soon as possible thereafter. No premedications will be given prior to the investigational product. Serum CRP will be evaluated on days 1 and 2 following clazakizumab administration to assess response. If the CRP does not decrease by 50% by day 3, a second dose of 25 mg will be given.

Route of Administration: The route of administration will be intravenous. Each dose will be administered as an infusion that is run over 30 minutes.

Starting Dose and Dose Escalation Schedule: The starting dose will be 25 mg of clazakizumab. Should the patient fail to demonstrate a 50% decrease in CRP by day 3, a second dose of 25 mg clazakizumab will be given. It is possible that a second dose, even if indicated, may be withheld if drug supply is not available at the time it is due.

Dose Adjustments/Modifications/Delays: Changes in timing between dose 1 and dose 2 (if dose 2 is determined to be indicated) might occur based on the laboratory turnaround time for the CRP test. We expect that this test will result within 24 hours of being sent. Lab reporting delays could potentially result in a delay in the administration of the second dose. In the event that the CRP lab test is delayed due to laboratory backlog, then clinical judgment of the investigators will be used to determine if a second dose is to be given. Lack of clinical improvement, in the absence of a resulted CPR test, will prompt a second dose. A second dose, if indicated by CRP levels, would be held if there was suspicion of any adverse reaction deemed likely related to the first dose.

Duration of Therapy: All investigational product (IP) administration is expected to be completed by study day 3. It may be the case that only a single dose of clazakizumab is given, in which case all IP administration will be complete after the first infusion.

Tracking of Dose: All doses will be administered to the patient in the intensive care unit. Patients will not have any responsibility for medication self-administration. Standard of care medication administration charting will be performed to ensure that the medication is administered. All critical care interventions will be performed by the clinical care team independent of the study team. These include but are not limited to: ventilator management and determination of when to extubate (or perform tracheostomy); hemodynamic support (fluid and vasoactive drug administration); mechanical circulatory support if needed (e.g. ECMO); renal replacement therapy; enteral or parenteral nutrition; surveillance for and management of infections.

Clinical Laboratory Evaluations: 1) Complete blood count with differential: hemoglobin, hematocrit, white blood cells (WBC) with differential count, platelet count; 2) Complete metabolic panel: creatinine, total bilirubin, alanine aminotransferase (ALT), aspartate; 3) Aminotransferase (AST); 4)C-reactive protein (CRP); 5) Lipid panel: total cholesterol, triglycerides; 6) Ferritin; 7) Fibrinogen; 8) Pregnancy test, to be done at screening 24 hours prior to study intervention and results must be available prior to administration of study product.

### Study Schedule

Screening: Inclusion and exclusion criteria verified; Medical history and physical exam documented; Concomitant medications documented; Calculation of baseline H-score (see attached documents) for scoring sheet
Day 1: Informed consent signed; Baseline study labs drawn; Infusion of clazakizumab 25 mg IV
Day 2: Day 2 laboratory studies; Physical examination: Collection of clinical data (hemodynamic parameters, respiratory parameters)
Day 3: Day 3 laboratory studies; Physical examination; Assessment of eligibility for repeated clazakizumab dose; Infusion of clazakizumab 25 mg IV if criteria met Collection of clinical data (hemodynamic parameters, respiratory parameters)
Day 7+/−1: Day 7 laboratory studies; Collection of clinical data (hemodynamic parameters, respiratory parameters)
Day 9+/−2: Collection of clinical data (hemodynamic parameters, respiratory parameters)
Day 14+/−2: Day 14 laboratory studies; Collection of clinical data (hemodynamic parameters, respiratory parameters)
Day 30+/−3: Collection of clinical data (hemodynamic parameters, respiratory parameters)

Additional clinical data to be collected: Date of extubation (if performed); Date of tracheostomy (if performed); Date of ICU discharge (if occurs); Date of hospital discharge (if occurs).

Withdrawal/Early Termination Visit: Should the patient choose to withdraw from the study before the 30 day study duration has elapsed, the patient's choice to withdraw will be documented. The patient will be followed by the medical record to collect clinical data obtained for standard clinical care as pertains to the study outcomes, but no further study-specific blood draws will take place.

Concomitant Medications, Treatments, and Procedures: All concomitant prescription medications being administered at enrollment and during the first 14 days of study participation will be recorded. For this protocol, a prescription medication is defined as a medication that can be prescribed only by a properly authorized/licensed clinician. Medications to be reported in the CRF are concomitant prescription medications, over-the-counter medications and non-prescription medications.

Specification of Safety Parameters: Patients will be monitored for the manifestations of adverse events related to clazakizumab infusion. Both the nature of the event and the timing related to infusion will be considered. Given the baseline laboratory and physiologic abnormalities of the patient being enrolled in this study, assessments of clinical deterioration will be difficult to attribute with certainty to the underlying disease versus the study drug infusion. The known risks associated with clazakizumab will be considered in determining likelihood of association of AEs with the drug. We will only report adverse events which are deemed more than probably related or related to the clazakizumab infusion by the Principal Investigator.

Definition of Adverse Events (AE): An adverse event (AE) is any symptom, sign, illness or experience that develops or worsens in severity during the course of the study. Intercurrent illnesses or injuries should be regarded as adverse events. Abnormal results of diagnostic procedures are considered to be adverse events if the abnormality: results in study withdrawal; is associated with a serious adverse event; is associated with clinical signs or symptoms; leads to additional treatment or to further diagnostic tests; or is considered by the investigator to be of clinical significance.

Definition of Serious Adverse Events (SAE): Adverse events are classified as serious or non-serious. A serious adverse event is any AE that is: fatal; life-threatening; requires or prolongs hospital stay; results in persistent or significant disability or incapacity; a congenital anomaly or birth defect; or an important medical event. Important medical events are those that may not be immediately life threatening, but are clearly of major clinical significance. They may jeopardize the subject, and may require intervention to prevent one of the other serious outcomes noted above. For example, drug overdose or abuse, a seizure that did not result in in-patient hospitalization, or intensive treatment of bronchospasm in an emergency department would typically be considered serious. It is important to acknowledge that the patient being enrolled in this study has a high risk of mortality at the time of enrollment in the study and there is a chance that the patient may die of COVID-19 disease despite rather than because of the clazakizumab administration. In the event of mortality, every effort will be made on the part of the investigators to assess whether there was any probable association between the study drug and the patient death. In the event of patient death, the study team will consult with the clinical critical care team in an effort to determine whether the patient death was related to the underlying clinical condition (COVID-19 infection) or whether the death was unexpected given the patient's condition between the time of study drug infusion and the patient's death.

Definition of Unanticipated Problems (UP) Involving Risk to Subjects or Others: Any incident, experience, or outcome that meets all of the following criteria: 1) unexpected in nature, severity, or frequency (i.e. not described in study-related documents): 2) related or possibly related to participation in the research (i.e. possibly related means there is a reasonable possibility that the incident experience, or outcome may have been caused by the procedures involved in the research); and 3) suggests that the research places subjects or others at greater risk of harm (including physical, psychological, economic, or social harm). This definition could include an unanticipated adverse device effect, any serious adverse effect on health or safety or any life-threatening problem or death caused by, or associated with, a device, if that effect, problem, or death was not previously identified in nature, severity, or degree of incidence in the investigational plan or application (including a supplementary plan or application), or any other unanticipated serious problem associated with a device that relates to the rights, safety, or welfare of subjects (21 CFR 812.3(s)).

Classification of an Adverse Event: The following guidelines will be used to describe severity. Mild—Events require minimal or no treatment and do not interfere with the participant's daily activities. Moderate—Events result in a low level of inconvenience or concern with the therapeutic measures. Moderate events may cause some interference with functioning. Severe —Events interrupt a participant's usual daily activity and may require systemic drug therapy or other treatment. Severe events are usually potentially life-threatening or incapacitating.

Relationship of AE to Study Agent: The clinician's assessment of an AE's relationship to study agent is part of the documentation process, but it is not a factor in determining what is or is not reported in the study. If there is any doubt as to whether a clinical observation is an AE, the event should be reported. All AEs must have their relationship to study agent assessed. In a clinical trial, the study product must always be suspect. To help assess, the following guidelines are used: Related—The AE is known to occur with the study agent, there is a reasonable possibility that the study agent caused the AE, or there is a temporal relationship between the study agent and event. Reasonable possibility means that there is evidence to suggest a causal relationship between the study agent and the AE. Not Related—There is not a reasonable possibility that the administration of the study agent caused the event, there is no temporal relationship between the study agent and event onset, or an alternate etiology has been established. For all collected AEs, the clinician who examines and evaluates the participant will determine the AE's causality based on temporal relationship and his/her clinical judgment. The degree of certainty about causality will be graded using the categories below. Definitely Related—There is clear evidence to suggest a causal relationship, and other possible contributing factors can be ruled out. The clinical event, including an abnormal laboratory test result, occurs in a plausible time relationship to drug administration and cannot be explained by concurrent disease or other drugs or chemicals. The response to withdrawal of the drug (dechallenge) should be clinically plausible. The event must be pharmacologically or phenomenologically definitive, with use of a satisfactory rechallenge procedure if necessary. Probably Related—There is evidence to suggest a causal relationship, and the influence of other factors is unlikely. The clinical event, including an abnormal laboratory test result, occurs within a reasonable time after administration of the drug, is unlikely to be attributed to concurrent disease or other drugs or chemicals, and follows a clinically reasonable response on withdrawal (dechallenge). Rechallenge information is not required to fulfill this definition. Possibly Related—There is some evidence to suggest a causal relationship (e.g., the event occurred within a reasonable time after administration of the trial medication). However, other factors may have contributed to the event (e.g., the participant's clinical condition, other concomitant events). Although an AE may rate only as “possibly related” soon after discovery, it can be flagged as requiring more information and later be upgraded to “probably related” or “definitely related,” as appropriate. Unlikely to be related—A clinical event, including an abnormal laboratory test result, whose temporal relationship to drug administration makes a causal relationship improbable (e.g., the event did not occur within a reasonable time after administration of the trial medication) and in which other drugs or chemicals or underlying disease provides plausible explanations (e.g., the participant's clinical condition, other concomitant treatments). Not Related—The AE is completely independent of study drug administration, and/or evidence exists that the event is definitely related to another etiology. There must be an alternative, definitive etiology documented by the clinician.

Expectedness: The PI will be responsible for determining whether an AE is expected or unexpected. An AE will be considered unexpected if the nature, severity, or frequency of the event is not consistent with the risk information.

Time Period and Frequency for Event Assessment and Follow-UP: The occurrence of an AE or SAE may come to the attention of study personnel during study visits and interviews of a study participant presenting for medical care, or upon review by a study monitor. All AEs including local and systemic reactions not meeting the criteria for SAEs will be captured on the appropriate RF. Information to be collected includes event description, date and time of onset, clinician's assessment of severity, relationship to study product (assessed only by those with the training and authority to make a diagnosis), and time of resolution/stabilization of the event. All AEs occurring while on study must be documented appropriately regardless of relationship. All AEs will be followed to adequate resolution. Any medical condition that is present at the time that the participant is screened will be considered as baseline and not reported as an AE. However, if the study participant's condition deteriorates at any time during the study, it will be recorded as an AE. Unanticipated problems will be recorded in the data collection system throughout the study. Changes in the severity of an AE will be documented to allow an assessment of the duration of the event at each level of severity to be performed. AEs characterized as intermittent require documentation of onset and duration of each episode. The PI will record all reportable events with start dates occurring any time after informed consent is obtained until 7 (for non-serious AEs) or 30 days (for SAEs) after the last day of study participation. At each study visit, the investigator will inquire about the occurrence of AE/SAEs since the last visit. Events will be followed for outcome information until resolution or stabilization. All unresolved adverse events should be followed by the investigator until the events are resolved, the subject is lost to follow-up, or the adverse event is otherwise explained. At the last scheduled visit, the investigator should instruct each subject to report any subsequent event(s) that the subject, or the subject's personal physician, believes might reasonably be related to participation in this study. The investigator should notify the study sponsor of any death or adverse event occurring at any time after a subject has discontinued or terminated study participation that may reasonably be related to this study. The sponsor should also be notified if the investigator should become aware of the development of cancer or of a congenital anomaly in a subsequently conceived offspring of a subject that has participated in this study.

### REFERENCES FOR EXAMPLE 5

1. Mehta P et al (2020). COVID-19: consider cytokine storm syndromes and immunosuppression. The Lancet. March 28; 395(10229):1033-1034. Published online. Available at: https://www.thelancet.com/journals/lancet/article/PIIS0140-6736(20)30628-0/fulltext
2. Karkhur S et al (2019). Interleukin-6 inhibition in the management of non-infectious uveitis and beyond. J Ophthalmic Inflamm Infect. 2019 Sep. 16; 9(1):17.
3. Weinblatt M E et al (2015). The Efficacy and Safety of Subcutaneous Clazakizumab in Patients With Moderate-To-Severe Rheumatoid Arthritis and an Inadequate Response to Methotrexate: Results From a Multinational, Phase IIb, Randomized, Double-Blind, Placebo/Active-Controlled, Dose-Ranging Study. Arthritis Rheumatol. 2015 October; 67(10):2591-600.
4. Eskandary F et al (2019). Clazakizumab in late antibody-mediated rejection: study protocol of a randomized controlled pilot trial. Trials. January 11; 20(1):37.
5. Ruan Q et al (2020) Clinical predictors of mortality due to COVID-19 based on an analysis of data of 150 patients from Wuhan, China. Intensive Care Med. 2020 Mar. 3.Epub. doi: 10.1007/s00134-020-05991-x

H-score calculator

Number of points

Temperature

| | |
|---|---|
| <38.4° C. | 0 |
| 38.4-39.4° C. | 33 |
| >39.4° C. | 49 |

Organomegaly

| | |
|---|---|
| None | 0 |
| Hepatomegaly or splenomegaly | 23 |
| Hepatomegaly and splenomegaly | 38 |

Number of cytopenias*

| | |
|---|---|
| One lineage | 0 |
| Two lineages | 24 |
| Thee lineages | 34 |

Triglycerides (mmol/L)

| | |
|---|---|
| <1.5 mmol/L | 0 |
| 1.5-4.0 mmol/L | 44 |
| >4.0 mmol/L | 64 |

Fibrinogen (g/L)

| | |
|---|---|
| >2.5 g/L | 0 |
| ≤2.5 g/L | 30 |

Ferritin ng/ml

| | |
|---|---|
| <2000 ng/ml | 0 |
| 2000-6000 ng/ml | 35 |
| >6000 ng/ml | 50 |

Serum aspartate aminotransferase

| | |
|---|---|
| <30 IU/L | 0 |
| ≥30 IU/L | 19 |

Haemophagocytosis on bone marrow aspirate

| | |
|---|---|
| No | 0 |
| Yes | 35 |

Known immunosuppression†

| | |
|---|---|
| No | 0 |
| Yes | 18 |

The HScore generates a probability for the presence of secondary HLH. HScores greater than 169 are 93% sensitive and 86% specific for HLH. Note that bone marrow haemophagocytosis is not mandatory for a diagnosis of HLH. HScores can be calculated using an online HScore calculator.
HLH = haemophagocytic lymphohistiocytosis.
*Defined as either haemoglobin concentration of 9.2 g/dL or less (≤5.71 mmol/L), a white blood cell count of 5000 white blood cells per mm³ or less, or platelet count of 110000 platelets per mm³ or less, or all of these criteria combined.
†HIV positive or receiving long-term immunosuppressive therapy (ie, glucocorticoids, cyclosporine, azathioprine).

The above table is adopted from Mehta et al., Lancet. 2020; 395(10229): 1033-1034. The H-score can also be calculated using an online calculator found at http://saintantoine.aphp.fr/score/

Schedule of Events

| | | Day | Day | Day | Day | Day | Day |
|---|---|---|---|---|---|---|---|
| Activity | Screening | 1 | 2 | 3 | 7 +/− 1 | 14 +/− 2 | 30 +/− 3 |
| Study team procedures | | | | | | | |
| Assess inclusion/exclusion criteria | X | | | | | | |
| Medical History | X | | | | | | |
| Physical Exam | X | X | X | X | | | |
| Height | X | | | | | | |
| Weight | X | | | | | | |
| Vitals signs | X | X | X | X | X | X | X |
| Consent obtained | X | | | | | | |
| Calculation of enrollment H-score | | X | | | | | |
| Dose 1 administered | | X | | | | | |
| Dose 2 administered if indicated | | | | X | | | |
| Laboratory Assessments | | | | | | | |
| Comprehensive chemistry panel | X | | | | X | X | |
| CBC with differential | X | | | | X | X | |
| CRP | X | X | X | X | X | X | |
| Total cholesterol | X | | | | X | X | |
| Triglycerides | X | | | | X | X | |
| Ferritin | X | | | | X | X | |
| Fibrinogen | X | | | | X | X | |

### Example 6

Randomized, Placebo-Controlled Safety and Dose-Finding Study for the Use of the IL-6 Inhibitor Clazakizumab in Patients with Life-Threatening COVID-19 Infection

### LIST OF ABBREVIATIONS

### AE Adverse Event/Adverse Experience

### ARDS Acute Respiratory Distress Syndrome

### CFR Code of Federal Regulations

### CRF Case Report Form

CRP C-reactive protein

### CSOC Clinical Study Oversight Committee

### DCC Data Coordinating Center

### DHHS Department of Health and Human Services

### DSMB Data and Safety Monitoring Board

### FFR Federal Financial Report

FWA Federal wide Assurance

### GCP Good Clinical Practice

### HIPAA Health Insurance Portability and Accountability Act

### ICF Informed Consent Form

### ICH International Conference on Harmonisation

### IL-6 Interleukin-6

### IRB Institutional Review Board

### ISM Independent Safety Monitor

### MOP Manual of Procedures

N Number (typically refers to participants)

### NIH National Institutes of Health

### OHRP Office for Human Research Protections

### OHSR Office of Human Subjects Research

### PI Principal Investigator

### QA Quality Assurance

### QC Quality Control

sHLH Secondary hemophagocitic lymphhistiocytosis

### SAE Serious Adverse Event/Serious Adverse Experience

### SOP Standard Operating Procedure

### US United States

### Introduction, Background Information and Scientific Rationale

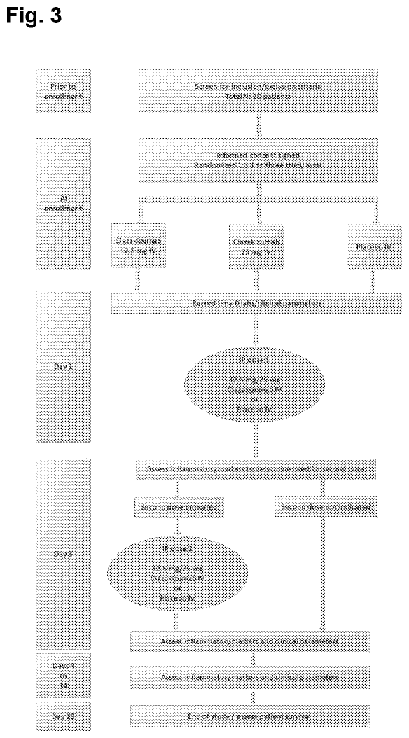

A schematic of the study design is provided in FIG. 3.

Background Information and Relevant Literature: The limited understanding of the clinical behavior of patients infected with COVID-19 is evolving on a daily basis. Reports from China indicate that a subset of patients with the worst clinical outcomes may manifest cytokine storm syndrome. Hypotheses that excess cytokines may trigger a secondary hemophagocytic lymphhistiocytosis (sHLH) have been proposed. Indeed, cytokine profiles consistent with this picture were observed in Chinese patients with severe pulmonary involvement (1). Specifically, elevated ferritin and interleukin-6 (IL-6) were associated with fatalities among the infected patients. A role for targeted anti-inflammatory and anti-cytokine therapies in the treatment of pulmonary hyperinflammation has been proposed. Clazakizumab is a genetically engineered humanized IgG1 monoclonal antibody (mAb) that binds with high affinity to human IL-6. This investigational agent is currently being studied as a treatment for chronic active antibody mediated rejection of renal allografts (2-4). In this study we propose to administer clazakizumab to patients with life-threatening pulmonary failure secondary to COVID-19 infection.

Name and Description of the Investigational Agent: Clazakizumab is a genetically engineered humanized mAb directed against the human cytokine IL-6. Clazakizumab is a soluble protein consisting of 4 polypeptide chains that include 2 identical heavy chains of 450 amino acids each and 2 identical light chains of 217 amino acids each. Its molecular weight is 145,239 Daltons. It is clear to slightly opaque, colorless to yellow colored in solution. The pH in solution is 5.5-6.5.

Preclinical Data: Clazakizumab was shown to be a potent inhibitor of IL-6-induced acute phase proteins. In pharmacokinetic and pharmacodynamic (PD) studies, a single dose of clazakizumab resulted in full inhibition of IL-6 activity as measured by the inhibition of IL-6-induced phosphorylated STAT3 (pSTAT3) activity in whole blood treated ex vivo with IL-6. The results of this functional PD assay correlated with drug exposures where full inhibition of pSTAT3 activity was observed when drug levels exceeded 50 ng/mL (approximately 0.3 nM). In a tissue cross-reactivity study, tissue binding of clazakizumab was observed in multiple tissues in both human and cynomolgus monkey, was generally cytoplasmic in nature, and was consistent with the known expression of IL-6 by cells and tissues. Results from both single- and repeat-dose nonclinical toxicology studies of up to 6 months in cynomolgus monkeys demonstrated an acceptable safety profile for clazakizumab. In a preliminary enhanced pre- and post-natal development study conducted in cynomolgus monkeys, an increase in the number of monkeys with retention of the placenta at parturition was observed at clazakizumab doses of 3 mg/kg (n=2) and 30 mg/kg (n=3), corresponding to doses 34 and 340 a human dose of 12.5 mg once every 4 weeks (Q4W). There were no other safety findings of clinical concern.

Clinical Data to Date: Clinical studies have been conducted in healthy subjects and in the following patient populations: rheumatoid arthritis, psoriatic arthritis, Crohn's disease, graft-versus-host disease, and oncology. These completed clinical studies include a total of 1,223 subjects, of which 1,056 subjects were exposed to clazakizumab for up to 175 weeks (including open-label, long-term extension phases) with doses ranging from 1 mg to 640 mg, given by either IV or subcutaneous (SC) injection. In addition, preliminary safety data are available from the ongoing pivotal Study VKTX01 (IMAGINE) in renal transplant recipients with chronic active antibody-mediated rejection and 3 ongoing Investigator-initiated trials (IITs) in the kidney transplant setting including highly-human leukocyte antigen (HLA)-sensitized subjects awaiting a kidney transplant; subjects with CABMR; and subjects with late, active ABMR.

Dose Rationale: The proposed doses of 25 mg and 12.5 mg IV in the planned COVID-19 infection trial are based on a rational dose justification taking into account the results of the clazakizumab nonclinical program, the safety and efficacy data from completed clinical trials where repeat dosing was studied, preliminary safety results from the ongoing pivotal study VKTX01 and 3 ongoing IITs in the kidney transplant setting. The completed clinical trials provide an extensive drug exposure experience to define the safety profile of clazakizumab, which is primarily associated with its IL-6 blocking effects. An intravenous route of administration is proposed for this study. Apart from the bioavailability being about 40% less by the subcutaneous route compared to intravenous, the most notable difference between the two routes is the median time to Tmax. Tmax was achieved after 1 week in patients a receiving subcutaneous dose, compared to at the end of infusion for patients receiving an intravenous dose. Given that the study subject enrolled here is critically ill, any beneficial effect of the investigational product (IP) will need to be imparted immediately. The patient may not survive long enough to see the any effect of a drug that achieves Tmax at one week. For this reason, the route of administration will be intravenous.

### Study Rationale

As of Mar. 20, 2020, the novel 2019-coronavirus has infected nearly 250,000 people resulting in over 10,000 deaths. Treatment for this disease remains a challenge. Among the subset of patients who develop critical illness, evidence points towards the development of a cytokine storm syndrome that is similar to what is observed in secondary hemophagocytic lymphhistiocytosis (sHLH). Clinical and laboratory features of sHLH include high fevers, elevated ferritin, elevated triglycerides, low fibrinogen, and cytopenias. About half of the patients with sHLH develop ARDS which carries a high mortality (1). In China, hypercytokinemia was observed in patients with severe COVID disease and one study published online associated elevations in ferritin and IL-6 with greater mortality risk in these patients (5). It is reasonable to postulate that the pulmonary involvement may be the result of unchecked hyperinflammation, and that there may be a benefit to immunosuppressive, specifically, anti-cytokine therapies. Vitaeris Inc manufactures a direct IL-6 inhibitor, clazakizumab, which is currently under phase 3 investigations for patients with chronic active antibody mediated rejection after kidney transplantation. Recognizing, based on its mechanism of action, clazakizumab is hypothesized to have benefit for patients with life-threatening COVID-disease, Vitaeris is willing to provide drug for this investigator initiated trial for use in patients who are at greatest risk of dying from COVID-19 disease. This study is a prospective, randomized, double-blind, placebo-controlled trial of clazakizumab to prevent death from respiratory and multi-organ failure in COVID-19 disease.

### Potential Risks & Benefits

Known Potential Risks: Identified risks associated with clazakizumab based on experience in Phase 1-3 clinical trials include: infections, liver function test abnormalities, hematologic derangements (neutropenia and thrombocytopenia), dyslipidemia, gastrointestinal perforations, injection site reactions. There are no known active metabolites of clazakizumab.

Metabolism studies have not been performed for clazakizumab, which is a mAb. Metabolism studies are generally not performed for therapeutic proteins, such as mAbs, which are degraded to their component amino acids which are then recycled into other proteins Since it is an immunoglobulin, no formal drug-drug interaction studies have been performed.

Known Potential Benefits: We are proposing an experimental use of an agent with a similar mechanism to a drug that showed promise in a small cohort of Chinese patients who developed life-threatening pulmonary failure after acquiring COVID-19 disease. On that basis, there is potential that receiving clazakizumab could rapidly abrogate the hyperinflammatory syndrome that may otherwise lead to respiratory failure and death in COVID-19 disease.

Extremely limited literature exists on this topic. A recent correspondence in the Lancet summarizes the experience to date (1).

### Objectives and Purpose

Primary Objective: The primary objective is to assess the safety of clazakizumab treatment in COVID-19 infected patients with respiratory failure due to hyperinflammation related to cytokine storm.

Secondary Objectives: The secondary objectives are to assess efficacy by evaluating the duration of mechanical ventilation, the length of intensive care unit (ICU) stay and patient survival in patients who receive investigational product (IP) at two different doses versus placebo.

### Study Design and Endpoints

Description of Study Design: This is a randomized, double-blind, placebo-controlled, design. We propose the administration of an investigational drug in patients with high predicted short-term mortality secondary to COVID-19 disease. 30 Patients will be randomly assigned in a 1:1:1 ratio to three study arms that will receive clazakizumab at a dose of 12.5 mg, 25 mg or placebo.

Primary Study Endpoints: The primary endpoint is patient safety assessed by serious adverse events associated with clazakizumab or placebo.

Secondary Study Endpoints: The secondary endpoints are: incidence of intubation, time to extubation, length of ICU stay, trend in C-reactive protein, and patient survival at 28 days.

### Study Enrollment and Withdrawal

Inclusion Criteria: In order to be eligible to participate in this study, the patients must meet all of the following criteria: 1) at least 18 years of age; 2) confirmed COVID-19 disease; 3) respiratory failure manifesting as: Acute Respiratory Distress Syndrome (defined in Example 6 by a P/F ratio of <100), OR SpO₂<90% on 4 L OR increasing 02 requirements over 24 hours, PLUS 2 or more of the following predictors for severe disease: CRP>35 mg/L; Ferritin>500 ng/mL; D-dimer>1 mcg/L; Neutrophil-Lymphocyte Ratio>4; LDH>200 U/L; 4) Increase in troponin in patient w/out known cardiac disease; 5) Has a consent designee willing to provide informed consent on behalf of the patient (this assumes that a mechanically ventilated patients lacks capacity to consent on his/her own behalf. Should it be deemed that the patient has capacity to consent, consent may be obtained from the patient.)

Exclusion Criteria: An individual who meets any of the following criteria will be excluded from participation in this study: 1) Evidence of irreversible injury deemed non-survivable even if the pulmonary failure recovers (for example severe anoxic brain injury); 2) Known active inflammatory bowel disease; 3) Known active, untreated diverticulitis; 4) Known untreated bacteremia; 5) Pregnancy; 6) Known hypersensitivity to the clazakizumab.

Vulnerable Subjects: Vulnerable subjects will not be excluded. This study is designed include any patients deemed at risk for imminent death, and the opportunity to enroll will not be withheld provided the subject meets the above inclusion and exclusion criteria.

Strategies for Recruitment and Retention: The patients enrolled have been identified as critically ill and failing all available medical and supportive therapies.

Duration of Study Participation: The entire duration of study participation is 28 days. Study specific laboratory tests will be performed within the first 7 days of study initiation. Beyond 7 days, all data collected will be that which is acquired for purposes of clinical care. Patients will be followed for the survival endpoint for 28 days.

Total Number of Participants and Sites: This is a single center trial which will be conducted at any NYU Langone inpatient hospital site. The anticipated enrollment is 30 total patients across the 3 arms.

Reasons for Participant Withdrawal or Termination: The predicted mortality rate for patients with COVID-19 and ARDS is in excess of 50% (6). As such we anticipate there will be mortality amongst some of the participants. Death will constitute withdrawal from the trial. Otherwise we would not anticipate premature withdrawal from the study as the patients are expected to remain hospitalized (likely in an ICU setting) for the entire duration of the 14-day period where study specific data are collected. If the patient is discharged before day 28 survival status will be obtained by patient lookup in the electronic medical record. For discharged patients in whom there is no definitive record documenting patient status (alive or dead) after 28 days have elapsed, the study team will contact the patient or patient's family by phone. The IP administration will occur over a maximum of 3 days following enrollment. Study specific laboratory tests will be performed in the first week and if a patient (or consent designee should the patient lack capacity) wishes to withdraw from participation and refuse these laboratory studies, they will be free to do so. An investigator may terminate participation in the study if any clinical adverse event (AE), laboratory abnormality, or other medical condition or situation occurs such that continued participation in the study would not be in the best interest of the participant or if the participant meets an exclusion criterion (either newly developed or not previously recognized) that precludes further study participation.

Handling of Participant Withdrawals or Termination: Should a patient (or consent designee) withdraw from the study by choice, no further study specific laboratory tests will be drawn. The study team would continue to follow the patient for the primary outcome of patient survival and for secondary outcomes that would be measurable by tests and assessments done for routine clinical care. The overall clinical care of this patient will not change as a result of participation in this study nor will it change should the patient be withdrawn for any reason.

Premature Termination or Suspension of Study: Patient deaths are expected among study subjects given that the expected mortality of the enrolling patient population exceeds 50%. All deaths and related SAEs within the study period will be reviewed by the DSMB (see section 9.8, Safety Monitoring). The DSMB will determine whether unblinding of the deceased subject is warranted. The decision to stop or suspend the study will be made the DSMB after considering the totality of the data and the benefit-risk of continuing the study.

Study Agent (Study drug, device, biologic, vaccine etc.) and/or Procedural Intervention

Study Agent(s) and Control Description: Clazakizumab is provided as a preservative-free solution for IV administration, contained in a single-dose 2-cc Type I flint glass vial that is stoppered with a 13-mm stopper and sealed with an aluminum seal. Each vial contains clazakizumab Drug Substance (12.5 mg/mL or 25 mg/mL), 25 mM histidine buffer (L-histidine, L-histidine monohydrochloride), 250 mM sorbitol, and 0.015% (w/w) polysorbate-80 at pH 6.0. An overfill is included to ensure a 1.0 mL (12.5 mg or 25 mg) withdrawable volume. In these vials, clazakizumab has a clear, colorless appearance. Placebo will be sourced locally at the study site from commercially available saline. The placebo will contain 0.9% sodium chloride as a sterile solution. There are no excipients.

Formulation, Appearance, Packaging, and Labeling: The product will be provided as kits containing two single-use glass vials of clazakizumab (12.5 mg/mL or 25 mg/mL). Vials are 2 mL, containing a minimum of 1.1 mL clazakizumab to deliver 1 mL. The placebo will contain 0.9% sodium chloride as a sterile solution and will be sourced locally by the study site.

Product Storage and Stability: Clazakizumab vials should be stored at ≤−20° C.±5° C. (≤−4° F.±9° F.) with protection from light. Prepared infusion bags may be stored for up to 12 hours in a refrigerator, 2° C. to 8° C. (36° F. to 46° F.), or at controlled room temperature, 15° C. to 25° C. (59° F. to 77° F.) and should be protected from light. The placebo should be stored at conditions specified in the product labeling.

Preparation: Investigational drug (clazakizumab or placebo) will be prepared and dispensed in identical infusion bags by an unblinded pharmacist/qualified personnel at the investigational site. Each infusion bag will contain a label with details including protocol number, subject ID, and date dispensed. The pharmacist will record the vial number dispensed for each subject, including the date and time of dispensing on an accountability log.

Preparation of 25 mg dose from 25 mg/mL vials: To prepare the clazakizumab IP solution for infusion, remove the one 25 mg/mL vial from the freezer and allow the vial to thaw at ambient temperature, protected from light, until all vial contents have liquefied (this should take approximately 15-20 minutes). During the thaw process, occasionally, gently swirl the vial. Do not attempt to speed up the warming process in any way such as using a microwave or placing the vial in warm water. Once the clazakizumab vial is at ambient temperature, remove the plastic cap on the vial and wipe the septum as well as the minibag port with the alcohol swab. Using a single use, sterile needle and syringe, draw up 1 mL of the contents from the vial. Inject into a minibag containing 50 mL sterile injectable 0.9% sodium chloride. Apply investigational study label. Transport in a light protected bag to the patient's bedside for administration.

Preparation of 12.5 mg dose from 12.5 mg/mL vials: To prepare the clazakizumab IP solution for infusion, remove the one 12.5 mg/mL vial from the freezer and allow the vial to thaw at ambient temperature, protected from light, until all vial contents have liquefied (this should take approximately 15-20 minutes). During the thaw process, occasionally, gently swirl the vial. Do not attempt to speed up the warming process in any way such as using a microwave or placing the vial in warm water. Once the clazakizumab vial is at ambient temperature, remove the plastic cap on the vial and wipe the septum as well as the minibag port with the alcohol swab. Using a single use, sterile needle and syringe, to the syringe, draw up 1 mL of the contents from the vial. Inject into a minibag containing 50 mL sterile injectable 0.9% sodium chloride. Apply investigational study label. Transport in a light protected bag to the patient's bedside for administration.

Preparation of Placebo: To prepare the placebo solution for infusion, remove one vial of 0.9% sodium chloride sterile solution from local supply at the study site. In order to be consistent with preparation of the IP solution, wait 15-20 minutes before preparing. Then remove the plastic cap on the vial and wipe the septum as well as the minibag port with the alcohol swab. Using a single use, sterile needle and syringe, to the syringe, draw up 1 mL of the contents from the vial. Inject into a minibag containing 50 mL sterile injectable 0.9% sodium chloride. Apply investigational study label. Transport in a light protected bag to the patient's bedside for administration.

Dosing and Administration: Once the patient is consented and enrolled in the trial, the first dose of 12.5 mg or 25 mg of clazakizumab or placebo will be given as soon as possible thereafter. No premedications will be given prior to the investigational product. Serum CRP will be evaluated at baseline and on days 1 and 2 following clazakizumab or placebo administration to assess response. If the CRP does not decrease by 50% within 36-48 hours after the first dose, a second dose of 12.5 mg or 25 mg clazakizumab or placebo (an identical dose to the day 1 dose) will be given no later than day 3. All doses will be administered in a blinded fashion.

Route of Administration: The route of administration will be intravenous. Each dose will be administered as an infusion that is run over 30 minutes.

Starting Dose and Dose Escalation Schedule: In the treatment arm, the starting dose will be 12.5 mg or 25 mg of clazakizumab. Should the patient fail to demonstrate a 50% decrease in CRP with 2-3 days, a second dose of 12.5 mg or 25 mg clazakizumab will be given. Clinical assessment of inflammatory parameters (fever) and assessment of CRP will be used to determine whether a second dose is administered. Patients whose CRP decreases by 50% within 2-3 days of the first dose will not be redosed. Patients with persistent fevers, and CRP failing to decrease will receive a second dose, identical to the first dose (either 12.5 mg clazakizumab, 25 mg clazakizumab or placebo) on or prior to day 3. All doses will be administered in a blinded fashion.

Dose Adjustments/Modifications/Delays: Changes in timing between dose 1 and dose 2 (if dose 2 is determined to be indicated) might occur based on the laboratory turnaround time for the CRP test. We expect that this test will result within 24 hours of being sent. Lab reporting delays could potentially result in a delay in the administration of the second dose. In the event that the CRP lab test is delayed due to laboratory backlog, then clinical judgment of the investigators will be used to determine if a second dose is to be given. Lack of clinical improvement, in the absence of a resulted CRP test, will prompt a second dose. Furthermore, the clinical assessment of inflammatory state including presence of persistent fevers will weigh into the decision for a repeat dose. A second dose, even if deemed indicated, would be held if there was suspicion of any serious adverse reaction deemed likely related to the first dose.

Duration of Therapy: All investigational product (IP) administration is expected to be completed within 48 hours of the first dose. It may be the case that only a single dose of clazakizumab is given, in which case all IP administration will be complete after the first infusion.

Tracking of Dose: All doses will be administered in the intensive care unit. Patients will not have any responsibility for medication self-administration. Standard of care medication administration charting will be performed to ensure that the medication is administered.

### Randomization, Blinding and Unblinding Procedures

Randomization: All enrolled subjects will be assigned a unique subject number, and the Investigator will maintain a list of subject numbers and subject names. A total of 30 subjects will be randomized (via an IRT) 1:1:1 into the 3 treatment arms using a stratified block randomization scheme: 10 subjects in the clazakizumab 12.5 mg group, 10 subjects in the clazakizumab 25 mg group and 10 subjects in the placebo group. The randomization schema will be pre-prepared by a statistician outside of the study team and will be made available only to the investigational pharmacy. All study investigators and clinical staff administering study drug will be blinded to the content of the dose.

Blinding: This study is double-blind and therefore neither the Investigator, the subject, the Sponsor and its representatives, nor other designated study site personnel involved in running of the study will be aware of the identification of the investigational drug administered to each subject. To maintain blinding, interim analyses will be conducted by the designated independent study monitor. Detailed procedures for maintaining the blind are specified below. Given that clazakizumab and placebo are packaged differently, investigational drug will be prepared and dispensed by an unblinded pharmacist/qualified personnel at each investigational site. To maintain blinding during the study, the pharmacist/designated staff will dispense either clazakizumab or placebo into identical infusion bags, according to each subject's randomized treatment allocation, and all subjects will receive each dose of investigational drug (clazakizumab or placebo) as an intravenous infusion. The pharmacist/designated staff will ensure that blinded personnel will not have access to drug supply records.

Unblindinq: In the event that an AE occurs for which knowledge of the identity of the investigational drug administered is necessary to manage the subject's condition and/or for regulatory reporting of a suspected unexpected serious adverse reaction (SUSAR), the blinding code for that subject may be broken by the Investigator and the treatment identified. Should emergency unblinding be required, the Investigator should call and discuss the patient with the DSMB chair before unblinding wherever possible; however, the Investigator is responsible for the medical care of the individual trial subject and does not require the agreement of the DSMB Chair before unblinding. The reason for unblinding must be documented. The information on investigational drug should only be used for decision making in the subject's further treatment. Details on unblinded treatment assignments should not be shared with the site personnel, or project team unless necessary for care of the subjects. In case of an emergency, the following process will occur after the DSMB Chair is made aware, if time permits. The Investigator will notify the unblinded Investigational Pharmacist who will serve as the source for emergent unblinding information. The Investigational Pharmacist will be responsible for the following: 1) providing unblinding information to the Investigator only for the specific Subject affected, 2) documenting the provision of unblinding information in the pharmacy records and 3) confirming with the DSMB Chair of the unblinding occurrence and rationale for unblinding.

Standard of Care Study Procedures: All critical care interventions will be performed by the clinical care team independent of the study team. These include but are not limited to: ventilator management and determination of when to extubate (or perform tracheostomy); hemodynamic support (fluid and vasoactive drug administration); Mechanical circulatory support if needed (e.g. ECMO); renal replacement therapy; enteral or parenteral nutrition and surveillance for and management of infections.

Clinical Laboratory Evaluations: 1) Complete blood count with differential: hemoglobin, hematocrit, white blood cells (WBC) with differential count, platelet count; 2) Complete metabolic panel: creatinine, total bilirubin, alanine aminotransferase (ALT), aspartate; 3) Aminotransferase (AST); 4)C-reactive protein (CRP); 5) Interleukin 6 (IL-6); 6) Lipid panel: total cholesterol, triglycerides; 7) Ferritin; 8) Fibrinogen; 9) Pregnancy test, to be done at screening 24 hours prior to study intervention and results must be available prior to administration of study product; 10) D-Dimer; 11) LDH; and 12) Troponin.

### Study Schedule

Screening: inclusion and exclusion criteria verified; informed consent signed; medical history and physical exam documented (physical examination as conducted by subject's clinical care team); concomitant medications documented; calculation of baseline H-score (see attached documents) for scoring sheet; baseline study labs drawn; collection of clinical data (vital signs, respiratory and hemodynamic support parameters)
Day 1: physical examination (as conducted by subject's clinical care team); infusion of clazakizumab 12.5 mg or 25 mg or placebo IV; collection of clinical data (vital signs, respiratory and hemodynamic support parameters)
Day 2: Day 2 laboratory studies; physical examination (as conducted by subject's clinical care team); collection of clinical data (vital signs, respiratory and hemodynamic support parameters)
Day 3: Day 3 laboratory studies; physical examination (as conducted by subject's clinical care team); assessment of eligibility for repeated clazakizumab dose; infusion of clazakizumab 12.5 mg or 25 mg or placebo IV if criteria met; collection of clinical data (vital signs, respiratory and hemodynamic support parameters)
Day 4: Day 4 laboratory studies
Day 5: Day 5 laboratory studies
Day 6: Day 6 laboratory studies
Day 7: Day 7 laboratory studies; collection of clinical data (vital signs, hemodynamic parameters, respiratory parameters)
Day 14+/−2: Day 14 laboratory studies; collection of clinical data (vital signs, hemodynamic parameters, respiratory parameters)
Day 28+/−3: Documentation of patient survival status

Additional clinical data to be collected: date of intubation (if performed); date of extubation (if performed); date of tracheostomy (if performed); date of ICU discharge (if occurs); date of hospital discharge (if occurs); date of death (if occurs)

Withdrawal/Early Termination Visit: Should the patient choose to withdraw from the study before the 30 day study duration has elapsed, the patient's choice to withdraw will be documented. The patient will be followed by the medical record to collect clinical data obtained for standard clinical care as pertains to the study outcomes, but no further study-specific blood draws will take place.

Concomitant Medications, Treatments, and Procedures: All concomitant prescription medications being administered at enrollment and during the first 28 days of study participation will be recorded. For this protocol, a prescription medication is defined as a medication that can be prescribed only by a properly authorized/licensed clinician. Medications to be reported in the CRF are concomitant prescription medications, over-the-counter medications and non-prescription medications.

### Assessment of Safety

Specification of Safety Parameters: Patients will be monitored for the manifestations of adverse events related to clazakizumab infusion. Both the nature of the event and the timing related to infusion will be considered. Given the baseline laboratory and physiologic abnormalities of the patient being enrolled in this study, assessments of clinical deterioration will be difficult to attribute with certainty to the underlying disease versus the study drug infusion. The known risks associated with clazakizumab will be considered in determining likelihood of association of AEs with the drug. We will only report adverse events which are deemed more than probably related or related to the clazakizumab infusion by the Principal Investigator.

Definition of Adverse Events (AE): An adverse event (AE) is any symptom, sign, illness or experience that develops or worsens in severity during the course of the study. Intercurrent illnesses or injuries should be regarded as adverse events. Abnormal results of diagnostic procedures are considered to be adverse events if the abnormality: results in study withdrawal; is associated with a serious adverse event; is associated with clinical signs or symptoms; leads to additional treatment or to further diagnostic tests; is considered by the investigator to be of clinical significance.

Definition of Serious Adverse Events (SAE): Adverse events are classified as serious or non-serious. A serious adverse event is any AE that is: fatal, life-threatening, requires or prolongs hospital stay, results in persistent or significant disability or incapacity, a congenital anomaly or birth defect or an important medical event. Important medical events are those that may not be immediately life threatening, but are clearly of major clinical significance. They may jeopardize the subject, and may require intervention to prevent one of the other serious outcomes noted above. For example, drug overdose or abuse, a seizure that did not result in in-patient hospitalization, or intensive treatment of bronchospasm in an emergency department would typically be considered serious. It is important to acknowledge that the patient being enrolled in this study has a high risk of mortality at the time of enrollment in the study and there is a chance that the patient may die of COVID-19 disease despite rather than because of the clazakizumab administration. In the event of mortality, every effort will be made on the part of the investigators to assess whether there was any probable association between the study drug and the patient death. In the event of patient death, the study team will consult with the clinical critical care team in an effort to determine whether the patient death was related to the underlying clinical condition (COVID-19 infection) or whether the death was unexpected given the patient's condition between the time of study drug infusion and the patient's death.

Definition of Unanticipated Problems (UP): Any incident, experience, or outcome involving a risk to subjects or others that meets all of the following criteria: 1) unexpected in nature, severity, or frequency (i.e. not described in study-related documents); 2) related or possibly related to participation in the research (i.e. possibly related means there is a reasonable possibility that the incident experience, or outcome may have been caused by the procedures involved in the research); and 3) suggests that the research places subjects or others at greater risk of harm (including physical, psychological, economic, or social harm). This definition could include an unanticipated adverse device effect, any serious adverse effect on health or safety or any life-threatening problem or death caused by, or associated with, a device, if that effect, problem, or death was not previously identified in nature, severity, or degree of incidence in the investigational plan or application (including a supplementary plan or application), or any other unanticipated serious problem associated with a device that relates to the rights, safety, or welfare of subjects (21 CFR 812.3(s)).

### Classification of an Adverse Event (AE)

Severity of AE: The following guidelines will be used to describe severity. Mild —Events require minimal or no treatment and do not interfere with the participant's daily activities. Moderate—Events result in a low level of inconvenience or concern with the therapeutic measures. Moderate events may cause some interference with functioning. Severe—Events interrupt a participant's usual daily activity and may require systemic drug therapy or other treatment. Severe events are usually potentially life-threatening or incapacitating.

Relationship of AE to Study Agent: The clinician's assessment of an AE's relationship to study agent is part of the documentation process, but it is not a factor in determining what is or is not reported in the study. If there is any doubt as to whether a clinical observation is an AE, the event should be reported. All AEs must have their relationship to study agent assessed. In a clinical trial, the study product must always be suspect. To help assess, the following guidelines are used: Related—The AE is known to occur with the study agent, there is a reasonable possibility that the study agent caused the AE, or there is a temporal relationship between the study agent and event. Reasonable possibility means that there is evidence to suggest a causal relationship between the study agent and the AE. Not Related—There is not a reasonable possibility that the administration of the study agent caused the event, there is no temporal relationship between the study agent and event onset, or an alternate etiology has been established. For all collected AEs, the clinician who examines and evaluates the participant will determine the AE's causality based on temporal relationship and his/her clinical judgment. The degree of certainty about causality will be graded using the following categories: Definitely Related—There is clear evidence to suggest a causal relationship, and other possible contributing factors can be ruled out. The clinical event, including an abnormal laboratory test result, occurs in a plausible time relationship to drug administration and cannot be explained by concurrent disease or other drugs or chemicals. The response to withdrawal of the drug (dechallenge) should be clinically plausible. The event must be pharmacologically or phenomenologically definitive, with use of a satisfactory rechallenge procedure if necessary. Probably Related—There is evidence to suggest a causal relationship, and the influence of other factors is unlikely. The clinical event, including an abnormal laboratory test result, occurs within a reasonable time after administration of the drug, is unlikely to be attributed to concurrent disease or other drugs or chemicals, and follows a clinically reasonable response on withdrawal (dechallenge).

Rechallenge information is not required to fulfill this definition. Possibly Related—There is some evidence to suggest a causal relationship (e.g., the event occurred within a reasonable time after administration of the trial medication). However, other factors may have contributed to the event (e.g., the participant's clinical condition, other concomitant events). Although an AE may rate only as “possibly related” soon after discovery, it can be flagged as requiring more information and later be upgraded to “probably related” or “definitely related,” as appropriate. Unlikely to be related—A clinical event, including an abnormal laboratory test result, whose temporal relationship to drug administration makes a causal relationship improbable (e.g., the event did not occur within a reasonable time after administration of the trial medication) and in which other drugs or chemicals or underlying disease provides plausible explanations (e.g., the participant's clinical condition, other concomitant treatments). Not Related—The AE is completely independent of study drug administration, and/or evidence exists that the event is definitely related to another etiology. There must be an alternative, definitive etiology documented by the clinician.

Expectedness: The PI will be responsible for determining whether an AE is expected or unexpected. An AE will be considered unexpected if the nature, severity, or frequency of the event is not consistent with the risk information.

Time Period and Frequency for Event Assessment and Follow-UP: The occurrence of an AE or SAE may come to the attention of study personnel during study visits and interviews of a study participant presenting for medical care, or upon review by a study monitor. All AEs including local and systemic reactions not meeting the criteria for SAEs will be captured on the appropriate CRF. Information to be collected includes event description, date and time of onset, clinician's assessment of severity, relationship to study product (assessed only by those with the training and authority to make a diagnosis), and time of resolution/stabilization of the event. All AEs occurring while on study must be documented appropriately regardless of relationship. All AEs will be followed to adequate resolution. Any medical condition that is present at the time that the participant is screened will be considered as baseline and not reported as an AE. However, if the study participant's condition deteriorates at any time during the study, it will be recorded as an AE. Unanticipated problems will be recorded in the data collection system throughout the study. Changes in the severity of an AE will be documented to allow an assessment of the duration of the event at each level of severity to be performed. AEs characterized as intermittent require documentation of onset and duration of each episode. The PI will record all reportable events with start dates occurring any time after informed consent is obtained until 7 (for non-serious AEs) or 30 days (for SAEs) after the last day of study participation. At each study visit, the investigator will inquire about the occurrence of AE/SAEs since the last visit. Events will be followed for outcome information until resolution or stabilization. All unresolved adverse events should be followed by the investigator until the events are resolved, the subject is lost to follow-up, or the adverse event is otherwise explained. At the last scheduled visit, the investigator should instruct each subject to report any subsequent event(s) that the subject, or the subject's personal physician, believes might reasonably be related to participation in this study. The investigator should notify Vitaeris of any death or adverse event occurring at any time after a subject has discontinued or terminated study participation that may reasonably be related to this study. Vitaeris should also be notified if the investigator should become aware of the development of cancer or of a congenital anomaly in a subsequently conceived offspring of a subject that has participated in this study.

Treatment Halting Rules: If a treatment related SAE occurs following the first dose, additional dosing may be halted at the discretion of the DSMB or investigator based on a benefit risk assessment. Data collection for safety would not be affected and would continue provided the patient does not elect to withdraw from the study.

### Statistical Considerations

Statistical and Analytical Plans (SAP): Descriptive statistics for the safety endpoints will be used. Regarding the secondary outcome of patient survival, too little is known about the expected clinical behavior of these patients to make meaningful projections about expected changes in mortality given drug efficacy. Below is a table summarizing power calculations for the projected sample size (N=10 in each arm) to detect the listed mortality rate change for the indicated significance levels (alpha).

| Mortality | Reduction | N = 10 per group | | |
|---|---|---|---|---|
| Rate | (%) | α = 5% | α = 10% | α = 20% |
| 5% | −90 | 0.59 | 0.68 | 0.78 |
| 10% | −80 | 0.41 | 0.52 | 0.63 |
| 15% | −70 | 0.28 | 0.37 | 0.49 |
| 20% | −60 | 0.18 | 0.26 | 0.36 |
| 25% | −50 | 0.12 | 0.18 | 0.26 |
| 30% | −40 | 0.07 | 0.12 | 0.19 |
| 35% | −30 | 0.04 | 0.08 | 0.13 |
| 40% | −20 | 0.03 | 0.05 | 0.09 |

### REFERENCES FOR EXAMPLE 6

1. Mehta P et al (2020). COVID-19: consider cytokine storm syndromes and immunosuppression. The Lancet. March 28; 395(10229):1033-1034. Published online. Available at: https://www.thelancet.com/journals/lancet/article/PIIS0140-6736(20)30628-0/fulltext
2. Karkhur S et al (2019). Interleukin-6 inhibition in the management of non-infectious uveitis and beyond. J Ophthalmic Inflamm Infect. 2019 Sep. 16; 9(1):17.
3. Weinblatt M E et al (2015). The Efficacy and Safety of Subcutaneous Clazakizumab in Patients With Moderate-To-Severe Rheumatoid Arthritis and an Inadequate Response to Methotrexate: Results From a Multinational, Phase IIb, Randomized, Double-Blind, Placebo/Active-Controlled, Dose-Ranging Study. Arthritis Rheumatol. 2015 October; 67(10):2591-600.
4. Eskandary F et al (2019). Clazakizumab in late antibody-mediated rejection: study protocol of a randomized controlled pilot trial. Trials. January 11; 20(1):37.
5. Ruan Q et al (2020) Clinical predictors of mortality due to COVID-19 based on an analysis of data of 150 patients from Wuhan, China. Intensive Care Med. 2020 Mar. 3.Epub. doi: 10.1007/s00134-020-05991-x
6. Arentz M (2020) Characteristics and Outcomes of 21 Critically Ill Patients With COVID-19 in Washington State. JAMA. Published online Mar. 19, 2020. doi:10.1001/jama.2020.4326

Schedule of Events

| | Screening | | | | | | |
|---|---|---|---|---|---|---|---|
| | (can occur on | Day | Day | Day | Day | Day | Day |
| Activity | Day 1) | 1 | 2 | 3 | 4 | 5 | 6 |
| Study team procedures | | | | | | | |
| Assess inclusion/exclusion criteria | X | | | | | | |
| Consent obtained | X | | | | | | |
| Medical History | X | | | | | | |
| Physical Exam | X | X | X | X | | | |
| Height | X | | | | | | |
| Weight | X | | | | | | |
| Vitals signs | X | X | X | X | | | |
| Document respiratory and hemodynamic support (eg vent settings, | X | X | X | X | | | |
| vasopressors, circulatory support) | | | | | | | |
| Calculation of enrollment H-score (see above table in Example 5) | X | | | | | | |
| Dose 1 administered | | X | | | | | |
| Dose 2 administered if indicated | | | | X | | | |
| Documentation of patient survival status | | | | | | | |
| Laboratory Assessments | | | | | | | |
| Comprehensive chemistry panel | X | | | | | | |
| CBC with differential | X | | | | | | |
| CRP | X | X | X | X | X | X | X |
| IL-6 | X | | | | | | |
| Total cholesterol | X | | | | | | |
| Triglycerides | X | | | | | | |
| Ferritin | X | | | | | | |
| Fibrinogen | X | | | | | | |
| D-Dimer | X | | | | | | |
| LDH | X | | | | | | |
| Troponin | X | | | | | | |
| Pregnancy Test | X | | | | | | |

| | Day | Day | Day |
|---|---|---|---|
| Activity | 7 | 14 +/− 2 | 28 +/− 2 |
| Study team procedures | | | |
| Assess inclusion/exclusion criteria | | | |
| Consent obtained | | | |
| Medical History | | | |
| Physical Exam | | | |
| Height | | | |
| Weight | | | |
| Vitals signs | X | X | |
| Document respiratory and hemodynamic | X | X | |
| support (eg vent settings, vasopressors, | | | |
| circulatory support) | | | |
| Calculation of enrollment H-score (see table | | | |
| in Example 5) | | | |
| Dose 1 administered | | | |
| Dose 2 administered if indicated | | | |
| Documentation of patient survival status | | | X |
| Laboratory Assessments | | | |
| Comprehensive chemistry panel | X | X | |
| CBC with differential | X | X | |
| CRP | X | X | |
| IL-6 | | | |
| Total cholesterol | X | X | |
| Triglycerides | X | X | |
| Ferritin | X | X | |
| Fibrinogen | X | X | |
| D-Dimer | | | |
| LDH | | | |
| Troponin | | | |
| Pregnancy Test | | | |

### Example 7

Clazakizumab for the Treatment of COVID-19 Pneumonia and Hyperinflammation

### Summary

A multi-center, double-blinded, placebo-controlled, adaptive seamless phase 2/3 trial was conducted, and enrolled patients with severe COVID-19 disease accompanied by hyperinflammation (Study No. NCT04343989). 81 patients were enrolled in the phase 2 safety and dose-finding portion of the study, and 97 patients were enrolled in the phase 3 portion. The study objective was to determine whether the direct IL-6 inhibitor clazakizumab improves survival in COVID-19 patients with hyperinflammation. In phase 2, patients were randomized 1:1:1 to receive low-dose clazakizumab (12.5 mg), high-dose clazakizumab (25 mg), or placebo. In phase 3, randomization was 1:1 between high-dose clazakizumab and placebo. The primary outcome was 28-day ventilator free survival. Secondary outcomes included overall survival and change in clinical status. Ventilator-free and overall survival were analyzed with Bayesian logistic regression. The probabilities of improved or poor outcomes, as measured by an 11-point ordinal clinical status scale, were analyzed with Bayesian cumulative odds models.

The phase 2 study demonstrated no safety concerns with clazakizumab, and suggested lack of benefit from the low-dose arm. We report on 152 patients, 74 randomized to placebo and 78 to high-dose clazakizumab. Patients who received clazakizumab had significantly greater odds of 28-day ventilator-free survival [odds ratio 3.84; P(OR>1), 99.9%] and 28-day overall survival [odds ratio 1.75; P(OR>1, 86.5%)]. At 60 days, survival was 71.8% in the clazakizumab group compared to 62.2% (p=0.28). Clazakizumab was associated with lower odds of worse clinical status at 14 and 28 days [odds ratios 0.62; P(OR<1), 94.1% and 0.58; P(OR<1), 96.3%, respectively]. For patients without acute respiratory distress syndrome (ARDS) at enrollment, 71% of clazakizumab patients improved by day 28 versus 50.8% of placebo patients (p=0.035), and only 22.6% had a poor outcome versus 44.3% of placebo patients (p=0.018). Patients with ARDS, as defined in the context of EXAMPLE 7, at enrollment demonstrated no benefit from clazakizumab in the study. In the study, clazakizumab significantly improved ventilator-free survival at 28 days in hospitalized patients with COVID-19 and hyperinflammation.

### Methods

### Design

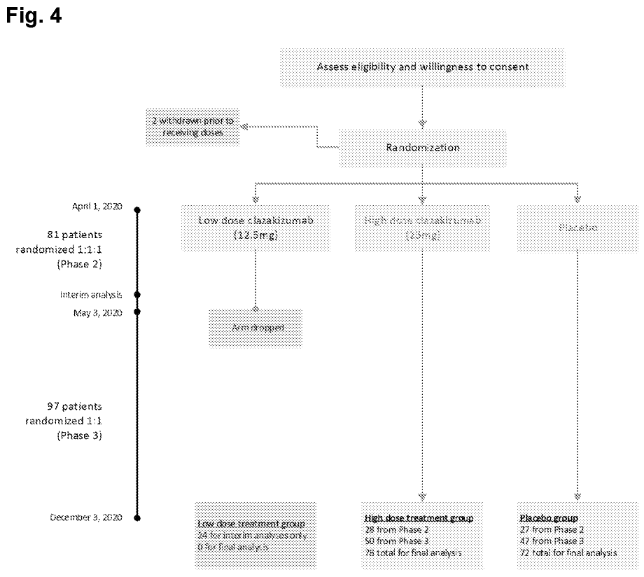

This multi-center randomized, placebo-controlled trial, enrolled patients between Apr. 1, 2020 and Dec. 3, 2020 (FIG. 4). All follow-up was completed Feb. 3, 2021. Five institutions participated: NYU Langone Health (New York, N.Y.), Columbia NewYork-Presbyterian (New York, N.Y.), the Johns Hopkins Hospital and Howard County General Hospital (Johns Hopkins Medicine, Baltimore, Md.), New York United Health Services Hospital (Binghamton, N.Y.), and the Mayo Clinic Arizona (Scottsdale, Ariz.). The phase 2 study design aimed at dose-finding and employed 1:1:1 randomization to low-dose clazakizumab (12.5 mg), high-dose clazakizumab (25 mg), and placebo. Based on an efficacy signal observed by the unblinded data safety and monitoring board (DSMB) after 81 subjects enrolled, the DSMB recommended dropping the low-dose arm. Subsequent enrollment continued in the phase 3 portion with 1:1 randomization between high-dose clazakizumab and placebo. Enrollment was completed when the target of 180 patients were randomized in both phases combined.

### Randomization and Blinding

Randomization lists were computer generated and stratified by site. Block sizes were 3 and 6 in phase 2, and 4 and 6 in phase 3. Lists generated by an unblinded statistician were distributed to a single unblinded investigator for dissemination to unblinded pharmacists. The unblinded investigator and statistician had no contact with patients and no role in data collection. Patients, treating physicians, and all other investigators remained blinded. Prepared study drug and placebo doses were indistinguishable in appearance.

### Eligibility

Eligible adult subjects had confirmed SARS-CoV-2 infection by RT-PCR testing, and respiratory manifestations including: ARDS (PaO₂/FiO₂ ratio of <200; Definition for EXAMPLE 7), hypoxemia defined by a saturation of <90% on at least 4 liters supplemental oxygen, or increasing oxygen requirements over the 24 hours immediately preceding enrollment. This criterion was included to ensure that patients suspected to be on a rapidly deteriorating trajectory could be enrolled prior to frank decompensation. Two or more indicators of hyperinflammation were required: C-reactive protein (CRP)>35 mg/L, ferritin>500 mg/ml, D-dimer>1000 ng/mL, neutrophil:lymphocyte ratio>4, lactate dehydrogenase (LDH)>200U/L, or elevated troponin in the absence of cardiac disease. IL-6 levels were drawn prior to dosing of study drug but were not part of the enrollment criteria due to the test turnaround time. Subjects with capacity provided written consent; consent was otherwise obtained from legally authorized representatives.

Patients were excluded in the event of an irreversible condition deemed non-survivable, active inflammatory bowel disease, active untreated diverticulitis, untreated bacteremia, pregnancy, or known hypersensitivity to clazakizumab.

Subjects were permitted to receive all available therapies, excluding other IL-6 pathway inhibitors.

### Oversight

This was an investigator-initiated trial designed by the NYU Langone research team. The protocol was approved by the Institutional Review Boards (IRB) at each participating site. Each site's lead investigator submitted an investigational new drug (IND) application to the Food and Drug Administration (FDA) to use clazakizumab, and each IND was approved prior to site activation. A single DSMB reviewed all participating site data bi-weekly for the first two months, and monthly thereafter.

### Study Procedures

Baseline laboratory tests to assess eligibility were performed at screening. Consented patients were randomized, and the first dose of study drug was administered on the day of consent or the following day. Study day 1 was defined as the day of first study drug administration. Clazakizumab or placebo doses were administered intravenously over 30 minutes. Daily serum CRP levels were drawn on days 1-7 and 14. On day 3, the CRP was compared to day 1; if the CRP failed to decrease by at least 50%, a second dose identical to the day 1 dose (clazakizumab or placebo) was administered on day 3. Clinical parameters including vital signs, respiratory status, concomitant medications, and adverse events (AEs) were collected during the inpatient hospitalization up to 28 days. Patients were followed remotely post-discharge for clinical status and AEs to 60 days. AEs were considered serious (SAEs) if the outcome was death, or if they were otherwise unexpected for critically-ill patients with COVID-19. The World Health Organization (WHO) ordinal 11-point scale, delineated in the Table 1 footnote,¹⁴ was used to capture clinical status at baseline, and days 14, 28 and 60. For patients discharged from the hospital prior to day 14, the WHO score on the day of discharge was captured. Outpatient day 28 and day 60 visits were conducted by phone.

### Outcome Measures

The primary outcome was ventilator-free survival at 28 days. Secondary outcomes included overall patient survival at 28 and 60 days, clinical outcome based on WHO scores, and incidence of AEs. Subgroup analyses based on the presence of absence of ARDS at enrollment were performed.

### Statistical Analysis

The trial was initially designed as a randomized phase 2 dose-finding study, with 20 patients in each of three arms to provide at least 80% power to detect a 70-90% reduction in the 28-day mortality rate, assuming mortality in the control group of 5-15%. With rapid patient accrual in April 2020 and the urgency of the pandemic surge in New York City, we amended the protocol as an adaptive seamless phase 2/3 design, enabling efficient transition to a phase 3 study of the more effective dose identified in phase 2. This amendment was approved by the IRB, DSMB and the FDA. The phase 3 portion of the trial enrolled subjects randomized 1:1 in order to yield a target of 150 total subjects in the clazakizumab and placebo groups, and provided approximately 80% power to detect a 40-90% reduction in the 28-day mortality rate, assuming mortality in the control group of 5-30%.

In the phase 2 portion, the DSMB performed interim analyses for efficacy and futility after every 30 patients had outcome information, following the approach of Stallard¹⁵ to determine which arm(s) should proceed to the phase 3 portion. Assuming standardized effect sizes of 0.3 and 0.6 for the low- and high-dose clazakizumab arms, respectively, the design ensured more than 95% probability of selecting the higher-performing arm to continue from phase 2 to phase 3. In addition, the design afforded more than 85% power to detect a meaningful improvement in at least one active arm at the phase 3 analysis.

Baseline characteristics were compared between arms to assess balance. AEs were summarized, and those deemed potentially related to clazakizumab were assessed separately. Analysis of the primary outcome was based on Bayesian logistic regression. The same approach was used for overall survival. Here, an odds ratio (OR)>1 indicates a beneficial effect of clazakizumab. The ordinal WHO scale was analyzed using Bayesian cumulative proportional odds regression, modeling the cumulative probability for the WHO clinical status to be at a given level between 0 and 10 (0: uninfected, no viral RNA detected, 10: dead) or higher (i.e., worse). The same approach was used for WHO scores at day 14 and day 28. Here, OR<1 indicates a beneficial effect of clazakizumab. The Bayesian logistic and cumulative odds models were adjusted for age, sex, baseline WHO scores, and site. For each of these analyses, we estimated the posterior distribution of the OR and its 95% credible interval. Each set of analyses was conducted using both a skeptical prior and a noninformative prior. We report the results from the analysis that used the noninformative prior for the OR (mean=0, variance=10²).

We analyzed two additional outcomes sometimes used in COVID-19 treatment research: (i) poor outcome as defined by a WHO score between 6 and 10, and (ii) improved outcome defined by a 2-point or greater decrease on the WHO scale. Frequentist analysis based on chi-square test was employed. Additionally, to provide reference to reports presenting frequentist statistical analysis and associated p-values, the primary outcome was compared between arms using a chi-squared test.

### Results

### Patients

Across five sites, 180 patients underwent randomization. In the phase 2 dose-finding portion, 81 patients were randomized 1:1:1 (26 to low-dose clazakizumab, 28 to high-dose clazakizumab, and 27 to placebo; FIG. 4). Interim analysis indicated lack of benefit of low-dose clazakizumab, and this arm was dropped. No low-dose patient data are included in the following analyses and all references therein to the “clazakizumab group” indicate patients who received high-dose. In the phase 3 portion, 99 patients were randomized 1:1 to receive either high-dose clazakizumab or placebo. Two patients were withdrawn after consent due to rapid changes in clinical status; neither withdrawn patient received study drug nor was included in data analysis. This left 97 patients who proceeded to dosing (50 with high-dose clazakizumab and 47 with placebo). The groups for final analysis, combining the patients in both phases, totaled 78 randomized to high-dose clazakizumab and 74 randomized to placebo. The demographic, medical history, and baseline clinical characteristics were similar between the clazakizumab and placebo groups (Table 1). The mean age was 61.8 years and 70.4% were male. Overall, 34.2% were White, 18.4% were Black, 10.5% were Asian; 27% were of Hispanic ethnicity. Hypertension (63.2%), diabetes (42.1%), and cardiac disease (34.2%) were the most common pre-existing conditions. The median number of days from symptom onset to first dose of study drug was 10 (interquartile range [IQR] 7-13) and from positive test to first dose was 4 (IQR 2-7). Corticosteroids and remdesivir were concurrently administered in 75% and 49.3%, respectively, and this was similar among those who received clazakizumab and placebo. All patients had a baseline WHO score between 5 and 9.59. 2% required non-invasive ventilation or high-flow oxygen delivery, and 24.3% were intubated at the time of enrollment. The median baseline inflammatory marker levels were: IL-6 26 pg/ml (IQR 10-98.6), CRP 155.5 mg/L (IQR 90.6-240.6), ferritin 1166 ng/mL (IQR 693.5-2011), D-dimer 914 ng/mL (IQR 542.5-2713.8), fibrinogen 645 mg/dL (IQR 536.5-700), LDH 515U/L (IQR 410-680.5), troponin 0.04 ng/mL (IQR 0.01-0.1), and neutrophil:lymphocyte ratio 9.4 (IQR 5.8-16.4).

### Change in C-Reactive Protein and Repeated Dosing

Clazakizumab was associated with a significant decrease in CRP compared to placebo. In the clazakizumab group, median CRP decreased from baseline 161.9 mg/L (IQR 92.2-239.1) to 60.8 mg/L (IQR 32.0-120.0) on day 3 whereas in the placebo group, median CRP decreased from baseline 153.3 mg/L (IQR 86.9-241.6) to 113.2 mg/L (IQR 56.6-228.1) on day 3 (p<0.001). Based on the change in CRP values, 70.3% of placebo patients received a repeated dose of normal saline on day 3, while only 38.5% of patients who received clazakizumab received a second dose on day 3 (p<0.001).

### Primary and Key Secondary Outcomes

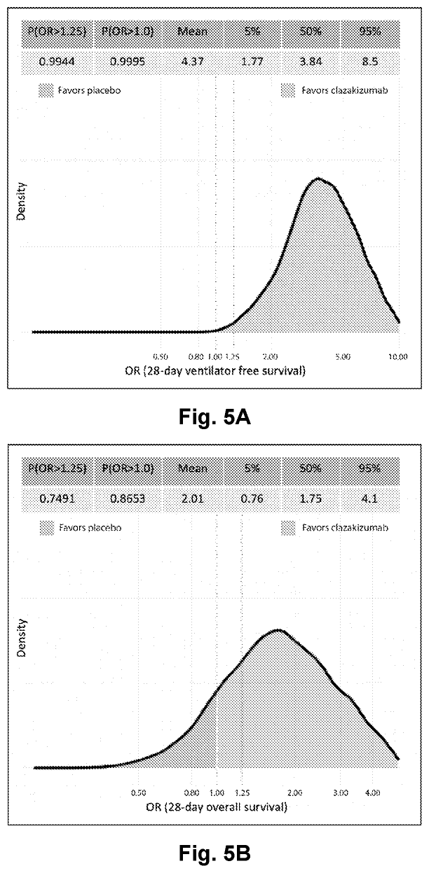

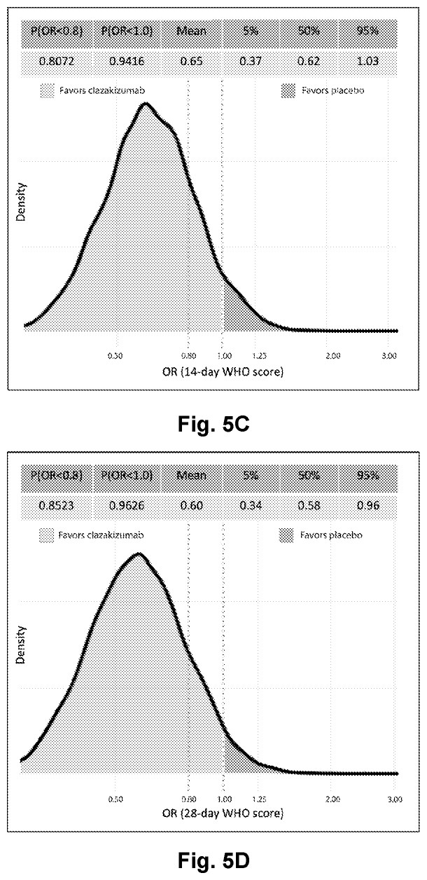

A total of 96 patients (63.2%) were alive and ventilator free at 28 days. In the clazakizumab group, 55 (70.5%) achieved this outcome compared to 41 (55.4%) in the placebo group; the chi-squared test comparing the two groups resulted in a p-value of 0.08. Bayesian logistic regression models adjusted for age, sex, baseline WHO score, and site, indicated that patients in the clazakizumab group had a significantly greater odds of 28-day ventilator free survival than those receiving placebo (FIG. 5A). For this primary outcome, the estimated median of the posterior distribution of the OR comparing the clazakizumab and placebo groups was 3.84 [P(OR>1), 99.9%]. A total of 113 patients were alive at 28 days. In the clazakizumab group, 59 (75.6%) were alive at 28 days, compared to 54 (73.0%) in the placebo group; the chi-squared test comparing the groups resulted in a p-value of 0.85. Based on a Bayesian model for overall 28-day survival, adjusting for baseline covariates (FIG. 5B), the estimated median of the posterior distribution of the OR comparing the clazakizumab and placebo groups was 1.75 [P(OR>1), 86.5%].

We also measured treatment effect by capturing clinical status as measured by the WHO scores at 14 and 28 days post-treatment. Using Bayesian cumulative odds models, the median predicted OR of a higher WHO score (i.e., worse clinical outcome) for the clazakizumab group was 0.62 [P(OR<1), 94.2%; FIG. 2C] at 14 days and 0.58 at 28 days [P(OR<1), 96.3%; FIG. 5D].

We further assessed 28-day clinical change by subgroup based on whether ARDS was present or absent at the time of enrollment (Table 2). We defined a clinically improved outcome as a decrease in WHO score by at least 2 points between enrollment and day 28. A total of 123 patients did not meet criteria for ARDS at baseline. 72% (44/62) of patients without ARDS who received clazakizumab demonstrated an improved outcome at 28-days. This was significantly greater than the 50.8% (31/61) of patients without ARDS who received placebo (p=0.035). We defined a clinically poor outcome as a WHO score at day 28 of 6-10. 22.6% (14/62) of patients without ARDS had a poor outcome at 28-days. This was significantly less than the 44.3% (27/61) of patients 44 (71.0%) of patients without ARDS who received placebo (p=0.018).

For patients with ARDS at enrollment, clazakizumab was neither associated with a greater proportion of improved outcomes (37.5% of clazakizumab patients improved versus 46.2% of placebo patients, p=0.927), nor a smaller proportion of poor outcomes (68.8% of clazakizumab patients had poor outcome versus 69.2% placebo patients, p=1).

At 60 days, 56 of the clazakizumab patients (71.8%) survived compared to 46 placebo patients (62.2%). While the odds of survival were 55% greater in the clazakizumab patients, this difference was not statistically significant (OR 1.55, 95% Cl 0.78-3.06; p=0.275). We also compared the number of days of ventilator dependence in the surviving patients within the 60-day study period. The clazakizumab patients were ventilator-dependent for an average of 4.73±12.1 days compared to 9.26±17.7 days for placebo patients; this difference also failed to achieve statistical significance (p=0.255).

### Safety Outcomes

COVID-19 expected adverse events including infections, thromboembolic events, and acute kidney injury, were observed at similar frequency between patients who received clazakizumab and placebo (Table 3). Adverse events of specific concern with clazakizumab include hypersensitivity-type reactions, transaminitis, elevation in serum lipids, and bowel perforations. No bowel perforations or hypersensitivity reactions were observed in any patient. Elevation in transaminases and lipids occurred at similar rates in the clazakizumab and placebo groups.

### Discussion

We conducted a multi-center seamless phase 2/3 randomized controlled trial of clazakizumab for the treatment of patients with severe COVID-19 disease and hyperinflammation. We observed that high-dose (25 mg) clazakizumab improved 28-day ventilator free survival, as well as overall 28-day survival, compared to placebo. The estimated posterior median of the odds ratio for 28-day ventilator free survival was 3.84, with a 95% Cl of 1.77-8.5, which constitutes very strong evidence of clinically meaningful improvement. In addition to this primary outcome, evaluation of clinical disease progression illustrates that compared to placebo, patients given clazakizumab were more likely to have had a 2-point or greater improvement in WHO score over 28 days. Similarly, 28-day scores were more likely to be unfavorable (scores of 6-10) among those who received placebo compared to clazakizumab. These 28-day statistical analyses showed a striking degree of consistency of both strength and direction of effect in favor of clazakizumab. There was a trend toward overall improved 60-day survival in the clazakizumab group, as well as a shorter duration of ventilator dependence, but these trends were not statistically significant. The high mortality amongst patients with ARDS at enrollment reduced the sample size of the populated that appeared to benefit, which limited the power of some analyses.

The dose-finding phase 2 portion provided early suggestion of benefit from high-dose clazakizumab without indication of safety concerns, and suggested that there was likely no benefit to low-dose clazakizumab, leading to this arm being dropped after interim analyses. Throughout the study, AEs including infections did not occur at a higher rate in patients receiving clazakizumab compared to placebo. No hypersensitivity reactions to clazakizumab occurred.

The data not only reveal that clazakizumab appears to benefit patients with severe respiratory manifestations of COVID-19 and hyperinflammation, but also indicate what is likely to be the optimal timing of administration. Subgroup analyses suggest that clazakizumab did not benefit patients whose clinical status was dire at the time of enrollment, namely those who had already developed ARDS as defined in the context of EXAMPLE 7. This indicates that clazakizumab should not be withheld until all other therapies are exhausted and patients have reached end-stage respiratory failure.

Rather, it should be administered no later than the point when patients begin to transition from moderate to severe disease. Those who rapidly develop new or escalating oxygen requirements, or those just initiating non-invasive ventilation, appear to benefit most.

In only a year since its discovery, the novel coronavirus SARS-CoV-2 has exacted an enormous global toll. Optimism rightly spurred by the development of efficacious vaccines¹⁶⁻¹⁸ remains tempered by the reality of vaccine administration pace, as well as the emergence of variant viral strains.¹⁹ In the meantime, effective treatments are needed to abrogate morbidity and mortality. Remdesivir and corticosteroids appear to provide modest but real benefit.²⁰⁻²² Patients with critical illness have been theorized to benefit from cytokine inhibitory therapies,⁹ but subsequent open-label and randomized controlled studies of IL-6R inhibitors have shown mixed results.^{11,23-26} Compared to IL-6R antagonists, the mechanism of action of clazakizumab as a direct IL-6 ligand inhibitor is potentially advantageous. IL-6R is upregulated in response to influenza infection,²⁷ and if similar upregulation occurs in SARS-CoV-2 infection, IL-6R inhibitor drugs may be rapidly bound and sequestered, potentially limiting their efficacy.²⁸⁻³⁰ This double-blinded, randomized, placebo-controlled trial provides evidence that the direct IL-6 inhibitor clazakizumab may be lifesaving if administered to COVID-19 patients at the outset of disease progression marked by hyperinflammation.

### REFERENCES FOR EXAMPLE 7

1. Johns Hopkins Coronavirus Resource Center. https://coronavirus.jhu.edu/map.html. Accessed Mar. 7, 2021.
2. Huang C, Wang Y, Li X, et al. Clinical features of patients infected with 2019 novel coronavirus in Wuhan, China. Lancet. 2020; 395(10223):497-506.
3. Zhou F, Yu T, Du R, et al. Clinical course and risk factors for mortality of adult inpatients with COVID-19 in Wuhan, China: a retrospective cohort study. Lancet. 2020; 395(10229):1054-1062.
4. Gustine J N, Jones D. Immunopathology of Hyperinflammation in COVID-19. Am J Pathol. 2020.
5. Rubio-Rivas M, Ronda M, Padulles A, et al. Beneficial effect of corticosteroids in preventing mortality in patients receiving tocilizumab to treat severe COVID-19 illness. Int J Infect Dis. 2020; 101:290-297.
6. Zheng K L, Xu Y, Guo Y F, et al. Efficacy and safety of tocilizumab in COVID-19 patients. Aging (Albany N.Y.). 2020; 12(19):18878-18888.
7. Petrak R M, Skorodin N C, Van Hise N W, et al. Tocilizumab as a Therapeutic Agent for Critically Ill Patients Infected with SARS-CoV-2. Clin Trans/Sci. 2020.
8. Price C C, Altice F L, Shyr Y, et al. Tocilizumab Treatment for Cytokine Release Syndrome in Hospitalized Patients With Coronavirus Disease 2019: Survival and Clinical Outcomes. Chest. 2020; 158(4):1397-1408.
9. Lewis T C, Adhikari S, Tatapudi V, et al. A Propensity-Matched Cohort Study of Tocilizumab in Patients With Coronavirus Disease 2019. Crit Care Explor. 2020; 2(11):e0283.
10. Kaye A G, Siegel R. The efficacy of IL-6 inhibitor Tocilizumab in reducing severe COVID-19 mortality: a systematic review. Peer J. 2020; 8:e10322.
11. Rosas 10, Brau N, Waters M, et al. Tocilizumab in Hospitalized Patients with Severe Covid-19 Pneumonia. N Engl J Med. 2021.
12. Weinblatt M E, Mease P, Mysler E, et al. The efficacy and safety of subcutaneous clazakizumab in patients with moderate-to-severe rheumatoid arthritis and an inadequate response to methotrexate: results from a multinational, phase IIb, randomized, double-blind, placebo/active-controlled, dose-ranging study. Arthritis Rheumatol. 2015; 67(10):2591-2600.
13. Eskandary F, Durr M, Budde K, et al. Clazakizumab in late antibody-mediated rejection: study protocol of a randomized controlled pilot trial. Trials. 2019; 20(1):37.
14. Characterisation WHOWGotC, Management of C-i. A minimal common outcome measure set for COVID-19 clinical research. Lancet Infect Dis. 2020; 20(8):e192-e197.
15. Stallard N. A confirmatory seamless phase II/III clinical trial design incorporating short-term endpoint information. Stat Med. 2010; 29(9):959-971.
16. Baden L R, El Sahly H M, Essink B, et al. Efficacy and Safety of the mRNA-1273 SARS-CoV-2 Vaccine. N Engl J Med. 2020.
17. Polack F P, Thomas S J, Kitchin N, et al. Safety and Efficacy of the BNT162b2 mRNA Covid-19 Vaccine. N Engl J Med. 2020; 383(27):2603-2615.
18. Voysey M, Clemens S A C, Madhi S A, et al. Safety and efficacy of the ChAdOx1 nCoV-19 vaccine (AZD1222) against SARS-CoV-2: an interim analysis of four randomised controlled trials in Brazil, South Africa, and the U K. Lancet. 2021; 397(10269):99-111.
19. Korber B, Fischer W M, Gnanakaran S, et al. Tracking Changes in SARS-CoV-2 Spike: Evidence that D614G Increases Infectivity of the COVID-19 Virus. Cell. 2020; 182(4):812-827 e819.
20. Beigel J H, Tomashek K M, Dodd L E, et al. Remdesivir for the Treatment of Covid-19—Final Report. N Engl J Med. 2020; 383(19):1813-1826.
21. Group R C, Horby P, Lim W S, et al. Dexamethasone in Hospitalized Patients with Covid-19—Preliminary Report. N Engl J Med. 2020.
22. Group WHOREAfC-T W, Sterne J A C, Murthy S, et al. Association Between Administration of Systemic Corticosteroids and Mortality Among Critically Ill Patients With COVID-19: A Meta-analysis. JAMA. 2020; 324(13):1330-1341.
23. Salama C, Han J, Yau L, et al. Tocilizumab in Patients Hospitalized with Covid-19 Pneumonia. N Engl J Med. 2021; 384(1):20-30.
24. Leon Lopez R, Fernandez S C, Limia Perez L, et al. Efficacy and safety of early treatment with sarilumab in hospitalised adults with COVID-19 presenting cytokine release syndrome (SARICOR STUDY): protocol of a phase II, open-label, randomised, multicentre, controlled clinical trial. BMJ Open. 2020; 10(11):e039951.
25. Garcia-Vicuna R, Abad-Santos F, Gonzalez-Alvaro I, Ramos-Lima F, Sanz J S. Subcutaneous Sarilumab in hospitalised patients with moderate-severe COVID-19 infection compared to the standard of care (SARCOVID): a structured summary of a study protocol for a randomised controlled trial. Trials. 2020; 21(1):772.
26. Caballero Bermejo A F, Ruiz-Antoran B, Fernandez Cruz A, et al. Sarilumab versus standard of care for the early treatment of COVID-19 pneumonia in hospitalized patients: SARTRE: a structured summary of a study protocol for a randomised controlled trial. Trials. 2020; 21(1):794.
27. Wang J, Wang Q, Han T, et al. Soluble interleukin-6 receptor is elevated during influenza A virus infection and mediates the IL-6 and IL-32 inflammatory cytokine burst. Cell Mol Immunol. 2015; 12(5):633-644.
28. Stone J H, Frigault M J, Serling-Boyd N J, et al. Efficacy of Tocilizumab in Patients Hospitalized with Covid-19. N Engl J Med. 2020; 383(24):2333-2344.
29. Hermine O, Mariette X, Tharaux P L, et al. Effect of Tocilizumab vs Usual Care in Adults Hospitalized With COVID-19 and Moderate or Severe Pneumonia: A Randomized Clinical Trial. JAMA Intern Med. 2021; 181(1):32-40.
30. Della-Torre E, Campochiaro C, Cavalli G, et al. Interleukin-6 blockade with sarilumab in severe COVID-19 pneumonia with systemic hyperinflammation: an open-label cohort study. Ann Rheum Dis. 2020; 79(10):1277-1285.

TABLE 1
Demographic and Baseline Clinical Characteristics

| | All | Clazakizumab | Placebo |
|---|---|---|---|
| Characteristic | (N = 152) | (N = 78) | (N = 74) |
| Age—yr ± SD | 61.8 ± 12.2 | 63.7 ± 11.1 | 59.8 ± 13.1 |
| Male sex—no. (%) | 107 (70.4) | 53 (67.9) | 54 (73.0) |
| Race or ethnic group*—no. (%) | | | |
| African American or Black | 28 (18.4) | 16 (20.5) | 12 (16.2) |
| Asian | 16 (10.5) | 7 (9.0) | 9 (12.2) |
| White | 52 (34.2) | 25 (32.1) | 27 (36.5) |
| Hispanic or Latino | 41 (27.0) | 17 (21.8) | 24 (32.4) |
| Other/mixed ancestry | 27 (17.8) | 12 (15.4) | 15 (20.3) |
| Not reported | 29 (19.1) | 18 (23.1) | 11 (14.9) |
| Body mass index—median (IQR) | 28.4 (24.6 to 32.2) | 29.0 (25.1 to 32.9) | 28.2 (24.2 to 32.2) |
| Median time (IQR) from symptom onset | 10.0 (7 to 13) | 10.0 (6 to 13) | 10.0 (7 to 13) |
| to first dose—days | | | |
| Median time (IQR) from positive | 4.00 (2 to 7) | 3.00 (2 to 6) | 4.50 (2 to 8) |
| COVID-19 test to first dose—days | | | |
| Intubated at screening—no. (%) | 37 (24.3) | 20 (25.6) | 17 (23.0) |
| Coexisting conditions—no. (%) | | | |
| Hypertension | 96 (63.2) | 51 (65.4) | 45 (60.8) |
| Severe obesity (BMI > 40) | 17 (11.2) | 7 (9.0) | 10 (13.5) |
| Type 2 diabetes | 64 (42.1) | 37 (47.3) | 27 (36.5) |
| Cardiac disease other than HTN | 52 (34.2) | 32 (41.0) | 20 (27.0) |
| Tobacco use | 11 (7.2) | 6 (7.7) | 5 (6.8) |
| Underlying pulmonary disease | 25 (16.4) | 13 (16.7) | 12 (16.2) |
| Chronic immunosuppression | 11 (7.2) | 7 (9.0) | 4 (5.4) |
| History of malignancy (other than | 13 (8.6) | 10 (12.8) | 3 (4.1) |
| skin cancer) | | | |
| Received corticosteroids—no. (%) | 114 (75.0) | 59 (75.6) | 55 (74.3) |
| Received remdesivir—no. (%) | 75 (49.3) | 36 (46.2) | 39 (52.7) |
| Baseline WHO score^{§}—no. (%) | | | |
| Level 5 | 25 (16.4) | 13 (16.7) | 12 (16.2) |
| Level 6 | 90 (59.2) | 45 (57.7) | 45 (60.8) |
| Level 7 | 8 (5.3) | 4 (5.1) | 4 (5.4) |
| Level 8 | 20 (13.2) | 13 (16.7) | 7 (9.5) |
| Level 9 | 9 (5.9) | 3 (3.8) | 6 (8.1) |
| Baseline inflammatory | | | |
| markers^{¥}—median (IQR) | | | |
| IL-6 level—pg/mL | 26 (10 to 98.6) | 37.6 (14.8 to 120) | 20 (8.1 to 53.9) |
| C-reactive protein—mg/L | 155.5 (90.6 to 240.6) | 160.9 (92.2 to 239.1) | 153.3 (86.9 to 241.6) |
| Ferritin—ng/mL | 1166 (693.5 to 2011) | 1430 (732 to 2372) | 1038.5 (679.5 to 1593) |
| D-dimer—ng/mL | 914 (542.5 to 2713.8) | 1255 (588.3 to 427.3) | 813.5 (531.3 to 1861) |
| Fibrinogen—(mg/dL) | 645 (536.5 to 700) | 673.5 (539.5 to 700) | 630 (524 to 700) |
| Lactate dehydrogenase—U/L | 515 (410 to 680.5) | 517 (406.3 to 700) | 508 (415.5 to 663.5) |
| Troponin—ng/mL | 0.04 (0.01 to 0.1) | 0.04 (0.01 to 0.1) | 0.04 (0.01 to 0.1) |
| Neutrophil:Lymphocyte ratio | 9.4 (5.8 to 16.4) | 8.8 (5.9 to 15.1) | 11 (5.4 to 17.8) |

*Race and ethnic group self-reported by patients
^{‡}ARDS, acute respiratory distress syndrome (PaO₂/FiO₂ ratio <200) (Definition for EXAMPLE 7)
^{§}WHO, World Health Organization, ordinal scale for clinical outcomes scores are as follows:
0, uninfected, no viral RNA detected;
1, asymptomatic; viral RNA detected;
2, symptomatic, independent;
3, symptomatic, assistance needed;
4, hospitalized, no oxygen therapy;
5, hospitalized, oxygen by simple mask or nasal prongs;
6, hospitalized, oxygen by non-invasive ventilation or high flow nasal cannula;
7, intubation and mechanical ventilation, PaO₂/FiO₂ ≥150 or SpO₂/FiO₂ ≥200;
8, mechanical ventilation, PaO₂/FiO₂ <150 or SpO₂/FiO₂ <200 or vasopressors;
9, mechanical ventilation, PaO₂/FiO₂ <200 and vasopressors, dialysis or ECMO
^{¥}Inclusion criteria required two or more parameters (exclusive of IL-6) to be elevated as indicated in Methods. As such, not all patients had this full inflammatory panel tested. This panel was completed as such: CRP, 100% of subjects; IL-6, 94.1%; ferritin, 99%; D-dimer, 100%; fibrinogen, 86.5%; LDH, 94.1%; troponin, 98%; neutrophil:lymphocyte ratio, 96%

TABLE 2
Clinical Outcomes at 28 Days

| Clinical status at enrollment | | |
|---|---|---|
| ARDS absent | ARDS present | All |

A. Assessment of improvement*

| | | | | |
|---|---|---|---|---|
| Clazakizumab | No. in group | 62 | 16 | 78 |
| | No. improved | 44 (71.0) | 6 (37.5) | 50 (64.1) |
| | (%) | | | |
| Placebo | No. in group | 61 | 13 | 74 |
| | No. improved | 31 (50.8) | 6 (46.2) | 37 (50) |
| | (%) | | | |
| | p-value | 0.035 | 0.927 | 0.111 |

B. Assessment of poor outcome^{‡}

| | | | | |
|---|---|---|---|---|
| Clazakizumab | No. in group | 62 | 16 | 78 |
| | No. scoring | 14 (22.6) | 11 (68.8) | 25 (32.1) |
| | 6-10 (%) | | | |
| Placebo | No. in group | 61 | 13 | 74 |
| | No. scoring | 27 (44.3) | 9 (69.2) | 36 (48.6) |
| | 6-10 (%) | | | |
| | p-value | 0.018 | 1 | 0.055 |

*Improvement defined as WHO clinical score having decreased by two or more points at 28 days
^{‡}Poor outcome defined as having WHO clinical score of 6-10 at 28 days
p-values determined by chi-square test

TABLE 3
Adverse Events (AE) Summary

| | | |
|---|---|---|
| All | Clazakizumab | Placebo |
| (N = 152) | (N = 78) | (N = 74) |

Covid-19 expected AEs

| | | | |
|---|---|---|---|
| Acute kidney injury—no. (%) | 34(22.4) | 15(19.2) | 19(25.7) |
| Infection*—no. (%) | 35(23.0) | 14(17.9) | 21(28.4) |
| Thromboembolic event—no. (%) | 8(5.3) | 3(3.8) | 5(6.8) |

Summary of Covid-19 expected AEs

| | | | |
|---|---|---|---|
| Patients with Covid-19 expected AEs—no. (%) | 55(36.2) | 21(26.9) | 34(45.9) |
| Total AEs—no. | 77 | 32 | 45 |
| Total patient-days—no. | 3991 | 1628 | 2363 |
| AE rate (AEs/patient-days) | 0.0193 | 0.0197 | 0.019 |

Clazakizumab expected AEs

| | | | |
|---|---|---|---|
| Hypersensitivity-type reaction—no. (%) | 0(0) | 0(0) | 0(0) |
| LFTs > 5 × ULN^{‡}—no. (%) | 13(8.6) | 5(6.4) | 8(10.8) |
| Hyperlipidemia—no. (%) | 6(3.9) | 2(2.6) | 4(5.4) |
| Bowel perforation—no. (%) | 0(0) | 0(0) | 0(0) |

Summary of clazakizumab expected AEs

| | | | |
|---|---|---|---|
| Patients with clazakizumab expected AEs—no. (%) | 18(11.8) | 7(9.0) | 11(14.9) |
| Total AEs—no. | 19 | 7 | 12 |
| Total patient-days—no. | 3991 | 1628 | 2363 |
| AE rate (AEs/patient-days) | 0.0048 | 0.0043 | 0.0051 |

*Includes any diagnosed infection other than Covid-19
^{‡}LFTs, liver function tests, greater than 5-times upper limit of normal

1. A method of treating acute or chronic respiratory distress syndrome (ARDS or CRDS) or reducing the risk of ARDS in a human subject who has or is suspected of having a coronavirus infection, comprising administering to said subject an effective amount of an anti-human interleukin-6 (IL-6) antibody.

2. The method of claim 1, wherein the coronavirus infection is COVID-19.

3. A method of treating a human subject who has or is suspected of having a coronavirus infection, comprising administering to said subject an effective amount of an anti-human interleukin-6 (IL-6) antibody, optionally wherein the subject has mild ARDS or does not have acute respiratory distress syndrome (ARDS).

4.-6. (canceled)

7. The method of claim 1, wherein the subject has a COVID-19 infection.

8.-10. (canceled)

11. The method of claim 7, wherein the subject has a COVID-19 WHO score of 7 or less.

12. The method of claim 7, wherein the subject has a COVID-19 WHO score of 6 or less, and/or wherein the subject has not been intubated.

13. The method of claim 1, wherein the subject has or is suspected of having cytokine storm syndrome.

14. (canceled)

15. The method of claim 1, wherein the anti-IL-6 antibody is administered at a dose ranging from 10-25 mg.

16. The method of claim 15, wherein the anti-IL-6 antibody is administered at a dose of 10 mg, 12.5 mg, or 25 mg.

17. (canceled)

18. The method of claim 1, wherein the anti-IL-6 antibody is administered to the subject only once or at least twice to the subject with at least a 48-hour interval between doses.

19. (canceled)

20. The method of claim 1, wherein a 10 mg or 12.5 mg or 25 mg dose of the anti-IL-6 antibody is administered every 2 days, every 3 days, twice weekly, every 1 week, every 2 weeks, every 4 weeks or monthly.

21. (canceled)

22. The method of claim 1, wherein:
a) the anti-IL-6 antibody inhibits the binding of human IL-6 to human gp130 and/or to human IL-6R1;
b) the antibody comprises a light chain comprising a variable light chain polypeptide comprising light chain complementarity defining region (CDRs) comprising amino acid sequences of SEQ ID NOs: 4, 5 and 6, and a heavy chain comprising heavy chain CDRs comprising amino acid sequences of SEQ ID NOs: 7, 8 or 120, and 9;
c) the anti-human IL-6 antibody comprises a heavy chain comprising the amino acid sequence of SEQ ID NO: 704 or 745 and comprises a light chain comprising the amino acid sequence of SEQ ID NO: 702 or 746;
d) the anti-TL-6 antibody is a humanized, single chain, or chimeric antibody, or an antibody fragment; and/or
e) the anti-TL-6 antibody comprises a human constant region;
f) the anti-IL-6 antibody comprises a heavy chain comprising the amino acid sequence of SEQ ID NO: 657 and a light chain comprising the amino acid sequence of SEQ ID NO: 709; and/or
g) the anti-IL-6 antibody is clazakizumab.

23.-27. (canceled)

28. The method of claim 22, wherein the anti-TL-6 antibody comprises a human IgG1 constant region.

29. (canceled)

30. The method of claim 28, wherein the anti-TL-6 antibody comprises a human IgG1 light chain constant region comprising the amino acid sequence of SEQ ID NO: 586 and a human IgG1 heavy chain constant region comprising the amino acid sequence of SEQ ID NO: 588.

31. (canceled)

32. (canceled)

33. A method of treating ARDS or CRDS or cytokine storm syndrome in a human subject who has or is suspected of having a viral, fungal, or bacterial infection, comprising administering to said subject at least one dose of 10 mg, 12.5 mg, or 25 mg of an anti-human interleukin-6 (IL-6) antibody, optionally wherein the subject is receiving supplemental oxygen or is on a mechanical ventilator or respirator prior to treatment, wherein the anti-IL-6 antibody:
a. comprises a variable light chain polypeptide comprising light chain complementarity defining region (CDRs) comprising amino acid sequences of SEQ ID NOs: 4, 5 and 6, and a heavy chain comprising heavy chain CDRs comprising amino acid sequences of SEQ ID NOs: 7, 8 or 120, and 9;
b. comprises a heavy chain comprising the amino acid sequence of SEQ ID NO: 704 or 745 and comprises a light chain comprising the amino acid sequence of SEQ ID NO: 702 or 746; or
c. is clazakizumab.

34. (canceled)

35. The method of claim 33, wherein (a) the subject does not have ARDS prior to treatment and wherein the treatment reduces the risk of the subject developing ARDS, or (b) the subject has mild ARDS prior to treatment and wherein the treatment reduces the risk of the subject developing moderate or severe ARDS.

36. (canceled)

37. The method of claim 33, wherein the subject has or is suspected of having a COVID-19 infection.

38. The method of claim 33, wherein the subject has pneumonia.

39. (canceled)

40. (canceled)

41. The method of claim 37, wherein the subject has a COVID-19 WHO score of 7 or less.

42. The method of claim 37, wherein the subject has a COVID-19 WHO score of 6 or less, and/or wherein the subject has not been intubated.

43. The method of claim 33, wherein the anti-TL-6 antibody is administered only once or at least twice to the subject with at least a 48-hour interval between doses.

44. (canceled)

45. The method of claim 33, wherein a 10 mg, 12.5 mg, or 25 mg dose of the anti-IL-6 antibody is administered every 2 days, every 3 days, twice weekly, every 1 week, every 2 weeks, every 4 weeks or monthly.

46. (canceled)

47. The method of claim 1, wherein the anti-IL-6 antibody is administered intravenously or subcutaneously.

48. The method of claim 1, wherein the subject:
(a) shows at least one of the following symptoms prior to treatment: barotrauma (volutrauma), pulmonary embolism (PE), pulmonary fibrosis, ventilator-associated pneumonia (VAP); gastrointestinal bleeding (ulcer), dysmotility, pneumoperitoneum, bacterial translocation; Hypoxic brain damage; abnormal heart rhythms, myocardial dysfunction; acute kidney failure, positive fluid balance; vascular injury, pneumothorax, tracheal injury/stenosis; malnutrition (catabolic state), electrolyte abnormalities; Atelectasis, blood clots, weakness in muscles used for breathing, stress ulcers, depression or other mental illness; single or multiple organ failure; pulmonary hypertension or increase in blood pressure in the main artery from the heart to the lungs; and/or
(b) is receiving supplemental oxygen prior to treatment.

49. (canceled)

50. The method of claim 48, wherein the subject is receiving supplemental oxygen prior to treatment, is not intubated, and is not on an invasive ventilator prior to treatment.

51. (canceled)

52. The method of claim 1, wherein the levels of IL-6 and/or CRP in the patient are detected and confirmed to be greater than 20 pg/mL prior to treatment.

53. (canceled)

54. (canceled)

55. The method of claim 1, wherein IL-6 and/or CRP levels in the subject are detected during and/or after treatment.

56. The method of claim 55, wherein a first 25 mg dose of clazakizumab is administered to the subject, and wherein a second dose is administered 24-48 h later if the C-reactive protein (CRP) level in the subject fails to decrease by at least 20%, 24-48 h after the first dose.

57.-60. (canceled)

61. The method of claim 1, wherein the subject is administered at least one additional therapeutic, and wherein the at least one additional therapeutic comprises one or more of corticosteroids; inhaled nitric oxide (NO); extracorporeal membrane oxygenation, or an immunosuppressive agent.

62. The method of claim 61, wherein the subject is further treated with:
(a) a Pneumocystis jiroveci pneumonia (PJP) therapeutic;
(b) a pulse steroid;
(c) one or more standard of care immunosuppression regimens;
(d) an antiviral, an antibiotic, or an immunosuppressive agent; and/or
(e) any of the following:
(i) azathioprine,
(ii) calcineurin inhibitors (CNIs),
(iii) mycophenolate mofetil (MMF)/mycophenolic acid (MPA),
(iv) mTOR inhibitors,
(v) low dose corticosteroids,
(vi) antihypertensive agents,
(vii) angiotensin II receptor blockers (ARBs),
(viii) cyclosporine,
(ix) antidiabetogenic agents, or
(x) or a combination of any of the previous.

63.-68. (canceled)

69. The method of claim 1, wherein:
(a) the subject has improved or normal lung function after treatment;
(b) the need for the subject to go on a ventilator is eliminated or the time the subject is on a ventilator is reduced as compared to placebo;
(c) compared to placebo the: (i) time to liberation from mechanical ventilation is reduced; (ii) time to durable fever resolution is reduced, (iii) time to durable improvement of oxygenation is reduced, (iv)C-reactive protein (CRP) response is reduced, (v) length of ICU stay is reduced, or (vi) number of subjects who survive 28 days post treatment is increased;
(d) the subject is hospitalized but does not exhibit pulmonary or respiratory difficulties requiring exogenous high levels of oxygen;
(e) (i) the number of COVID-19 infected subjects who develop ARDS is reduced; (ii) the onset of ARDS in COVID-19 infected subjects is slowed. and/or (iii) the method results in an ARDS condition in COVID-19 infected subjects which is less severe than in subjects not administered the anti-IL-6 antibody; and/or
(f) the subject: (i) is intubated and mechanically ventilated with acute respiratory distress syndrome (ARDS), and requires vasopressor support; (ii) has profound hypoxemia requiring supported by non-invasive ventilationory (NIV) support; (iii) is a solid organ recipient, optionally a kidney or heart recipient; (iv) shows sign of renal failure, optionally acute renal failure; (v) has elevated IL-6; (vi) has increased higher D-dimer levels, (vii) has increased fibrinogen levels and/or (viii) has increased ferritin levels; or a combination of any of the foregoing; wherein said increases, if present, are relative to median levels observed in normal, non-inflammatory persons.

70.-74. (canceled)


SEQUENCE LISTING (746 sequences)

SEQ ID NO: 1 - protein, 183 aa, Homo sapiens
VPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 2 - protein, 163 aa, Oryctolagus cuniculus
MDTRAPTQLLGLLLLWLPGARCAYDMTQTPASVSAAVGGTVTIKCQASQSINNELSWYQQKPGQRPKLLIYRASTLASGVSSRFKGSGSGTEFTLTISDLECADAATYYCQQGYSLRNIDNAFGGGTEVVVKRTVAAPSVFIFPPSDEQLKSGTASVVCLLNN

SEQ ID NO: 3 - protein, 166 aa, Oryctolagus cuniculus
METGLRWLLLVAVLKGVQCQSLEESGGRLVTPGTPLTLTCTASGFSLSNYYVTWVRQAPGKGLEWIGIIYGSDETAYATWAIGRFTISKTSTTVDLKMTSLTAADTATYFCARDDSSDWDAKFNLWGQGTLVTVSSASTKGPSVFPLAPSSKSTSGGTAALGCLVK

SEQ ID NO: 4 - protein, 11 aa, Oryctolagus cuniculus
QASQSINNELS

SEQ ID NO: 5 - protein, 7 aa, Oryctolagus cuniculus
RASTLAS

SEQ ID NO: 6 - protein, 12 aa, Oryctolagus cuniculus
QQGYSLRNIDNA

SEQ ID NO: 7 - protein, 5 aa, Oryctolagus cuniculus
NYYVT

SEQ ID NO: 8 - protein, 16 aa, Oryctolagus cuniculus
IIYGSDETAYATWAIG

SEQ ID NO: 9 - protein, 12 aa, Oryctolagus cuniculus
DDSSDWDAKFNL

SEQ ID NO: 10 - DNA, 491 bp, Oryctolagus cuniculus
atggacacgagggcccccactcagctgctggggctcctgctgctctggctcccaggtgccagatgtgcctatgatatgacccagactccagcctcggtgtctgcagctgtgggaggcacagtcaccatcaagtgccaggccagtcagagcattaacaatgaattatcctggtatcagcagaaaccagggcagcgtcccaagctcctgatctatagggcatccactctggcatctggggtctcatcgcggttcaaaggcagtggatctgggacagagttcactctcaccatcagcgacctggagtgtgccgatgctgccacttactactgtcaacagggttatagtctgaggaatattgataatgctttcggcggagggaccgaggtggtggtcaaacgtacggtagcggccccatctgtcttcatcttcccgccatctgatgagcagttgaaatctggaactgcctctgttgtgtgcctgctgaataactt

SEQ ID NO: 11 - DNA, 499 bp, Oryctolagus cuniculus
atggagactgggctgcgctggcttctcctggtcgctgtgctcaaaggtgtccagtgtcagtcgctggaggagtccgggggtcgcctggtcacgcctgggacacccctgacactcacctgcacagcctctggattctccctcagtaactactacgtgacctgggtccgccaggctccagggaaggggctggaatggatcggaatcatttatggtagtgatgaaacggcctacgcgacctgggcgataggccgattcaccatctccaaaacctcgaccacggtggatctgaaaatgaccagtctgacagccgcggacacggccacctatttctgtgccagagatgatagtagtgactgggatgcaaaatttaacttgtggggccaaggcaccctggtcaccgtctcgagcgcctccaccaagggcccatcggtcttccccctggcaccctcctccaagagcacctctgggggcacagcggccctgggctgcctggtcaagg

SEQ ID NO: 12 - DNA, 33 bp, Oryctolagus cuniculus
caggccagtcagagcattaacaatgaattatcc

SEQ ID NO: 13 - DNA, 21 bp, Oryctolagus cuniculus
agggcatccactctggcatct

SEQ ID NO: 14 - DNA, 36 bp, Oryctolagus cuniculus
caacagggttatagtctgaggaatattgataatgct

SEQ ID NO: 15 - DNA, 15 bp, Oryctolagus cuniculus
aactactacgtgacc

SEQ ID NO: 16 - DNA, 48 bp, Oryctolagus cuniculus
atcatttatggtagtgatgaaacggcctacgcgacctgggcgataggc

SEQ ID NO: 17 - DNA, 36 bp, Oryctolagus cuniculus
gatgatagtagtgactgggatgcaaaatttaacttg

SEQ ID NO: 18 - protein, 109 aa, Oryctolagus cuniculus
EVQLVESGGGLVQPGGSLRLSCAASGFSLSNYYVTWVRQAPGKGLEWVGIIYGSDETAYATWAIGRFTISRDNSKNTLYLQMNSLRAEDTAVYYCARDDSSDWDAKFNL

SEQ ID NO: 19 - protein, 109 aa, Oryctolagus cuniculus
EVQLVESGGGLVQPGGSLRLSCAASGFSLSNYYVTWVRQAPGKGLEWVGIIYGSDETAYATSAIGRFTISRDNSKNTLYLQMNSLRAEDTAVYYCARDDSSDWDAKFNL

SEQ ID NO: 20 - protein, 99 aa, Oryctolagus cuniculus
IQMTQSPSSLSASVGDRVTITCQASQSINNELSWYQQKPGKAPKLLIYRASTLASGVPSRFSGSGSGTDFTLTISSLQPDDFATYYCQQGYSLRNIDNA

SEQ ID NO: 21 - protein, 170 aa, Oryctolagus cuniculus
MDTRAPTQLLGLLLLWLPGARCAYDMTQTPASVEVAVGGTVTINCQASETIYSWLSWYQQKPGQPPKLLIYQASDLASGVPSRFSGSGAGTEYTLTISGVQCDDAATYYCQQGYSGSNVDNVFGGGTEVVVKRTVAAPSVFIFPPSDEQLKSGTASVVCLLNNFYPREAK

SEQ ID NO: 22 - protein, 167 aa, Oryctolagus cuniculus
METGLRWLLLVAVLKGVQCQEQLKESGGRLVTPGTPLTLTCTASGFSLNDHAMGWVRQAPGKGLEYIGFINSGGSARYASWAEGRFTISRTSTTVDLKMTSLTTEDTATYFCVRGGAVWSIHSFDPWGPGTLVTVSSASTKGPSVFPLAPSSKSTSGGTAALGCLVK

SEQ ID NO: 23 - protein, 11 aa, Oryctolagus cuniculus
QASETIYSWLS

SEQ ID NO: 24 - protein, 7 aa, Oryctolagus cuniculus
QASDLAS

SEQ ID NO: 25 - protein, 12 aa, Oryctolagus cuniculus
QQGYSGSNVDNV

SEQ ID NO: 26 - protein, 5 aa, Oryctolagus cuniculus
DHAMG

SEQ ID NO: 27 - protein, 16 aa, Oryctolagus cuniculus
FINSGGSARYASWAEG

SEQ ID NO: 28 - protein, 12 aa, Oryctolagus cuniculus
GGAVWSIHSFDP

SEQ ID NO: 29 - DNA, 511 bp, Oryctolagus cuniculus
atggacacgagggcccccactcagctgctggggctcctgctgctctggctcccaggtgccagatgtgcctatgatatgacccagactccagcctctgtggaggtagctgtgggaggcacagtcaccatcaattgccaggccagtgagaccatttacagttggttatcctggtatcagcagaagccagggcagcctcccaagctcctgatctaccaggcatccgatctggcatctggggtcccatcgcgattcagcggcagtggggctgggacagagtacactctcaccatcagcggcgtgcagtgtgacgatgctgccacttactactgtcaacagggttatagtggtagtaatgttgataatgttttcggcggagggaccgaggtggtggtcaaacgtacggtagcggccccatctgtcttcatcttcccgccatctgatgagcagttgaaatctggaactgcctctgttgtgtgcctgctgaataacttctatcccagagaggccaaag

SEQ ID NO: 30 - DNA, 501 bp, Oryctolagus cuniculus
atggagactgggctgcgctggcttctcctggtcgctgtgctcaaaggtgtccagtgtcaggagcagctgaaggagtccgggggtcgcctggtcacgcctgggacacccctgacacttacctgcacagcctctggattctccctcaatgaccatgcaatgggctgggtccgccaggctccagggaaggggctggaatacatcggattcattaatagtggtggtagcgcacgctacgcgagctgggcagaaggccgattcaccatctccagaacctcgaccacggtggatctgaaaatgaccagtctgacaaccgaggacacggccacctatttctgtgtcagagggggtgctgtttggagtattcatagttttgatccctggggcccagggaccctggtcaccgtctcgagcgcctccaccaagggcccatcggtcttccccctggcaccctcctccaagagcacctctgggggcacagcggccctgggctgcctggtcaag

SEQ ID NO: 31 - DNA, 33 bp, Oryctolagus cuniculus
caggccagtgagaccatttacagttggttatcc

SEQ ID NO: 32 - DNA, 21 bp, Oryctolagus cuniculus
caggcatccgatctggcatct

SEQ ID NO: 33 - DNA, 36 bp, Oryctolagus cuniculus
caacagggttatagtggtagtaatgttgataatgtt

SEQ ID NO: 34 - DNA, 15 bp, Oryctolagus cuniculus
gaccatgcaatgggc

SEQ ID NO: 35 - DNA, 48 bp, Oryctolagus cuniculus
ttcattaatagtggtggtagcgcacgctacgcgagctgggcagaaggc

SEQ ID NO: 36 - DNA, 36 bp, Oryctolagus cuniculus
gggggtgctgtttggagtattcatagttttgatccc

SEQ ID NO: 37 - protein, 165 aa, Oryctolagus cuniculus
MDTRAPTQLLGLLLLWLPGATFAAVLTQTPSPVSAAVGGTVSISCQASQSVYDNNYLSWFQQKPGQPPKLLIYGASTLASGVPSRFVGSGSGTQFTLTITDVQCDDAATYYCAGVYDDDSDNAFGGGTEVVVKRTVAAPSVFIFPPSDEQLKSGTASVVCLLNNF

SEQ ID NO: 38 - protein, 166 aa, Oryctolagus cuniculus
METGLRWLLLVAVLKGVQCQSLEESGGRLVTPGTPLTLTCTASGFSLSVYYMNWVRQAPGKGLEWIGFITMSDNINYASWAKGRFTISKTSTTVDLKMTSPTTEDTATYFCARSRGWGTMGRLDLWGPGTLVTVSSASTKGPSVFPLAPSSKSTSGGTAALGCLVK

SEQ ID NO: 39 - protein, 13 aa, Oryctolagus cuniculus
QASQSVYDNNYLS

SEQ ID NO: 40 - protein, 7 aa, Oryctolagus cuniculus
GASTLAS

SEQ ID NO: 41 - protein, 11 aa, Oryctolagus cuniculus
AGVYDDDSDNA

SEQ ID NO: 42 - protein, 5 aa, Oryctolagus cuniculus
VYYMN

SEQ ID NO: 43 - protein, 16 aa, Oryctolagus cuniculus
FITMSDNINYASWAKG

SEQ ID NO: 44 - protein, 12 aa, Oryctolagus cuniculus
SRGWGTMGRLDL

SEQ ID NO: 45 - DNA, 496 bp, Oryctolagus cuniculus
atggacacgagggcccccactcagctgctggggctcctgctgctctggctcccaggtgccacatttgccgccgtgctgacccagactccatctcccgtgtctgcagctgtgggaggcacagtcagcatcagttgccaggccagtcagagtgtttatgacaacaactacttatcctggtttcagcagaaaccagggcagcctcccaagctcctgatctatggtgcatccactctggcatctggggtcccatcgcggttcgtgggcagtggatctgggacacagttcactctcaccatcacagacgtgcagtgtgacgatgctgccacttactattgtgcaggcgtttatgatgatgatagtgataatgccttcggcggagggaccgaggtggtggtcaaacgtacggtagcggccccatctgtcttcatcttcccgccatctgatgagcagttgaaatctggaactgcctctgttgtgtgcctgctgaataacttct

SEQ ID NO: 46 - DNA, 499 bp, Oryctolagus cuniculus
atggagactgggctgcgctggcttctcctggtggctgtgctcaaaggtgtccagtgtcagtcgctggaggagtccgggggtcgcctggtcacccctgggacacccctgacactcacctgcacagcctctggattctccctcagtgtctactacatgaactgggtccgccaggctccagggaaggggctggaatggatcggattcattacaatgagtgataatataaattacgcgagctgggcgaaaggccgattcaccatctccaaaacctcgaccacggtggatctgaaaatgaccagtccgacaaccgaggacacggccacctatttctgtgccaggagtcgtggctggggtacaatgggtcggttggatctctggggcccaggcaccctcgtcaccgtctcgagcgcctccaccaagggcccatcggtcttccccctggcaccctcctccaagagcacctctgggggcacagcggccctgggctgcctggtcaagg

SEQ ID NO: 47 - DNA, 39 bp, Oryctolagus cuniculus
caggccagtcagagtgtttatgacaacaactacttatcc

SEQ ID NO: 48 - DNA, 21 bp, Oryctolagus cuniculus
ggtgcatccactctggcatct

SEQ ID NO: 49 - DNA, 33 bp, Oryctolagus cuniculus
gcaggcgtttatgatgatgatagtgataatgcc

SEQ ID NO: 50 - DNA, 15 bp, Oryctolagus cuniculus
gtctactacatgaac

SEQ ID NO: 51 - DNA, 48 bp, Oryctolagus cuniculus
ttcattacaatgagtgataatataaattacgcgagctgggcgaaaggc

SEQ ID NO: 52 - DNA, 36 bp, Oryctolagus cuniculus
agtcgtggctggggtacaatgggtcggttggatctc

SEQ ID NO: 53 - protein, 164 aa, Oryctolagus cuniculus
MDTRAPTQLLGLLLLWLPGAICDPVLTQTPSPVSAPVGGTVSISCQASQSVYENNYLSWFQQKPGQPPKLLIYGASTLDSGVPSRFKGSGSGTQFTLTITDVQCDDAATYYCAGVYDDDSDDAFGGGTEVVVKRTVAAPSVFIFPPSDEQLKSGTASVVCLLNN

SEQ ID NO: 54 - protein, 167 aa, Oryctolagus cuniculus
METGLRWLLLVAVLKGVQCQEQLKESGGGLVTPGGTLTLTCTASGFSLNAYYMNWVRQAPGKGLEWIGFITLNNNVAYANWAKGRFTFSKTSTTVDLKMTSPTPEDTATYFCARSRGWGAMGRLDLWGHGTLVTVSSASTKGPSVFPLAPSSKSTSGGTAALGCLVK

SEQ ID NO: 55 - protein, 13 aa, Oryctolagus cuniculus
QASQSVYENNYLS

SEQ ID NO: 56 - protein, 7 aa, Oryctolagus cuniculus
GASTLDS

SEQ ID NO: 57 - protein, 11 aa, Oryctolagus cuniculus
AGVYDDDSDDA

SEQ ID NO: 58 - protein, 5 aa, Oryctolagus cuniculus
AYYMN

SEQ ID NO: 59 - protein, 16 aa, Oryctolagus cuniculus
FITLNNNVAYANWAKG

SEQ ID NO: 60 - protein, 12 aa, Oryctolagus cuniculus
SRGWGAMGRLDL

SEQ ID NO: 61 - DNA, 494 bp, Oryctolagus cuniculus
atggacacgagggcccccactcagctgctggggctcctgctgctctggctcccaggtgccatatgtgaccctgtgctgacccagactccatctcccgtatctgcacctgtgggaggcacagtcagcatcagttgccaggccagtcagagtgtttatgagaacaactatttatcctggtttcagcagaaaccagggcagcctcccaagctcctgatctatggtgcatccactctggattctggggtcccatcgcggttcaaaggcagtggatctgggacacagttcactctcaccattacagacgtgcagtgtgacgatgctgccacttactattgtgcaggcgtttatgatgatgatagtgatgatgccttcggcggagggaccgaggtggtggtcaaacgtacggtagcggccccatctgtcttcatcttcccgccatctgatgagcagttgaaatctggaactgcctctgttgtgtgcctgctgaataactt

SEQ ID NO: 62 - DNA, 502 bp, Oryctolagus cuniculus
atggagactgggctgcgctggcttctcctggtggctgtgctcaaaggtgtccagtgtcaggagcagctgaaggagtccggaggaggcctggtaacgcctggaggaaccctgacactcacctgcacagcctctggattctccctcaatgcctactacatgaactgggtccgccaggctccagggaaggggctggaatggatcggattcattactctgaataataatgtagcttacgcgaactgggcgaaaggccgattcaccttctccaaaacctcgaccacggtggatctgaaaatgaccagtccgacacccgaggacacggccacctatttctgtgccaggagtcgtggctggggtgcaatgggtcggttggatctctggggccatggcaccctggtcaccgtctcgagcgcctccaccaagggcccatcggtcttccccctggcaccctcctccaagagcacctctgggggcacagcggccctgggctgcctggtcaagg

SEQ ID NO: 63 - DNA, 39 bp, Oryctolagus cuniculus
caggccagtcagagtgtttatgagaacaactatttatcc

SEQ ID NO: 64 - DNA, 21 bp, Oryctolagus cuniculus
ggtgcatccactctggattct

SEQ ID NO: 65 - DNA, 33 bp, Oryctolagus cuniculus
gcaggcgtttatgatgatgatagtgatgatgcc

SEQ ID NO: 66 - DNA, 15 bp, Oryctolagus cuniculus
gcctactacatgaac

SEQ ID NO: 67 - DNA, 48 bp, Oryctolagus cuniculus
ttcattactctgaataataatgtagcttacgcgaactgggcgaaaggc

SEQ ID NO: 68 - DNA, 36 bp, Oryctolagus cuniculus
agtcgtggctggggtgcaatgggtcggttggatctc

SEQ ID NO: 69 - protein, 164 aa, Oryctolagus cuniculus
MDTRAPTQLLGLLLLWLPGATFAQVLTQTPSPVSAAVGGTVTINCQASQSVDDNNWLGWYQQKRGQPPKYLIYSASTLASGVPSRFKGSGSGTQFTLTISDLECDDAATYYCAGGFSGNIFAFGGGTEVVVKRTVAAPSVFIFPPSDEQLKSGTASVVCLLNNF

SEQ ID NO: 70 - protein, 164 aa, Oryctolagus cuniculus
METGLRWLLLVAVLKGVQCQSVEESGGRLVTPGTPLTLTCTVSGFSLSSYAMSWVRQAPGKGLEWIGIIGGFGTTYYATWAKGRFTISKTSTTVDLRITSPTTEDTATYFCARGGPGNGGDIWGQGTLVTVSSASTKGPSVFPLAPSSKSTSGGTAALGCLVKD

SEQ ID NO: 71 - protein, 13 aa, Oryctolagus cuniculus
QASQSVDDNNWLG

SEQ ID NO: 72 - protein, 7 aa, Oryctolagus cuniculus
SASTLAS

SEQ ID NO: 73 - protein, 10 aa, Oryctolagus cuniculus
AGGFSGNIFA

SEQ ID NO: 74 - protein, 5 aa, Oryctolagus cuniculus
SYAMS

SEQ ID NO: 75 - protein, 16 aa, Oryctolagus cuniculus
IIGGFGTTYYATWAKG

SEQ ID NO: 76 - protein, 9 aa, Oryctolagus cuniculus
GGPGNGGDI

SEQ ID NO: 77 - DNA, 493 bp, Oryctolagus cuniculus
atggacacgagggcccccactcagctgctggggctcctgctgctctggctcccaggtgccacatttgcccaagtgctgacccagactccatcgcctgtgtctgcagctgtgggaggcacagtcaccatcaactgccaggccagtcagagtgttgatgataacaactggttaggctggtatcagcagaaacgagggcagcctcccaagtacctgatctattctgcatccactctggcatctggggtcccatcgcggttcaaaggcagtggatctgggacacagttcactctcaccatcagcgacctggagtgtgacgatgctgccacttactactgtgcaggcggttttagtggtaatatctttgctttcggcggagggaccgaggtggtggtcaaacgtacggtagcggccccatctgtcttcatcttcccgccatctgatgagcagttgaaatctggaactgcctctgttgtgtgcctgctgaataacttct

SEQ ID NO: 78 - DNA, 493 bp, Oryctolagus cuniculus
atggagactgggctgcgctggcttctcctggtcgctgtgctcaaaggtgtccagtgtcagtcggtggaggagtccgggggtcgcctggtcacgcctgggacacccctgacactcacctgcacagtctctggcttctccctcagtagctatgcaatgagctgggtccgccaggctccaggaaaggggctggagtggatcggaatcattggtggttttggtaccacatactacgcgacctgggcgaaaggccgattcaccatctccaaaacctcgaccacggtggatctgagaatcaccagtccgacaaccgaggacacggccacctatttctgtgccagaggtggtcctggtaatggtggtgacatctggggccaagggaccctggtcaccgtctcgagcgcctccaccaagggcccatcggtcttccccctggcaccctcctccaagagcacctctgggggcacagcggccctgggctgcctggtcaaggact

SEQ ID NO: 79 - DNA, 39 bp, Oryctolagus cuniculus
caggccagtcagagtgttgatgataacaactggttaggc

SEQ ID NO: 80 - DNA, 21 bp, Oryctolagus cuniculus
tctgcatccactctggcatct

SEQ ID NO: 81 - DNA, 30 bp, Oryctolagus cuniculus
gcaggcggttttagtggtaatatctttgct

SEQ ID NO: 82 - DNA, 15 bp, Oryctolagus cuniculus
agctatgcaatgagc

SEQ ID NO: 83 - DNA, 48 bp, Oryctolagus cuniculus
atcattggtggttttggtaccacatactacgcgacctgggcgaaaggc

SEQ ID NO: 84 - DNA, 27 bp, Oryctolagus cuniculus
ggtggtcctggtaatggtggtgacatc

SEQ ID NO: 85 - protein, 164 aa, Oryctolagus cuniculus
MDTRAPTQLLGLLLLWLPGATFAAVLTQTPSPVSVPVGGTVTIKCQSSQSVYNNFLSWYQQKPGQPPKLLIYQASKLASGVPDRFSGSGSGTQFTLTISGVQCDDAATYYCLGGYDDDADNAFGGGTEVVVKRTVAAPSVFIFPPSDEQLKSGTASVVCLLNNF

SEQ ID NO: 86 - protein, 170 aa, Oryctolagus cuniculus
METGLRWLLLVAVLKGVQCQSVEESGGRLVTPGTPLTLTCTVSGIDLSDYAMSWVRQAPGKGLEWIGIIYAGSGSTWYASWAKGRFTISKTSTTVDLKITSPTTEDTATYFCARDGYDDYGDFDRLDLWGPGTLVTVSSASTKGPSVFPLAPSSKSTSGGTAALGCLVKD

SEQ ID NO: 87 - protein, 12 aa, Oryctolagus cuniculus
QSSQSVYNNFLS

SEQ ID NO: 88 - protein, 7 aa, Oryctolagus cuniculus
QASKLAS

SEQ ID NO: 89 - protein, 11 aa, Oryctolagus cuniculus
LGGYDDDADNA

SEQ ID NO: 90 - protein, 5 aa, Oryctolagus cuniculus
DYAMS

SEQ ID NO: 91 - protein, 17 aa, Oryctolagus cuniculus
IIYAGSGSTWYASWAKG

SEQ ID NO: 92 - protein, 14 aa, Oryctolagus cuniculus
DGYDDYGDFDRLDL

SEQ ID NO: 93 - DNA, 492 bp, Oryctolagus cuniculus
atggacacgagggcccccactcagctgctggggctcctgctgctctggctcccaggtgccacatttgcagccgtgctgacccagacaccatcgcccgtgtctgtacctgtgggaggcacagtcaccatcaagtgccagtccagtcagagtgtttataataatttcttatcgtggtatcagcagaaaccagggcagcctcccaagctcctgatctaccaggcatccaaactggcatctggggtcccagataggttcagcggcagtggatctgggacacagttcactctcaccatcagcggcgtgcagtgtgacgatgctgccacttactactgtctaggcggttatgatgatgatgctgataatgctttcggcggagggaccgaggtggtggtcaaacgtacggtagcggccccatctgtcttcatcttcccgccatctgatgagcagttgaaatctggaactgcctctgttgtgtgcctgctgaataacttc

SEQ ID NO: 94 - DNA, 511 bp, Oryctolagus cuniculus
atggagactgggctgcgctggcttctcctggtcgctgtgctcaaaggtgtccagtgtcagtcggtggaggagtccgggggtcgcctggtcacgcctgggacacccctgacgctcacctgcacagtctctggaatcgacctcagtgactatgcaatgagctgggtccgccaggctccagggaaggggctggaatggatcggaatcatttatgctggtagtggtagcacatggtacgcgagctgggcgaaaggccgattcaccatctccaaaacctcgaccacggtggatctgaaaatcaccagtccgacaaccgaggacacggccacctatttctgtgccagagatggatacgatgactatggtgatttcgatcgattggatctctggggcccaggcaccctcgtcaccgtctcgagcgcctccaccaagggcccatcggtcttccccctggcaccctcctccaagagcacctctgggggcacagcggccctgggctgcctggtcaaggact

SEQ ID NO: 95 - DNA, 36 bp, Oryctolagus cuniculus
cagtccagtcagagtgtttataataatttcttatcg

SEQ ID NO: 96 - DNA, 21 bp, Oryctolagus cuniculus
caggcatccaaactggcatct

SEQ ID NO: 97 - DNA, 33 bp, Oryctolagus cuniculus
ctaggcggttatgatgatgatgctgataatgct

SEQ ID NO: 98 - DNA, 15 bp, Oryctolagus cuniculus
gactatgcaatgagc

SEQ ID NO: 99 - DNA, 51 bp, Oryctolagus cuniculus
atcatttatgctggtagtggtagcacatggtacgcgagctgggcgaaaggc

SEQ ID NO: 100 - DNA, 42 bp, Oryctolagus cuniculus
gatggatacgatgactatggtgatttcgatcgattggatctc

SEQ ID NO: 101 - protein, 164 aa, Oryctolagus cuniculus
MDTRAPTQLLGLLLLWLPGARCAYDMTQTPASVSAAVGGTVTIKCQASQSINNELSWYQQKSGQRPKLLIYRASTLASGVSSRFKGSGSGTEFTLTISDLECADAATYYCQQGYSLRNIDNAFGGGTEVVVKRTVAAPSVFIFPPSDEQLKSGTASVVCLLNNF

SEQ ID NO: 102 - protein, 166 aa, Oryctolagus cuniculus
METGLRWLLLVAVLSGVQCQSLEESGGRLVTPGTPLTLTCTASGFSLSNYYMTWVRQAPGKGLEWIGMIYGSDETAYANWAIGRFTISKTSTTVDLKMTSLTAADTATYFCARDDSSDWDAKFNLWGQGTLVTVSSASTKGPSVFPLAPSSKSTSGGTAALGCLVK

SEQ ID NO: 103 - protein, 11 aa, Oryctolagus cuniculus
QASQSINNELS

SEQ ID NO: 104 - protein, 7 aa, Oryctolagus cuniculus
RASTLAS

SEQ ID NO: 105 - protein, 12 aa, Oryctolagus cuniculus
QQGYSLRNIDNA

SEQ ID NO: 106 - protein, 5 aa, Oryctolagus cuniculus
NYYMT

SEQ ID NO: 107 - protein, 16 aa, Oryctolagus cuniculus
MIYGSDETAYANWAIG

SEQ ID NO: 108 - protein, 12 aa, Oryctolagus cuniculus
DDSSDWDAKFNL

SEQ ID NO: 109 - DNA, 492 bp, Oryctolagus cuniculus
atggacacgagggcccccactcagctgctggggctcctgctgctctggctcccaggtgccagatgtgcctatgatatgacccagactccagcctcggtgtctgcagctgtgggaggcacagtcaccatcaaatgccaggccagtcagagcattaacaatgaattatcctggtatcagcagaaatcagggcagcgtcccaagctcctgatctatagggcatccactctggcatctggggtctcatcgcggttcaaaggcagtggatctgggacagagttcactctcaccatcagcgacctggagtgtgccgatgctgccacttactactgtcaacagggttatagtctgaggaatattgataatgctttcggcggagggaccgaggtggtggtcaaacgtacggtagcggccccatctgtcttcatcttcccgccatctgatgagcagttgaaatctggaactgcctctgttgtgtgcctgctgaataacttc

SEQ ID NO: 110 - DNA, 499 bp, Oryctolagus cuniculus
atggagactgggctgcgctggcttctcctggtcgctgtgctctcaggtgtccagtgtcagtcgctggaggagtccgggggtcgcctggtcacgcctgggacacccctgacactcacctgcacagcctctggattctccctcagtaactactacatgacctgggtccgccaggctccagggaaggggctggaatggatcggaatgatttatggtagtgatgaaacagcctacgcgaactgggcgataggccgattcaccatctccaaaacctcgaccacggtggatctgaaaatgaccagtctgacagccgcggacacggccacctatttctgtgccagagatgatagtagtgactgggatgcaaaatttaacttgtggggccaagggaccctcgtcaccgtctcgagcgcctccaccaagggcccatcggtcttccccctggcaccctcctccaagagcacctctgggggcacagcggccctgggctgcctggtcaagg

SEQ ID NO: 111 - DNA, 33 bp, Oryctolagus cuniculus; identical to SEQ ID NO: 12

SEQ ID NO: 112 - DNA, 21 bp, Oryctolagus cuniculus
agggcatccactctggcatct

SEQ ID NO: 113 - DNA, 36 bp, Oryctolagus cuniculus; identical to SEQ ID NO: 14

SEQ ID NO: 114 - DNA, 15 bp, Oryctolagus cuniculus
aactactacatgacc

SEQ ID NO: 115 - DNA, 48 bp, Oryctolagus cuniculus
atgatttatggtagtgatgaaacagcctacgcgaactgggcgataggc

SEQ ID NO: 116 - DNA, 36 bp, Oryctolagus cuniculus; identical to SEQ ID NO: 17

SEQ ID NO: 117 - protein, 109 aa, Oryctolagus cuniculus
EVQLVESGGGLVQPGGSLRLSCAASGFSLSNYYMTWVRQAPGKGLEWVGMIYGSDETAYANWAIGRFTISRDNSKNTLYLQMNSLRAEDTAVYYCARDDSSDWDAKFNL

SEQ ID NO: 118 - protein, 109 aa, Oryctolagus cuniculus
EVQLVESGGGLVQPGGSLRLSCAASGFSLSNYYMTWVRQAPGKGLEWVGMIYGSDETAYANSAIGRFTISRDNSKNTLYLQMNSLRAEDTAVYYCARDDSSDWDAKFNL

SEQ ID NO: 119 - protein, 100 aa, Oryctolagus cuniculus
DIQMTQSPSTLSASVGDRVTITCQASQSINNELSWYQQKPGKAPKLLIYRASTLASGVPSRFSGSGSGTEFTLTISSLQPDDFATYYCQQGYSLRNIDNA

SEQ ID NO: 120 - protein, 16 aa, Oryctolagus cuniculus
IIYGSDETAYATSAIG

SEQ ID NO: 121 - protein, 16 aa, Oryctolagus cuniculus
MIYGSDETAYANSAIG

SEQ ID NO: 122 - protein, 123 aa, Oryctolagus cuniculus
MDTRAPTQLLGLLLLWLPGATFAAVLTQTPSPVSAAVGGTVTISCQSSQSVGNNQDLSWFQQRPGQPPKLLIYEISKLESGVPSRFSGSGSGTHFTLTISGVQCDDAATYYCLGGYDDDADNA

SEQ ID NO: 123 - protein, 128 aa, Oryctolagus cuniculus
METGLRWLLLVAVLKGVQCHSVEESGGRLVTPGTPLTLTCTVSGFSLSSRTMSWVRQAPGKGLEWIGYIWSGGSTYYATWAKGRFTISKTSTTVDLKITSPTTEDTATYFCARLGDTGGHAYATRLNL

SEQ ID NO: 124 - protein, 13 aa, Oryctolagus cuniculus
QSSQSVGNNQDLS

SEQ ID NO: 125 - protein, 7 aa, Oryctolagus cuniculus
EISKLES

SEQ ID NO: 126 - protein, 11 aa, Oryctolagus cuniculus
LGGYDDDADNA

SEQ ID NO: 127 - protein, 5 aa, Oryctolagus cuniculus
SRTMS

SEQ ID NO: 128 - protein, 16 aa, Oryctolagus cuniculus
YIWSGGSTYYATWAKG

SEQ ID NO: 129 - protein, 15 aa, Oryctolagus cuniculus
LGDTGGHAYATRLNL

SEQ ID NO: 130 - DNA, 369 bp, Oryctolagus cuniculus
atggacacgagggcccccactcagctgctggggctcctgctgctctggctcccaggtgccacatttgcagccgtgctgacccagacaccatcacccgtgtctgcagctgtgggaggcacagtcaccatcagttgccagtccagtcagagtgttggtaataaccaggacttatcctggtttcagcagagaccagggcagcctcccaagctcctgatctacgaaatatccaaactggaatctggggtcccatcgcggttcagcggcagtggatctgggacacacttcactctcaccatcagcggcgtacagtgtgacgatgctgccacttactactgtctaggcggttatgatgatgatgctgataatgct

SEQ ID NO: 131 - DNA, 384 bp, Oryctolagus cuniculus
atggagactgggctgcgctggcttctcctggtcgctgtgctcaaaggtgtccagtgtcactcggtggaggagtccgggggtcgcctggtcacgcctgggacacccctgacactcacctgcacagtctctggattctccctcagtagtcgtacaatgtcctgggtccgccaggctccagggaaggggctggagtggatcggatacatttggagtggtggtagcacatactacgcgacctgggcgaaaggccgattcaccatctccaaaacctcgaccacggtggatctgaaaatcaccagtccgacaaccgaggacacggccacctatttctgtgccagattgggcgatactggtggtcacgcttatgctactcgcttaaatctc

SEQ ID NO: 132 - DNA, 39 bp, Oryctolagus cuniculus
cagtccagtcagagtgttggtaataaccaggacttatcc

SEQ ID NO: 133 - DNA, 21 bp, Oryctolagus cuniculus
gaaatatccaaactggaatct

SEQ ID NO: 134 - DNA, 33 bp, Oryctolagus cuniculus; identical to SEQ ID NO: 97

SEQ ID NO: 135 - DNA, 15 bp, Oryctolagus cuniculus
agtcgtacaatgtcc

SEQ ID NO: 136 - DNA, 48 bp, Oryctolagus cuniculus
tacatttggagtggtggtagcacatactacgcgacctgggcgaaaggc

SEQ ID NO: 137 - DNA, 45 bp, Oryctolagus cuniculus
ttgggcgatactggtggtcacgcttatgctactcgcttaaatctc

SEQ ID NO: 138 - protein, 123 aa, Oryctolagus cuniculus
MDTRAPTQLLGLLLLWLPGATFAAVLTQTPSSVSAAVGGTVSISCQSSQSVYSNKYLAWYQQKPGQPPKLLIYWTSKLASGAPSRFSGSGSGTQFTLTISGVQCDDAATYYCLGAYDDDADNA

SEQ ID NO: 139 - protein, 126 aa, Oryctolagus cuniculus
METGLRWLLLVAVLKGVQCQSVEESGGRLVKPDETLTLTCTASGFSLEGGYMTWVRQAPGKGLEWIGISYDSGSTYYASWAKGRFTISKTSSTTVDLKMTSLTTEDTATYFCVRSLKYPTVTSDDL

SEQ ID NO: 140 - protein, 13 aa, Oryctolagus cuniculus
QSSQSVYSNKYLA

SEQ ID NO: 141 - protein, 7 aa, Oryctolagus cuniculus
WTSKLAS

SEQ ID NO: 142 - protein, 11 aa, Oryctolagus cuniculus
LGAYDDDADNA

SEQ ID NO: 143 - protein, 5 aa, Oryctolagus cuniculus
GGYMT

SEQ ID NO: 144 - protein, 16 aa, Oryctolagus cuniculus
ISYDSGSTYYASWAKG

SEQ ID NO: 145 - protein, 12 aa, Oryctolagus cuniculus
SLKYPTVTSDDL

SEQ ID NO: 146 - DNA, 369 bp, Oryctolagus cuniculus
atggacacgagggcccccactcagctgctggggctcctgctgctctggctcccaggtgccacatttgcagccgtgctgacccagacaccatcgtccgtgtctgcagctgtgggaggcacagtcagcatcagttgccagtccagtcagagtgtttatagtaataagtacctagcctggtatcagcagaaaccagggcagcctcccaagctcctgatctactggacatccaaactggcatctggggccccatcacggttcagcggcagtggatctgggacacaattcactctcaccatcagcggcgtgcagtgtgacgatgctgccacttactactgtctaggcgcttatgatgatgatgctgataatgct

SEQ ID NO: 147 - DNA, 378 bp, Oryctolagus cuniculus
atggagactgggctgcgctggcttctcctggtcgctgtgctcaaaggtgtccagtgtcagtcggtggaagagtccgggggtcgcctggtcaagcctgacgaaaccctgacactcacctgcacagcctctggattctccctggagggcggctacatgacctgggtccgccaggctccagggaaggggctggaatggatcggaatcagttatgatagtggtagcacatactacgcgagctgggcgaaaggccgattcaccatctccaagacctcgtcgaccacggtggatctgaaaatgaccagtctgacaaccgaggacacggccacctatttctgcgtcagatcactaaaatatcctactgttacttctgatgacttg

SEQ ID NO: 148 - DNA, 39 bp, Oryctolagus cuniculus
cagtccagtcagagtgtttatagtaataagtacctagcc

SEQ ID NO: 149 - DNA, 21 bp, Oryctolagus cuniculus
tggacatccaaactggcatct

SEQ ID NO: 150 - DNA, 33 bp, Oryctolagus cuniculus
ctaggcgcttatgatgatgatgctgataatgct

SEQ ID NO: 151 - DNA, 15 bp, Oryctolagus cuniculus
ggcggctacatgacc

SEQ ID NO: 152 - DNA, 48 bp, Oryctolagus cuniculus
atcagttatgatagtggtagcacatactacgcgagctgggcgaaaggc

SEQ ID NO: 153 - DNA, 36 bp, Oryctolagus cuniculus
tcactaaaatatcctactgttacttctgatgacttg

SEQ ID NO: 154 - protein, 123 aa, Oryctolagus cuniculus
MDTRAPTQLLGLLLLWLPGATFAAVLTQTPSPVSAAVGGTVTISCQSSQSVYNNNDLAWYQQKPGQPPKLLIYYASTLASGVPSRFKGSGSGTQFTLTISGVQCDDAAAYYCLGGYDDDADNA

SEQ ID NO: 155 - protein, 129 aa, Oryctolagus cuniculus
METGLRWLLLVAVLKGVQCQSVEESGGRLVTPGTPLTLTCTVSGLSLSSNTINWVRQAPGKGLEWIGYIWSGGSTYYASWVNGRFTISKTSTTVDLKITSPTTEDTATYFCARGGYASGGYPYATRLDL

SEQ ID NO: 156 - protein, 13 aa, Oryctolagus cuniculus
QSSQSVYNNNDLA

SEQ ID NO: 157 - protein, 7 aa, Oryctolagus cuniculus
YASTLAS

SEQ ID NO: 158 - protein, 11 aa, Oryctolagus cuniculus
LGGYDDDADNA

SEQ ID NO: 159 - protein, 5 aa, Oryctolagus cuniculus
SNTIN

SEQ ID NO: 160 - protein, 16 aa, Oryctolagus cuniculus
YIWSGGSTYYASWVNG

SEQ ID NO: 161 - protein, 16 aa, Oryctolagus cuniculus
GGYASGGYPYATRLDL

SEQ ID NO: 162 - DNA, 369 bp, Oryctolagus cuniculus
atggacacgagggcccccactcagctgctggggctcctgctgctctggctcccaggtgccacatttgcagccgtgctgacccagacaccatcacccgtgtctgcagctgtgggaggcacagtcaccatcagttgccagtccagtcagagtgtttataataataacgacttagcctggtatcagcagaaaccagggcagcctcctaaactcctgatctattatgcatccactctggcatctggggtcccatcgcggttcaaaggcagtggatctgggacacagttcactctcaccatcagcggcgtgcagtgtgacgatgctgccgcttactactgtctaggcggttatgatgatgatgctgataatgct

SEQ ID NO: 163 - DNA, 387 bp, Oryctolagus cuniculus
atggagactgggctgcgctggcttctcctggtcgctgtgctcaaaggtgtccagtgtcagtcggtggaggagtccgggggtcgcctggtcacgcctgggacacccctgacactcacctgcacagtatctggattatccctcagtagcaatacaataaactgggtccgccaggctccagggaaggggctggagtggatcggatacatttggagtggtggtagtacatactacgcgagctgggtgaatggtcgattcaccatctccaaaacctcgaccacggtggatctgaaaatcaccagtccgacaaccgaggacacggccacctatttctgtgccagagggggttacgctagtggtggttatccttatgccactcggttggatctc

SEQ ID NO: 164 - DNA, 39 bp, Oryctolagus cuniculus
cagtccagtcagagtgtttataataataacgacttagcc

SEQ ID NO: 165 - DNA, 21 bp, Oryctolagus cuniculus
tatgcatccactctggcatct

SEQ ID NO: 166 - DNA, 33 bp, Oryctolagus cuniculus; identical to SEQ ID NO: 97

SEQ ID NO: 167 - DNA, 15 bp, Oryctolagus cuniculus
agcaatacaataaac

SEQ ID NO: 168 - DNA, 48 bp, Oryctolagus cuniculus
tacatttggagtggtggtagtacatactacgcgagctgggtgaatggt

SEQ ID NO: 169 - DNA, 48 bp, Oryctolagus cuniculus
gggggttacgctagtggtggttatccttatgccactcggttggatctc

SEQ ID NO: 170 - protein, 123 aa, Oryctolagus cuniculus
MDTRAPTQLLGLLLLWLPGATFAAVLTQTPSSVSAAVGGTVTINCQSSQSVYNNDYLSWYQQRPGQRPKLLIYGASKLASGVPSRFKGSGSGKQFTLTISGVQCDDAATYYCLGDYDDDADNT

SEQ ID NO: 171 - protein, 123 aa, Oryctolagus cuniculus
METGLRWLLLVAVLKGVQCQSLEESGGRLVTPGTPLTLTCTVSGFTLSTNYYLSWVRQAPGKGLEWIGIIYPSGNTYCAKWAKGRFTISKTSSTTVDLKMTSPTTEDTATYFCARNYGGDESL

SEQ ID NO: 172 - protein, 13 aa, Oryctolagus cuniculus
QSSQSVYNNDYLS

SEQ ID NO: 173 - protein, 7 aa, Oryctolagus cuniculus
GASKLAS

SEQ ID NO: 174 - protein, 11 aa, Oryctolagus cuniculus
LGDYDDDADNT

SEQ ID NO: 175 - protein, 6 aa, Oryctolagus cuniculus
TNYYLS

SEQ ID NO: 176 - protein, 16 aa, Oryctolagus cuniculus
IIYPSGNTYCAKWAKG

SEQ ID NO: 177 - protein, 8 aa, Oryctolagus cuniculus
NYGGDESL

SEQ ID NO: 178 - DNA, 369 bp, Oryctolagus cuniculus
atggacacgagggcccccactcagctgctggggctcctgctgctctggctcccaggtgccacatttgcagccgtgctgacccagacaccatcctccgtgtctgcagctgtgggaggcacagtcaccatcaattgccagtccagtcagagtgtttataataacgactacttatcctggtatcaacagaggccagggcaacgtcccaagctcctaatctatggtgcttccaaactggcatctggggtcccgtcacggttcaaaggcagtggatctgggaaacagtttactctcaccatcagcggcgtgcagtgtgacgatgctgccacttactactgtctgggcgattatgatgatgatgctgataatact

SEQ ID NO: 179 - DNA, 369 bp, Oryctolagus cuniculus
atggagactgggctgcgctggcttctcctggtcgctgtgctcaaaggtgtccagtgtcagtcgctggaggagtccgggggtcgcctggtcacgcctgggacacccctgacactcacttgcacagtctctggattcaccctcagtaccaactactacctgagctgggtccgccaggctccagggaaggggctagaatggatcggaatcatttatcctagtggtaacacatattgcgcgaagtgggcgaaaggccgattcaccatctccaaaacctcgtcgaccacggtggatctgaaaatgaccagtccgacaaccgaggacacagccacgtatttctgtgccagaaattatggtggtgatgaaagtttg

SEQ ID NO: 180 - DNA, 39 bp, Oryctolagus cuniculus
cagtccagtcagagtgtttataataacgactacttatcc

SEQ ID NO: 181 - DNA, 21 bp, Oryctolagus cuniculus
ggtgcttccaaactggcatct

SEQ ID NO: 182 - DNA, 33 bp, Oryctolagus cuniculus
ctgggcgattatgatgatgatgctgataatact

SEQ ID NO: 183 - DNA, 18 bp, Oryctolagus cuniculus
accaactactacctgagc

SEQ ID NO: 184 - DNA, 48 bp, Oryctolagus cuniculus
atcatttatcctagtggtaacacatattgcgcgaagtgggcgaaaggc

SEQ ID NO: 185 - DNA, 24 bp, Oryctolagus cuniculus
aattatggtggtgatgaaagtttg

SEQ ID NO: 186 - protein, 119 aa, Oryctolagus cuniculus
MDTRAPTQLLGLLLLWLPGARCDVVMTQTPASVEAAVGGTVTIKCQASETIGNALAWYQQKSGQPPKLLIYKASKLASGVPSRFKGSGSGTEYTLTISDLECADAATYYCQWCYFGDSV

SEQ ID NO: 187 - protein, 128 aa, Oryctolagus cuniculus
METGLRWLLLVTVLKGVQCQEQLVESGGGLVQPEGSLTLTCTASGFDFSSGYYMCWVRQAPGKGLEWIACIFTITTNTYYASWAKGRFTISKTSSTTVTLQMTSLTAADTATYLCARGIYSDNNYYAL

SEQ ID NO: 188 - protein, 11 aa, Oryctolagus cuniculus
QASETIGNALA

SEQ ID NO: 189 - protein, 7 aa, Oryctolagus cuniculus
KASKLAS

SEQ ID NO: 190 - protein, 9 aa, Oryctolagus cuniculus
QWCYFGDSV

SEQ ID NO: 191 - protein, 6 aa, Oryctolagus cuniculus
SGYYMC

SEQ ID NO: 192 - protein, 17 aa, Oryctolagus cuniculus
CIFTITTNTYYASWAKG

SEQ ID NO: 193 - protein, 11 aa, Oryctolagus cuniculus
GIYSDNNYYAL

SEQ ID NO: 194 - DNA, 357 bp, Oryctolagus cuniculus
atggacacgagggcccccactcagctgctggggctcctgctgctctggctcccaggtgccagatgtgatgttgtgatgacccagactccagcctccgtggaggcagctgtgggaggcacagtcaccatcaagtgccaggccagtgagaccattggcaatgcattagcctggtatcagcagaaatcagggcagcctcccaagctcctgatctacaaggcatccaaactggcatctggggtcccatcgcggttcaaaggcagtggatctgggacagagtacactctcaccatcagcgacctggagtgtgccgatgctgccacttactactgtcaatggtgttattttggtgatagtgtt

SEQ ID NO: 195 - DNA, 384 bp, Oryctolagus cuniculus
atggagactgggctgcgctggcttctcctggtcactgtgctcaaaggtgtccagtgtcaggagcagctggtggagtccgggggaggcctggtccagcctgagggatccctgacactcacctgcacagcctctggattcgacttcagtagcggctactacatgtgctgggtccgccaggctccagggaaggggctggagtggatcgcgtgtattttcactattactactaacacttactacgcgagctgggcgaaaggccgattcaccatctccaagacctcgtcgaccacggtgactctgcaaatgaccagtctgacagccgcggacacggccacctatctctgtgcgagagggatttattctgataataattattatgccttg

SEQ ID NO: 196 - DNA, 33 bp, Oryctolagus cuniculus
caggccagtgagaccattggcaatgcattagcc

SEQ ID NO: 197 - DNA, 21 bp, Oryctolagus cuniculus
aaggcatccaaactggcatct

SEQ ID NO: 198 - DNA, 27 bp, Oryctolagus cuniculus
caatggtgttattttggtgatagtgtt

SEQ ID NO: 199 - DNA, 18 bp, Oryctolagus cuniculus
agcggctactacatgtgc

SEQ ID NO: 200 - DNA, 51 bp, Oryctolagus cuniculus
tgtattttcactattactactaacacttactacgcgagctgggcgaaaggc

SEQ ID NO: 201 - DNA, 33 bp, Oryctolagus cuniculus
gggatttattctgataataattattatgccttg

SEQ ID NO: 202 - protein, 119 aa, Oryctolagus cuniculus
MDTRAPTQLLGLLLLWLPGARCDVVMTQTPASVEAAVGGTVTIKCQASESIGNALAWYQQKPGQPPKLLIYKASTLASGVPSRFSGSGSGTEFTLTISGVQCADAAAYYCQWCYFGDSV

SEQ ID NO: 203 - protein, 128 aa, Oryctolagus cuniculus
METGLRWLLLVAVLKGVQCQQQLVESGGGLVKPGASLTLTCKASGFSFSSGYYMCWVRQAPGKGLESIACIFTITDNTYYANWAKGRFTISKPSSPTVTLQMTSLTAADTATYFCARGIYSTDNYYAL

SEQ ID NO: 204 - protein, 11 aa, Oryctolagus cuniculus
QASESIGNALA

SEQ ID NO: 205 - protein, 7 aa, Oryctolagus cuniculus
KASTLAS

SEQ ID NO: 206 - protein, 9 aa, Oryctolagus cuniculus
QWCYFGDSV

SEQ ID NO: 207 - protein, 6 aa, Oryctolagus cuniculus
SGYYMC

SEQ ID NO: 208 - protein, 17 aa, Oryctolagus cuniculus
CIFTITDNTYYANWAKG

SEQ ID NO: 209 - protein, 11 aa, Oryctolagus cuniculus
GIYSTDNYYAL

SEQ ID NO: 210 - DNA, 357 bp, Oryctolagus cuniculus
atggacacgagggcccccactcagctgctggggctcctgctgctctggctcccaggtgccagatgtgatgttgtgatgacccagactccagcctccgtggaggcagctgtgggaggcacagtcaccatcaagtgccaggccagtgagagcattggcaatgcattagcctggtatcagcagaaaccagggcagcctcccaagctcctgatctacaaggcatccactctggcatctggggtcccatcgcggttcagcggcagtggatctgggacagagttcactctcaccatcagcggcgtgcagtgtgccgatgctgccgcttactactgtcaatggtgttattttggtgatagtgtt

SEQ ID NO: 211 - DNA, 384 bp, Oryctolagus cuniculus
atggagactgggctgcgctggcttctcctggtcgctgtgctcaaaggtgtccagtgtcagcagcagctggtggagtccgggggaggcctggtcaagccgggggcatccctgacactcacctgcaaagcctctggattctccttcagtagcggctactacatgtgctgggtccgccaggctccagggaaggggctggagtcgatcgcatgcatttttactattactgataacacttactacgcgaactgggcgaaaggccgattcaccatctccaagccctcgtcgcccacggtgactctgcaaatgaccagtctgacagccgcggacacggccacctatttctgtgcgagggggatttattctactgataattattatgccttg

SEQ ID NO: 212 - DNA, 33 bp, Oryctolagus cuniculus
caggccagtgagagcattggcaatgcattagcc

SEQ ID NO: 213 - DNA, 21 bp, Oryctolagus cuniculus
aaggcatccactctggcatct

SEQ ID NO: 214 - DNA, 27 bp, Oryctolagus cuniculus
caatggtgttattttggtgatagtgtt

SEQ ID NO: 215 - DNA, 18 bp, Oryctolagus cuniculus
agcggctactacatgtgc

SEQ ID NO: 216 - DNA, 51 bp, Oryctolagus cuniculus
tgcatttttactattactgataacacttactacgcgaactgggcgaaaggc

SEQ ID NO: 217 - DNA, 33 bp, Oryctolagus cuniculus
gggatttattctactgataattattatgccttg

SEQ ID NO: 218 - protein, 123 aa, Oryctolagus cuniculus
MDTRAPTQLLGLLLLWLPGARCDVVMTQTPASVEAAVGGTVTIKCQASQSVSSYLNWYQQKPGQPPKLLIYRASTLESGVPSRFKGSGSGTEFTLTISDLECADAATYYCQCTYGTSSSYGAA

SEQ ID NO: 219 - protein, 133 aa, Oryctolagus cuniculus
METGLRWLLLVAVLKGVQCQSVEESGGRLVTPGTPLTLTCTVSGISLSSNAISWVRQAPGKGLEWIGIISYSGTTYYASWAKGRFTISKTSSTTVDLKITSPTTEDTATYFCARDDPTTVMVMLIPFGAGMDL

SEQ ID NO: 220 - protein, 11 aa, Oryctolagus cuniculus
QASQSVSSYLN

SEQ ID NO: 221 - protein, 7 aa, Oryctolagus cuniculus
RASTLES

SEQ ID NO: 222 - protein, 13 aa, Oryctolagus cuniculus
QCTYGTSSSYGAA

SEQ ID NO: 223 - protein, 5 aa, Oryctolagus cuniculus
SNAIS

SEQ ID NO: 224 - protein, 16 aa, Oryctolagus cuniculus
IISYSGTTYYASWAKG

SEQ ID NO: 225 - protein, 19 aa, Oryctolagus cuniculus
DDPTTVMVMLIPFGAGMDL

SEQ ID NO: 226 - DNA, 369 bp, Oryctolagus cuniculus
atggacacgagggcccccactcagctgctggggctcctgctgctctggctcccaggtgccagatgtgatgttgtgatgacccagactccagcctccgtggaggcagctgtgggaggcacagtcaccatcaagtgccaggccagtcagagcgttagtagctacttaaactggtatcagcagaaaccagggcagcctcccaagctcctgatctacagggcatccactctggaatctggggtcccatcgcggttcaaaggcagtggatctgggacagagttcactctcaccatcagcgacctggagtgtgccgatgctgccacttactactgtcaatgtacttatggtactagtagtagttatggtgctgct

SEQ ID NO: 227 - DNA, 399 bp, Oryctolagus cuniculus
atggagactgggctgcgctggcttctcctggtcgctgtgctcaaaggtgtccagtgtcagtcggtggaggagtccgggggtcgcctggtcacgcctgggacacccctgacactcacctgcaccgtctctggtatctccctcagtagcaatgcaataagctgggtccgccaggctccagggaaggggctggaatggatcggaatcattagttatagtggtaccacatactacgcgagctgggcgaaaggccgattcaccatctccaaaacctcgtcgaccacggtggatctgaaaatcactagtccgacaaccgaggacacggccacctacttctgtgccagagatgaccctacgacagttatggttatgttgataccttttggagccggcatggacctc

SEQ ID NO: 228 - DNA, 33 bp, Oryctolagus cuniculus
caggccagtcagagcgttagtagctacttaaac

SEQ ID NO: 229 - DNA, 21 bp, Oryctolagus cuniculus
agggcatccactctggaatct

SEQ ID NO: 230 - DNA, 39 bp, Oryctolagus cuniculus
caatgtacttatggtactagtagtagttatggtgctgct

SEQ ID NO: 231 - DNA, 15 bp, Oryctolagus cuniculus
agcaatgcaataagc

SEQ ID NO: 232 - DNA, 48 bp, Oryctolagus cuniculus
atcattagttatagtggtaccacatactacgcgagctgggcgaaaggc

SEQ ID NO: 233 - DNA, 57 bp, Oryctolagus cuniculus
gatgaccctacgacagttatggttatgttgataccttttggagccggcatggacctc

SEQ ID NO: 234 - protein, 125 aa, Oryctolagus cuniculus
MDTRAPTQLLGLLLLWLPGATFAQVLTQTASPVSAAVGGTVTINCQASQSVYKNNYLSWYQQKPGQPPKGLIYSASTLDSGVPLRFSGSGSGTQFTLTISDVQCDDAATYYCLGSYDCSSGDCYA

SEQ ID NO: 235 - protein, 119 aa, Oryctolagus cuniculus
METGLRWLLLVAVLKGVQCQSLEESGGDLVKPEGSLTLTCTASGFSFSSYWMCWVRQAPGKGLEWIACIVTGNGNTYYANWAKGRFTISKTSSTTVTLQMTSLTAADTATYFCAKAYDL

SEQ ID NO: 236 - protein, 13 aa, Oryctolagus cuniculus
QASQSVYKNNYLS

SEQ ID NO: 237 - protein, 7 aa, Oryctolagus cuniculus
SASTLDS

SEQ ID NO: 238 - protein, 13 aa, Oryctolagus cuniculus
LGSYDCSSGDCYA

SEQ ID NO: 239 - protein, 5 aa, Oryctolagus cuniculus
SYWMC

SEQ ID NO: 240 - protein, 17 aa, Oryctolagus cuniculus
CIVTGNGNTYYANWAKG

SEQ ID NO: 241 - protein, 4 aa, Oryctolagus cuniculus
AYDL

SEQ ID NO: 242 - DNA, 375 bp, Oryctolagus cuniculus
atggacacgagggcccccactcagctgctggggctcctgctgctctggctcccaggtgccacatttgcccaagtgctgacccagactgcatcgcccgtgtctgcagctgtgggaggcacagtcaccatcaactgccaggccagtcagagtgtttataagaacaactacttatcctggtatcagcagaaaccagggcagcctcccaaaggcctgatctattctgcatcgactctagattctggggtcccattgcggttcagcggcagtggatctgggacacagttcactctcaccatcagcgacgtgcagtgtgacgatgctgccacttactactgtctaggcagttatgattgtagtagtggtgattgttatgct

SEQ ID NO: 243 - DNA, 357 bp, Oryctolagus cuniculus
atggagactgggctgcgctggcttctcctggtcgctgtgctcaaaggtgtccagtgtcagtcgttggaggagtccgggggagacctggtcaagcctgagggatccctgacactcacctgcacagcctctggattctccttcagtagctactggatgtgctgggtccgccaggctccagggaaggggctggagtggatcgcatgcattgttactggtaatggtaacacttactacgcgaactgggcgaaaggccgattcaccatctccaaaacctcgtcgaccacggtgactctgcaaatgaccagtctgacagccgcggacacggccacctatttttgtgcgaaagcctatgacttg

SEQ ID NO: 244 - DNA, 39 bp, Oryctolagus cuniculus
caggccagtcagagtgtttataagaacaactacttatcc

SEQ ID NO: 245 - DNA, 21 bp, Oryctolagus cuniculus
tctgcatcgactctagattct

SEQ ID NO: 246 - DNA, 39 bp, Oryctolagus cuniculus
ctaggcagttatgattgtagtagtggtgattgttatgct

SEQ ID NO: 247 - DNA, 15 bp, Oryctolagus cuniculus
agctactggatgtgc

SEQ ID NO: 248 - DNA, 51 bp, Oryctolagus cuniculus
tgcattgttactggtaatggtaacacttactacgcgaactgggcgaaaggc

SEQ ID NO: 249 - DNA, 12 bp, Oryctolagus cuniculus
gcctatgacttg

SEQ ID NO: 250 - protein, 123 aa, Oryctolagus cuniculus
MDTRAPTQLLGLLLLWLPGSTFAAVLTQTPSPVSAAVGGTVSISCQASQSVYDNNYLSWYQQKPGQPPKLLIYGASTLASGVPSRFKGTGSGTQFTLTITDVQCDDAATYYCAGVFNDDSDDA

SEQ ID NO: 251 - protein, 125 aa, Oryctolagus cuniculus
METGLRWLLLVAVPKGVQCQSLEESGGRLVTPGTPLTLTCTLSGFSLSAYYMSWVRQAPGKGLEWIGFITLSDHISYARWAKGRFTISKTSTTVDLKMTSPTTEDTATYFCARSRGWGAMGRLDL

SEQ ID NO: 252 - protein, 13 aa, Oryctolagus cuniculus
QASQSVYDNNYLS

SEQ ID NO: 253 - protein, 7 aa, Oryctolagus cuniculus
GASTLAS

SEQ ID NO: 254 - protein, 11 aa, Oryctolagus cuniculus
AGVFNDDSDDA

SEQ ID NO: 255 - protein, 5 aa, Oryctolagus cuniculus
AYYMS

SEQ ID NO: 256 - protein, 16 aa, Oryctolagus cuniculus
FITLSDHISYARWAKG

SEQ ID NO: 257 - protein, 12 aa, Oryctolagus cuniculus
SRGWGAMGRLDL

SEQ ID NO: 258 - DNA, 369 bp, Oryctolagus cuniculus
atggacacgagggcccccactcagctgctggggctcctgctgctctggctcccaggttccacatttgccgccgtgctgacccagactccatctcccgtgtctgcagctgtgggaggcacagtcagcatcagttgccaggccagtcagagtgtttatgacaacaactatttatcctggtatcagcagaaaccaggacagcctcccaagctcctgatctatggtgcatccactctggcatctggggtcccatcgcggttcaaaggcacgggatctgggacacagttcactctcaccatcacagacgtgcagtgtgacgatgctgccacttactattgtgcaggcgtttttaatgatgatagtgatgatgcc

SEQ ID NO: 259 - DNA, 375 bp, Oryctolagus cuniculus
atggagactgggctgcgctggcttctcctggtcgctgtgcccaaaggtgtccagtgtcagtcgctggaggagtccgggggtcgcctggtcacgcctgggacacccctgacactcacctgcacactctctggattctccctcagtgcatactatatgagctgggtccgccaggctccagggaaggggctggaatggatcggattcattactctgagtgatcatatatcttacgcgaggtgggcgaaaggccgattcaccatctccaaaacctcgaccacggtggatctgaaaatgaccagtccgacaaccgaggacacggccacctatttctgtgccaggagtcgtggctggggtgcaatgggtcggttggatctc

SEQ ID NO: 260 - DNA, 39 bp, Oryctolagus cuniculus
caggccagtcagagtgtttatgacaacaactatttatcc

SEQ ID NO: 261 - DNA, 21 bp, Oryctolagus cuniculus
ggtgcatccactctggcatct

SEQ ID NO: 262 - DNA, 33 bp, Oryctolagus cuniculus
gcaggcgtttttaatgatgatagtgatgatgcc

SEQ ID NO: 263 - DNA, 15 bp, Oryctolagus cuniculus
gcatactatatgagc

SEQ ID NO: 264 - DNA, 48 bp, Oryctolagus cuniculus
ttcattactctgagtgatcatatatcttacgcgaggtgggcgaaaggc

SEQ ID NO: 265 - DNA, 36 bp, Oryctolagus cuniculus; identical to SEQ ID NO: 68

SEQ ID NO: 266 - protein, 123 aa, Oryctolagus cuniculus
MDTRAPTQLLGLLLLWLPGATFAAVLTQTPSPVSAAVGGTVTISCQASQSVYNNKNLAWYQQKSGQPPKLLIYWASTLASGVSSRFSGSGSGTQFTLTVSGVQCDDAATYYCLGVFDDDADNA

SEQ ID NO: 267 - protein, 121 aa, Oryctolagus cuniculus
METGLRWLLLVAVLKGVQCQSVEESGGRLVTPGTPLTLTCTASGFSLSSYSMTWVRQAPGKGLEYIGVIGTSGSTYYATWAKGRFTISRTSTTVALKITSPTTEDTATYFCVRSLSSITFL

SEQ ID NO: 268 - protein, 13 aa, Oryctolagus cuniculus
QASQSVYNNKNLA

SEQ ID NO: 269 - protein, 7 aa, Oryctolagus cuniculus
WASTLAS

SEQ ID NO: 270 - protein, 11 aa, Oryctolagus cuniculus
LGVFDDDADNA

SEQ ID NO: 271 - protein, 5 aa, Oryctolagus cuniculus
SYSMT

SEQ ID NO: 272 - protein, 16 aa, Oryctolagus cuniculus
VIGTSGSTYYATWAKG

SEQ ID NO: 273 - protein, 8 aa, Oryctolagus cuniculus
SLSSITFL

SEQ ID NO: 274 - DNA, 369 bp, Oryctolagus cuniculus
atggacacgagggcccccactcagctgctggggctcctgctgctctggctcccaggtgccacattcgcagccgtgctgacccagacaccatcgcccgtgtctgcggctgtgggaggcacagtcaccatcagttgccaggccagtcagagtgtttataacaacaaaaatttagcctggtatcagcagaaatcagggcagcctcccaagctcctgatctactgggcatccactctggcatctggggtctcatcgcggttcagcggcagtggatctgggacacagttcactctcaccgtcagcggcgtgcagtgtgacgatgctgccacttactactgtctaggcgtttttgatgatgatgctgataatgct

SEQ ID NO: 275 - DNA, 363 bp, Oryctolagus cuniculus
atggagactgggctgcgctggcttctcctggtcgctgtgctcaaaggtgtccaatgtcagtcggtggaggagtccgggggtcgcctggtcacgcctgggacacccctgacactcacctgcacagcctctggattctccctcagtagctactccatgacctgggtccgccaggctccagggaaggggctggaatatatcggagtcattggtactagtggtagcacatactacgcgacctgggcgaaaggccgattcaccatctccagaacctcgaccacggtggctctgaaaatcaccagtccgacaaccgaggacacggccacctatttctgtgtcaggagtctttcttctattactttcttg

SEQ ID NO: 276 - DNA, 39 bp, Oryctolagus cuniculus
caggccagtcagagtgtttataacaacaaaaatttagcc

SEQ ID NO: 277 - DNA, 21 bp, Oryctolagus cuniculus
tgggcatccactctggcatct

SEQ ID NO: 278 - DNA, 33 bp, Oryctolagus cuniculus
ctaggcgtttttgatgatgatgctgataatgct

SEQ ID NO: 279 - DNA, 15 bp, Oryctolagus cuniculus
agctactccatgacc

SEQ ID NO: 280 - DNA, 48 bp, Oryctolagus cuniculus
gtcattggtactagtggtagcacatactacgcgacctgggcgaaaggc

SEQ ID NO: 281 - DNA, 24 bp, Oryctolagus cuniculus
agtctttcttctattactttcttg

SEQ ID NO: 282 - protein, 120 aa, Oryctolagus cuniculus
MDTRAPTQLLGLLLLWLPGARCAFELTQTPASVEAAVGGTVTINCQASQNIYRYLAWYQQKPGQPPKFLIYLASTLASGVPSRFKGSGSGTEFTLTISDLECADAATYYCQSYYSSNSVA

SEQ ID NO: 283 - protein, 128 aa, Oryctolagus cuniculus
METGLRWLLLVAVLKGVQCQEQLVESGGDLVQPEGSLTLTCTASELDFSSGYWICWVRQVPGKGLEWIGCIYTGSSGSTFYASWAKGRFTISKTSSTTVTLQMTSLTAADTATYFCARGYSGFGYFKL

SEQ ID NO: 284 - protein, 11 aa, Oryctolagus cuniculus
QASQNIYRYLA

SEQ ID NO: 285 - protein, 7 aa, Oryctolagus cuniculus
LASTLAS

SEQ ID NO: 286 - protein, 10 aa, Oryctolagus cuniculus
QSYYSSNSVA

SEQ ID NO: 287 - protein, 6 aa, Oryctolagus cuniculus
SGYWIC

SEQ ID NO: 288 - protein, 18 aa, Oryctolagus cuniculus
CIYTGSSGSTFYASWAKG

SEQ ID NO: 289 - protein, 10 aa, Oryctolagus cuniculus
GYSGFGYFKL

SEQ ID NO: 290 - DNA, 360 bp, Oryctolagus cuniculus
atggacacgagggcccccactcagctgctggggctcctgctgctctggctcccaggtgccagatgtgcattcgaattgacccagactccagcctccgtggaggcagctgtgggaggcacagtcaccatcaattgccaggccagtcagaacatttatagatacttagcctggtatcagcagaaaccagggcagcctcccaagttcctgatctatctggcatctactctggcatctggggtcccatcgcggtttaaaggcagtggatctgggacagagttcactctcaccatcagcgacctggagtgtgccgatgctgccacttactactgtcaaagttattatagtagtaatagtgtcgct

SEQ ID NO: 291 - DNA, 384 bp, Oryctolagus cuniculus
atggagactgggctgcgctggcttctcctggtcgctgtgctcaaaggtgtccagtgtcaggagcagctggtggagtccgggggagacctggtccagcctgagggatccctgacactcacctgcacagcttctgagttagacttcagtagcggctactggatatgctgggtccgccaggttccagggaaggggctggagtggatcggatgcatttatactggtagtagtggtagcactttttacgcgagttgggcgaaaggccgattcaccatctccaaaacctcgtcgaccacggtgactctgcaaatgaccagtctgacagccgcggacacggccacctatttctgtgcgagaggttatagtggctttggttactttaagttg

SEQ ID NO: 292 - DNA, 33 bp, Oryctolagus cuniculus
caggccagtcagaacatttatagatacttagcc

SEQ ID NO: 293 - DNA, 21 bp, Oryctolagus cuniculus
ctggcatctactctggcatct

SEQ ID NO: 294 - DNA, 30 bp, Oryctolagus cuniculus
caaagttattatagtagtaatagtgtcgct

SEQ ID NO: 295 - DNA, 18 bp, Oryctolagus cuniculus
agcggctactggatatgc

SEQ ID NO: 296 - DNA, 54 bp, Oryctolagus cuniculus
tgcatttatactggtagtagtggtagcactttttacgcgagttgggcgaaaggc

SEQ ID NO: 297 - DNA, 30 bp, Oryctolagus cuniculus
ggttatagtggctttggttactttaagttg

SEQ ID NO: 298 - protein, 122 aa, Oryctolagus cuniculus
MDTRAPTQLLGLLLLWLPGARCAYDMTQTPASVEVAVGGTVTIKCQASEDIYRLLAWYQQKPGQPPKLLIYDSSDLASGVPSRFKGSGSGTEFTLAISGVQCDDAATYYCQQAWSYSDIDNA

SEQ ID NO: 299 - protein, 123 aa, Oryctolagus cuniculus
METGLRWLLLVAVLKGVQCQSVEESGGRLVTPGTPLTLTCTASGFSLSSYYMSWVRQAPGKGLEWIGIITTSGNTFYASWAKGRLTISRTSTTVDLKITSPTTEDTATYFCARTSDIFYYRNL

SEQ ID NO: 300 - protein, 11 aa, Oryctolagus cuniculus
QASEDIYRLLA

SEQ ID NO: 301 - protein, 7 aa, Oryctolagus cuniculus
DSSDLAS

SEQ ID NO: 302 - protein, 12 aa, Oryctolagus cuniculus
QQAWSYSDIDNA

SEQ ID NO: 303 - protein, 5 aa, Oryctolagus cuniculus
SYYMS

SEQ ID NO: 304 - protein, 16 aa, Oryctolagus cuniculus
IITTSGNTFYASWAKG

SEQ ID NO: 305 - protein, 10 aa, Oryctolagus cuniculus
TSDIFYYRNL

SEQ ID NO: 306 - DNA, 366 bp, Oryctolagus cuniculus
atggacacgagggcccccactcagctgctggggctcctgctgctctggctcccaggtgccagatgtgcctatgatatgacccagactccagcctctgtggaggtagctgtgggaggcacagtcaccatcaagtgccaggccagtgaggacatttataggttattggcctggtatcaacagaaaccagggcagcctcccaagctcctgatctatgattcatccgatctggcatctggggtcccatcgcggttcaaaggcagtggatctgggacagagttcactctcgccatcagcggtgtgcagtgtgacgatgctgccacttactactgtcaacaggcttggagttatagtgatattgataatgct

SEQ ID NO: 307 - DNA, 369 bp, Oryctolagus cuniculus
atggagactgggctgcgctggcttctcctggtcgctgtgctcaaaggtgtccagtgtcagtcggtggaggagtccgggggtcgcctggtcacgccggggacacccctgacactcacctgcacagcctctggattctccctcagtagctactacatgagctgggtccgccaggctccagggaaggggctggaatggatcggaatcattactactagtggtaatacattttacgcgagctgggcgaaaggccggctcaccatctccagaacctcgaccacggtggatctgaaaatcaccagtccgacaaccgaggacacggccacctatttctgtgccagaacttctgatattttttattatcgtaacttg

SEQ ID NO: 308 - DNA, 33 bp, Oryctolagus cuniculus
caggccagtgaggacatttataggttattggcc

SEQ ID NO: 309 - DNA, 21 bp, Oryctolagus cuniculus
gattcatccgatctggcatct

SEQ ID NO: 310 - DNA, 36 bp, Oryctolagus cuniculus
caacaggcttggagttatagtgatattgataatgct

SEQ ID NO: 311 - DNA, 15 bp, Oryctolagus cuniculus
agctactacatgagc

SEQ ID NO: 312 - DNA, 48 bp, Oryctolagus cuniculus
atcattactactagtggtaatacattttacgcgagctgggcgaaaggc

SEQ ID NO: 313 - DNA, 30 bp, Oryctolagus cuniculus
acttctgatattttttattatcgtaacttg

SEQ ID NO: 314 - protein, 123 aa, Oryctolagus cuniculus
MDTRAPTQLLGLLLLWLPGATFAAVLTQTASPVSAAVGATVTINCQSSQSVYNDMDLAWFQQKPGQPPKLLIYSASTLASGVPSRFSGSGSGTEFTLTISGVQCDDAATYYCLGAFDDDADNT

SEQ ID NO: 315 - protein, 129 aa, Oryctolagus cuniculus
METGLRWLLLVAVLKGVQCQSVEESGGRLVTPGTPLTLTCTVSGFSLTRHAITWVRQAPGKGLEWIGCIWSGGSTYYATWAKGRFTISKTSTTVDLRITSPTTEDTATYFCARVIGDTAGYAYFTGLDL

SEQ ID NO: 316 - protein, 13 aa, Oryctolagus cuniculus
QSSQSVYNDMDLA

SEQ ID NO: 317 - protein, 7 aa, Oryctolagus cuniculus
SASTLAS

SEQ ID NO: 318 - protein, 11 aa, Oryctolagus cuniculus
LGAFDDDADNT

SEQ ID NO: 319 - protein, 5 aa, Oryctolagus cuniculus
RHAIT

SEQ ID NO: 320 - protein, 16 aa, Oryctolagus cuniculus
CIWSGGSTYYATWAKG

SEQ ID NO: 321 - protein, 16 aa, Oryctolagus cuniculus
VIGDTAGYAYFTGLDL

SEQ ID NO: 322 - DNA, 369 bp, Oryctolagus cuniculus
atggacacgagggcccccactcagctgctggggctcctgctgctctggctcccaggtgccacgtttgcagccgtgctgacccagactgcatcacccgtgtctgccgctgtgggagccacagtcaccatcaactgccagtccagtcagagtgtttataatgacatggacttagcctggtttcagcagaaaccagggcagcctcccaagctcctgatctattctgcatccactctggcatctggggtcccatcgcggttcagcggcagtggatctgggacagagttcactctcaccatcagcggcgtgcagtgtgacgatgctgccacttactactgtctaggcgcttttgatgatgatgctgataatact

SEQ ID NO: 323 - DNA, 387 bp, Oryctolagus cuniculus
atggagactgggctgcgctggcttctcctggtcgctgtgctcaaaggtgtccagtgtcagtcggtggaggagtccgggggtcgcctggtcacgcctgggacacccctgacactcacctgcacagtctctggattctccctcactaggcatgcaataacctgggtccgccaggctccagggaaggggctggaatggatcggatgcatttggagtggtggtagcacatactacgcgacctgggcgaaaggccgattcaccatctccaaaacctcgaccacggtggatctcagaatcaccagtccgacaaccgaggacacggccacctacttctgtgccagagtcattggcgatactgctggttatgcttattttacggggcttgacttg

SEQ ID NO: 324 - DNA, 39 bp, Oryctolagus cuniculus
cagtccagtcagagtgtttataatgacatggacttagcc

SEQ ID NO: 325 - DNA, 21 bp, Oryctolagus cuniculus
tctgcatccactctggcatct

SEQ ID NO: 326 - DNA, 33 bp, Oryctolagus cuniculus
ctaggcgcttttgatgatgatgctgataatact

SEQ ID NO: 327 - DNA, 15 bp, Oryctolagus cuniculus
aggcatgcaataacc

SEQ ID NO: 328 - DNA, 48 bp, Oryctolagus cuniculus
tgcatttggagtggtggtagcacatactacgcgacctgggcgaaaggc

SEQ ID NO: 329 - DNA, 48 bp, Oryctolagus cuniculus
gtcattggcgatactgctggttatgcttattttacggggcttgacttg

SEQ ID NO: 330 - protein, 121 aa, Oryctolagus cuniculus
MDTRAPTQLLGLLLLWLPGARCAYDMTQTPASVEVAVGGTVTIKCQASQSVYNWLSWYQQKPGQPPKLLIYTASSLASGVPSRFSGSGSGTEFTLTISGVECADAATYYCQQGYTSDVDNV

SEQ ID NO: 331 - protein, 130 aa, Oryctolagus cuniculus
METGLRWLLLVAVLKGVQCQSLEEAGGRLVTPGTPLTLTCTVSGIDLSSYAMGWVRQAPGKGLEYIGIISSSGSTYYATWAKGRFTISQASSTTVDLKITSPTTEDSATYFCARGGAGSGGVWLLDGFDP

SEQ ID NO: 332 - protein, 11 aa, Oryctolagus cuniculus
QASQSVYNWLS

SEQ ID NO: 333 - protein, 7 aa, Oryctolagus cuniculus
TASSLAS

SEQ ID NO: 334 - protein, 11 aa, Oryctolagus cuniculus
QQGYTSDVDNV

SEQ ID NO: 335 - protein, 5 aa, Oryctolagus cuniculus
SYAMG

SEQ ID NO: 336 - protein, 16 aa, Oryctolagus cuniculus
IISSSGSTYYATWAKG

SEQ ID NO: 337 - protein, 16 aa, Oryctolagus cuniculus
GGAGSGGVWLLDGFDP

SEQ ID NO: 338 - DNA, 363 bp, Oryctolagus cuniculus
atggacacgagggcccccactcagctgctggggctcctgctgctctggctcccaggtgccagatgtgcctatgatatgacccagactccagcctctgtggaggtagctgtgggaggcacagtcaccatcaagtgccaggccagtcagagtgtttataattggttatcctggtatcagcagaaaccagggcagcctcccaagctcctgatctatactgcatccagtctggcatctggggtcccatcgcggttcagtggcagtggatctgggacagagttcactctcaccatcagcggcgtggagtgtgccgatgctgccacttactactgtcaacagggttatactagtgatgttgataatgtt

SEQ ID NO: 339 - DNA, 390 bp, Oryctolagus cuniculus
atggagactgggctgcgctggcttctcctggtcgctgtgctcaaaggtgtccagtgtcagtcgctggaggaggccgggggtcgcctggtcacgcctgggacacccctgacactcacctgcacagtctctggaatcgacctcagtagctatgcaatgggctgggtccgccaggctccagggaaggggctggaatacatcggaatcattagtagtagtggtagcacatactacgcgacctgggcgaaaggccgattcaccatctcacaagcctcgtcgaccacggtggatctgaaaattaccagtccgacaaccgaggactcggccacatatttctgtgccagagggggtgctggtagtggtggtgtttggctgcttgatggttttgatccc

SEQ ID NO: 340 - DNA, 33 bp, Oryctolagus cuniculus
caggccagtcagagtgtttataattggttatcc

SEQ ID NO: 341 - DNA, 21 bp, Oryctolagus cuniculus
actgcatccagtctggcatct

SEQ ID NO: 342 - DNA, 33 bp, Oryctolagus cuniculus
caacagggttatactagtgatgttgataatgtt

SEQ ID NO: 343 - DNA, 15 bp, Oryctolagus cuniculus
agctatgcaatgggc

SEQ ID NO: 344 - DNA, 48 bp, Oryctolagus cuniculus
atcattagtagtagtggtagcacatactacgcgacctgggcgaaaggc

SEQ ID NO: 345 - DNA, 48 bp, Oryctolagus cuniculus
gggggtgctggtagtggtggtgtttggctgcttgatggttttgatccc

SEQ ID NO: 346 - protein, 123 aa, Oryctolagus cuniculus
MDTRAPTQLLGLLLLWLPGAKCADVVMTQTPASVSAAVGGTVTINCQASENIYNWLAWYQQKPGQPPKLLIYTVGDLASGVSSRFKGSGSGTEFTLTISDLECADAATYYCQQGYSSSYVDNV

SEQ ID NO: 347 - protein, 130 aa, Oryctolagus cuniculus
METGLRWLLLVAVLKGVQCQEQLKESGGRLVTPGTPLTLTCTVSGFSLNDYAVGWFRQAPGKGLEWIGYIRSSGTTAYATWAKGRFTISATSTTVDLKITSPTTEDTATYFCARGGAGSSGVWILDGFAP

SEQ ID NO: 348 - protein, 11 aa, Oryctolagus cuniculus
QASENIYNWLA

SEQ ID NO: 349 - protein, 7 aa, Oryctolagus cuniculus
TVGDLAS

SEQ ID NO: 350 - protein, 12 aa, Oryctolagus cuniculus
QQGYSSSYVDNV

SEQ ID NO: 351 - protein, 5 aa, Oryctolagus cuniculus
DYAVG

SEQ ID NO: 352 - protein, 16 aa, Oryctolagus cuniculus
YIRSSGTTAYATWAKG

SEQ ID NO: 353 - protein, 16 aa, Oryctolagus cuniculus
GGAGSSGVWILDGFAP

SEQ ID NO: 354 - DNA, 369 bp, Oryctolagus cuniculus
atggacacgagggcccccactcagctgctggggctcctgctgctctggctcccaggtgccaaatgtgccgatgttgtgatgacccagactccagcctccgtgtctgcagctgtgggaggcacagtcaccatcaattgccaggccagtgagaacatttataattggttagcctggtatcagcagaaaccagggcagcctcccaagctcctgatctatactgtaggcgatctggcatctggggtctcatcgcggttcaaaggcagtggatctgggacagagttcactctcaccatcagcgacctggagtgtgccgatgctgccacttactattgtcaacagggttatagtagtagttatgttgataatgtt

SEQ ID NO: 355 - DNA, 390 bp, Oryctolagus cuniculus
atggagactgggctgcgctggcttctcctggtcgctgtgctcaaaggtgtccagtgtcaggagcagctgaaggagtccgggggtcgcctggtcacgcctgggacacccctgacactcacctgcacagtctctggattctccctcaatgactatgcagtgggctggttccgccaggctccagggaaggggctggaatggatcggatacattcgtagtagtggtaccacagcctacgcgacctgggcgaaaggccgattcaccatctccgctacctcgaccacggtggatctgaaaatcaccagtccgacaaccgaggacacggccacctatttctgtgccagagggggtgctggtagtagtggtgtgtggatccttgatggttttgctccc

SEQ ID NO: 356 - DNA, 33 bp, Oryctolagus cuniculus
caggccagtgagaacatttataattggttagcc

SEQ ID NO: 357 - DNA, 21 bp, Oryctolagus cuniculus
actgtaggcgatctggcatct

SEQ ID NO: 358 - DNA, 36 bp, Oryctolagus cuniculus
caacagggttatagtagtagttatgttgataatgtt

SEQ ID NO: 359 - DNA, 15 bp, Oryctolagus cuniculus
gactatgcagtgggc

SEQ ID NO: 360 - DNA, 48 bp, Oryctolagus cuniculus
tacattcgtagtagtggtaccacagcctacgcgacctgggcgaaaggc

SEQ ID NO: 361 - DNA, 48 bp, Oryctolagus cuniculus
gggggtgctggtagtagtggtgtgtggatccttgatggttttgctccc

SEQ ID NO: 362 - protein, 121 aa, Oryctolagus cuniculus
MDTRAPTQLLGLLLLWLPGATFAQVLTQTPSSVSAAVGGTVTINCQASQSVYQNNYLSWFQQKPGQPPKLLIYGAATLASGVPSRFKGSGSGTQFTLTISDLECDDAATYYCAGAYRDVDS

SEQ ID NO: 363 - protein, 130 aa, Oryctolagus cuniculus
METGLRWLLLVAVLKGVQCQSLEESGGDLVKPGASLTLTCTASGFSFTSTYYIYWVRQAPGKGLEWIACIDAGSSGSTYYATWVNGRFTISKTSSTTVTLQMTSLTAADTATYFCAKWDYGGNVGWGYDL

SEQ ID NO: 364 - protein, 13 aa, Oryctolagus cuniculus
QASQSVYQNNYLS

SEQ ID NO: 365 - protein, 7 aa, Oryctolagus cuniculus
GAATLAS

SEQ ID NO: 366 - protein, 9 aa, Oryctolagus cuniculus
AGAYRDVDS

SEQ ID NO: 367 - protein, 6 aa, Oryctolagus cuniculus
STYYIY

SEQ ID NO: 368 - protein, 18 aa, Oryctolagus cuniculus
CIDAGSSGSTYYATWVNG

SEQ ID NO: 369 - protein, 13 aa, Oryctolagus cuniculus
WDYGGNVGWGYDL

SEQ ID NO: 370 - DNA, 363 bp, Oryctolagus cuniculus
atggacacgagggcccccactcagctgctggggctcctgctgctctggctcccaggtgccacatttgctcaagtgctgacccagactccatcctccgtgtctgcagctgtgggaggcacagtcaccatcaattgccaggccagtcagagtgtttatcagaacaactacttatcctggtttcagcagaaaccagggcagcctcccaagctcctgatctatggtgcggccactctggcatctggggtcccatcgcggttcaaaggcagtggatctgggacacagttcactctcaccatcagcgacctggagtgtgacgatgctgccacttactactgtgcaggcgcttatagggatgtggattct

SEQ ID NO: 371 - DNA, 390 bp, Oryctolagus cuniculus
atggagactgggctgcgctggcttctcctggtcgctgtgctcaaaggtgtccagtgtcagtcgttggaggagtccgggggagacctggtcaagcctggggcatccctgacactcacctgcacagcctctggattctcctttactagtacctactacatctactgggtccgccaggctccagggaaggggctggagtggatcgcatgtattgatgctggtagtagtggtagcacttactacgcgacctgggtgaatggccgattcaccatctccaaaacctcgtcgaccacggtgactctgcaaatgaccagtctgacagccgcggacacggccacctatttctgtgcgaaatgggattatggtggtaatgttggttggggttatgacttg

SEQ ID NO: 372 - DNA, 39 bp, Oryctolagus cuniculus
caggccagtcagagtgtttatcagaacaactacttatcc

SEQ ID NO: 373 - DNA, 21 bp, Oryctolagus cuniculus
ggtgcggccactctggcatct

SEQ ID NO: 374 - DNA, 27 bp, Oryctolagus cuniculus
gcaggcgcttatagggatgtggattct

SEQ ID NO: 375 - DNA, 18 bp, Oryctolagus cuniculus
agtacctactacatctac

SEQ ID NO: 376 - DNA, 54 bp, Oryctolagus cuniculus
tgtattgatgctggtagtagtggtagcacttactacgcgacctgggtgaatggc

SEQ ID NO: 377 - DNA, 39 bp, Oryctolagus cuniculus
tgggattatggtggtaatgttggttggggttatgacttg

SEQ ID NO: 378 - protein, 120 aa, Oryctolagus cuniculus
MDTRAPTQLLGLLLLWLPGARCAFELTQTPSSVEAAVGGTVTIKCQASQSISSYLAWYQQKPGQPPKFLIYRASTLASGVPSRFKGSGSGTEFTLTISDLECADAATYYCQSYYDSVSNP

SEQ ID NO: 379 - protein, 127 aa, Oryctolagus cuniculus
METGLRWLLLVAVLKGVQCQSLEESGGDLVKPEGSLTLTCKASGLDLGTYWFMCWVRQAPGKGLEWIACIYTGSSGSTFYASWVNGRFTISKTSSTTVTLQMTSLTAADTATYFCARGYSGYGYFKL

SEQ ID NO: 380 - protein, 11 aa, Oryctolagus cuniculus
QASQSISSYLA

SEQ ID NO: 381 - protein, 7 aa, Oryctolagus cuniculus
RASTLAS

SEQ ID NO: 382 - protein, 10 aa, Oryctolagus cuniculus
QSYYDSVSNP

SEQ ID NO: 383 - protein, 6 aa, Oryctolagus cuniculus
TYWFMC

SEQ ID NO: 384 - protein, 18 aa, Oryctolagus cuniculus
CIYTGSSGSTFYASWVNG

SEQ ID NO: 385 - protein, 10 aa, Oryctolagus cuniculus
GYSGYGYFKL

SEQ ID NO: 386 - DNA, 360 bp, Oryctolagus cuniculus
atggacacgagggcccccactcagctgctggggctcctgctgctctggctcccaggtgccagatgtgcattcgaattgacccagactccatcctccgtggaggcagctgtgggaggcacagtcaccatcaagtgccaggccagtcagagcattagtagttacttagcctggtatcagcagaaaccagggcagcctcccaagttcctgatctacagggcgtccactctggcatctggggtcccatcgcgattcaaaggcagtggatctgggacagagttcactctcaccatcagcgacctggagtgtgccgatgctgccacttactactgtcaaagctattatgatagtgtttcaaatcct

SEQ ID NO: 387 - DNA, 381 bp, Oryctolagus cuniculus
atggagactgggctgcgctggcttctcctggtcgctgtgctcaaaggtgtccagtgtcagtcgttggaggagtccgggggagacctggtcaagcctgagggatccctgacactcacctgcaaagcctctggactcgacctcggtacctactggttcatgtgctgggtccgccaggctccagggaaggggctggagtggatcgcttgtatttatactggtagtagtggttccactttctacgcgagctgggtgaatggccgattcaccatctccaaaacctcgtcgaccacggtgactctgcaaatgaccagtctgacagccgcggacacggccacttatttttgtgcgagaggttatagtggttatggttattttaagttg

SEQ ID NO: 388 - DNA, 33 bp, Oryctolagus cuniculus
caggccagtcagagcattagtagttacttagcc

SEQ ID NO: 389 - DNA, 21 bp, Oryctolagus cuniculus
agggcgtccactctggcatct

SEQ ID NO: 390 - DNA, 30 bp, Oryctolagus cuniculus
caaagctattatgatagtgtttcaaatcct

SEQ ID NO: 391 - DNA, 18 bp, Oryctolagus cuniculus
acctactggttcatgtgc

SEQ ID NO: 392 - DNA, 54 bp, Oryctolagus cuniculus
tgtatttatactggtagtagtggttccactttctacgcgagctgggtgaatggc

SEQ ID NO: 393 - DNA, 30 bp, Oryctolagus cuniculus
ggttatagtggttatggttattttaagttg

SEQ ID NO: 394 - protein, 124 aa, Oryctolagus cuniculus
MDTRAPTQLLGLLLLWLPGVTFAIEMTQSPFSVSAAVGGTVSISCQASQSVYKNNQLSWYQQKSGQPPKLLIYGASALASGVPSRFKGSGSGTEFTLTISDVQCDDAATYYCAGAITGSIDTDG

SEQ ID NO: 395 - protein, 130 aa, Oryctolagus cuniculus
METGLRWLLLVAVLKGVQCQSLEESGGDLVKPGASLTLTCTTSGFSFSSSYFICWVRQAPGKGLEWIACIYGGDGSTYYASWAKGRFTISKTSSTTVTLQMTSLTAADTATYFCAREWAYSQGYFGAFDL

SEQ ID NO: 396 - protein, 13 aa, Oryctolagus cuniculus
QASQSVYKNNQLS

SEQ ID NO: 397 - protein, 7 aa, Oryctolagus cuniculus
GASALAS

SEQ ID NO: 398 - protein, 12 aa, Oryctolagus cuniculus
AGAITGSIDTDG

SEQ ID NO: 399 - protein, 6 aa, Oryctolagus cuniculus
SSYFIC

SEQ ID NO: 400 - protein, 17 aa, Oryctolagus cuniculus
CIYGGDGSTYYASWAKG

SEQ ID NO: 401 - protein, 14 aa, Oryctolagus cuniculus
EWAYSQGYFGAFDL

SEQ ID NO: 402 - DNA, 372 bp, Oryctolagus cuniculus
atggacacgagggcccccactcagctgctggggctcctgctgctctggctcccaggtgtcacatttgccatcgaaatgacccagagtccattctccgtgtctgcagctgtgggaggcacagtcagcatcagttgccaggccagtcagagtgtttataagaacaaccaattatcctggtatcagcagaaatcagggcagcctcccaagctcctgatctatggtgcatcggctctggcatctggggtcccatcgcggttcaaaggcagtggatctgggacagagttcactctcaccatcagcgacgtgcagtgtgacgatgctgccacttactactgtgcaggcgctattactggtagtattgatacggatggt

SEQ ID NO: 403 - DNA, 390 bp, Oryctolagus cuniculus
atggagactgggctgcgctggcttctcctggtcgctgtgctcaaaggtgtccagtgtcagtcgttggaggagtccgggggagacctggtcaagcctggggcatccctgacactcacctgcacaacttctggattctccttcagtagcagctacttcatttgctgggtccgccaggctccagggaaggggctggagtggatcgcatgcatttatggtggtgatggcagcacatactacgcgagctgggcgaaaggccgattcaccatctccaaaacctcgtcgaccacggtgacgctgcaaatgaccagtctgacagccgcggacacggccacctatttctgtgcgagagaatgggcatatagtcaaggttattttggtgcttttgatctc

SEQ ID NO: 404 - DNA, 39 bp, Oryctolagus cuniculus
caggccagtcagagtgtttataagaacaaccaattatcc

SEQ ID NO: 405 - DNA, 21 bp, Oryctolagus cuniculus
ggtgcatcggctctggcatct

SEQ ID NO: 406 - DNA, 36 bp, Oryctolagus cuniculus
gcaggcgctattactggtagtattgatacggatggt

SEQ ID NO: 407 - DNA, 18 bp, Oryctolagus cuniculus
agcagctacttcatttgc

SEQ ID NO: 408 - DNA, 51 bp, Oryctolagus cuniculus
tgcatttatggtggtgatggcagcacatactacgcgagctgggcgaaaggc

SEQ ID NO: 409 - DNA, 42 bp, Oryctolagus cuniculus
gaatgggcatatagtcaaggttattttggtgcttttgatctc

SEQ ID NO: 410 - protein, 124 aa, Oryctolagus cuniculus
MDTRAPTQLLGLLLLWLPGARCDVVMTQTPASVEAAVGGTVTIKCQASEDISSYLAWYQQKPGQPPKLLIYAASNLESGVSSRFKGSGSGTEYTLTISDLECADAATYYCQCTYGTISISDGNA

SEQ ID NO: 411 - protein, 124 aa, Oryctolagus cuniculus
METGLRWLLLVAVLKGVQCQSVEESGGRLVTPGTPLTLTCTVSGFSLSSYFMTWVRQAPGEGLEYIGFINPGGSAYYASWVKGRFTISKSSTTVDLKITSPTTEDTATYFCARVLIVSYGAFTI

SEQ ID NO: 412 - protein, 11 aa, Oryctolagus cuniculus
QASEDISSYLA

SEQ ID NO: 413 - protein, 7 aa, Oryctolagus cuniculus
AASNLES

SEQ ID NO: 414 - protein, 14 aa, Oryctolagus cuniculus
QCTYGTISISDGNA

SEQ ID NO: 415 - protein, 5 aa, Oryctolagus cuniculus
SYFMT

SEQ ID NO: 416 - protein, 16 aa, Oryctolagus cuniculus
FINPGGSAYYASWVKG

SEQ ID NO: 417 - protein, 11 aa, Oryctolagus cuniculus
VLIVSYGAFTI

SEQ ID NO: 418 - DNA, 372 bp, Oryctolagus cuniculus
atggacacgagggcccccactcagctgctggggctcctgctgctctggctcccaggtgccagatgtgatgttgtgatgacccagactccagcctccgtggaggcagctgtgggaggcacagtcaccatcaagtgccaggccagtgaggatattagtagctacttagcctggtatcagcagaaaccagggcagcctcccaagctcctgatctatgctgcatccaatctggaatctggggtctcatcgcgattcaaaggcagtggatctgggacagagtacactctcaccatcagcgacctggagtgtgccgatgctgccacctattactgtcaatgtacttatggtactatttctattagtgatggtaatgct

SEQ ID NO: 419 - DNA, 372 bp, Oryctolagus cuniculus
atggagactgggctgcgctggcttctcctggtcgctgtgctcaaaggtgtccaatgtcagtcggtggaggagtccgggggtcgcctggtcacgcctgggacacccctgacactcacctgcacagtctctggattctccctcagtagctacttcatgacctgggtccgccaggctccaggggaggggctggaatacatcggattcattaatcctggtggtagcgcttactacgcgagctgggtgaaaggccgattcaccatctccaagtcctcgaccacggtagatctgaaaatcaccagtccgacaaccgaggacacggccacctatttctgtgccagggttctgattgtttcttatggagcctttaccatc

SEQ ID NO: 420 - DNA, 33 bp, Oryctolagus cuniculus
caggccagtgaggatattagtagctacttagcc

SEQ ID NO: 421 - DNA, 21 bp, Oryctolagus cuniculus
gctgcatccaatctggaatct

SEQ ID NO: 422 - DNA, 42 bp, Oryctolagus cuniculus
caatgtacttatggtactatttctattagtgatggtaatgct

SEQ ID NO: 423 - DNA, 15 bp, Oryctolagus cuniculus
agctacttcatgacc

SEQ ID NO: 424 - DNA, 48 bp, Oryctolagus cuniculus
ttcattaatcctggtggtagcgcttactacgcgagctgggtgaaaggc

SEQ ID NO: 425 - DNA, 33 bp, Oryctolagus cuniculus
gttctgattgtttcttatggagcctttaccatc

SEQ ID NO: 426 - protein, 124 aa, Oryctolagus cuniculus
MDTRAPTQLLGLLLLWLPGARCDVVMTQTPASVSAAVGGTVTIKCQASEDIESYLAWYQQKPGQPPKLLIYGASNLESGVSSRFKGSGSGTEFTLTISDLECADAATYYCQCTYGIISISDGNA

SEQ ID NO: 427 - protein, 124 aa, Oryctolagus cuniculus
METGLRWLLLVAVLKGVQCQSVEESGGRLVTPGTPLTLTCTVSGFSLSSYFMTWVRQAPGEGLEYIGFMNTGDNAYYASWAKGRFTISKTSTTVDLKITSPTTEDTATYFCARVLVVAYGAFNI

SEQ ID NO: 428 - protein, 11 aa, Oryctolagus cuniculus
QASEDIESYLA

SEQ ID NO: 429 - protein, 7 aa, Oryctolagus cuniculus
GASNLES

SEQ ID NO: 430 - protein, 14 aa, Oryctolagus cuniculus
QCTYGIISISDGNA

SEQ ID NO: 431 - protein, 5 aa, Oryctolagus cuniculus
SYFMT

SEQ ID NO: 432 - protein, 16 aa, Oryctolagus cuniculus
FMNTGDNAYYASWAKG

SEQ ID NO: 433 - protein, 11 aa, Oryctolagus cuniculus
VLVVAYGAFNI

SEQ ID NO: 434 - DNA, 372 bp, Oryctolagus cuniculus
atggacacgagggcccccactcagctgctggggctcctgctgctctggctcccaggtgccagatgtgatgttgtgatgacccagactccagcctccgtgtctgcagctgtgggaggcacagtcaccatcaagtgccaggccagtgaggacattgaaagctatctagcctggtatcagcagaaaccagggcagcctcccaagctcctgatctatggtgcatccaatctggaatctggggtctcatcgcggttcaaaggcagtggatctgggacagagttcactctcaccatcagcgacctggagtgtgccgatgctgccacttactattgtcaatgcacttatggtattattagtattagtgatggtaatgct

SEQ ID NO: 435 - DNA, 372 bp, Oryctolagus cuniculus
atggagactgggctgcgctggcttctcctggtcgctgtgctcaaaggtgtccagtgtcagtcggtggaggagtccgggggtcgcctggtcacgcctgggacacccctgacactcacctgcacagtgtctggattctccctcagtagctacttcatgacctgggtccgccaggctccaggggaggggctggaatacatcggattcatgaatactggtgataacgcatactacgcgagctgggcgaaaggccgattcaccatctccaaaacctcgaccacggtggatctgaaaatcaccagtccgacaaccgaggacacggccacctatttctgtgccagggttcttgttgttgcttatggagcctttaacatc

SEQ ID NO: 436 - DNA, 33 bp, Oryctolagus cuniculus
caggccagtgaggacattgaaagctatctagcc

SEQ ID NO: 437 - DNA, 21 bp, Oryctolagus cuniculus
ggtgcatccaatctggaatct

SEQ ID NO: 438 - DNA, 42 bp, Oryctolagus cuniculus
caatgcacttatggtattattagtattagtgatggtaatgct

SEQ ID NO: 439 - DNA, 15 bp, Oryctolagus cuniculus
agctacttcatgacc

SEQ ID NO: 440 - DNA, 48 bp, Oryctolagus cuniculus
ttcatgaatactggtgataacgcatactacgcgagctgggcgaaaggc

SEQ ID NO: 441 - DNA, 33 bp, Oryctolagus cuniculus
gttcttgttgttgcttatggagcctttaacatc

SEQ ID NO: 442 - protein, 124 aa, Oryctolagus cuniculus
MDTRAPTQLLGLLLLWLPGATFAAVLTQTPSPVSEPVGGTVSISCQSSKSVMNNNYLAWYQQKPGQPPKLLIYGASNLASGVPSRFSGSGSGTQFTLTISDVQCDDAATYYCQGGYTGYSDHGT

SEQ ID NO: 443 - protein, 127 aa, Oryctolagus cuniculus
METGLRWLLLVAVLKGVQCQSVEESGGRLVKPDETLTLTCTVSGIDLSSYPMNWVRQAPGKGLEWIGFINTGGTIVYASWAKGRFTISKTSTTVDLKMTSPTTEDTATYFCARGSYVSSGYAYYFNV

SEQ ID NO: 444 - protein, 13 aa, Oryctolagus cuniculus
QSSKSVMNNNYLA

SEQ ID NO: 445 - protein, 7 aa, Oryctolagus cuniculus
GASNLAS

SEQ ID NO: 446 - protein, 12 aa, Oryctolagus cuniculus
QGGYTGYSDHGT

SEQ ID NO: 447 - protein, 5 aa, Oryctolagus cuniculus
SYPMN

SEQ ID NO: 448 - protein, 16 aa, Oryctolagus cuniculus
FINTGGTIVYASWAKG

SEQ ID NO: 449 - protein, 14 aa, Oryctolagus cuniculus
GSYVSSGYAYYFNV

SEQ ID NO: 450 - DNA, 372 bp, Oryctolagus cuniculus
atggacacgagggcccccactcagctgctggggctcctgctgctctggctcccaggtgccacatttgccgccgtgctgacccagactccatctcccgtgtctgaacctgtgggaggcacagtcagcatcagttgccagtccagtaagagtgttatgaataacaactacttagcctggtatcagcagaaaccagggcagcctcccaagctcctgatctatggtgcatccaatctggcatctggggtcccatcacggttcagcggcagtggatctgggacacagttcactctcaccatcagcgacgtgcagtgtgacgatgctgccacttactactgtcaaggcggttatactggttatagtgatcatgggact

SEQ ID NO: 451 - DNA, 381 bp, Oryctolagus cuniculus
atggagactgggctgcgctggcttctcctggtcgctgtgctcaaaggtgtccagtgtcagtcggtggaggagtccgggggtcgcctggtcaagcctgacgaaaccctgacactcacctgcacagtctctggaatcgacctcagtagctatccaatgaactgggtccgccaggctccagggaaggggctggaatggatcggattcattaatactggtggtaccatagtctacgcgagctgggcaaaaggccgattcaccatctccaaaacctcgaccacggtggatctgaaaatgaccagtccgacaaccgaggacacggccacctatttctgtgccagaggcagttatgtttcatctggttatgcctactattttaatgtc

SEQ ID NO: 452 - DNA, 39 bp, Oryctolagus cuniculus
cagtccagtaagagtgttatgaataacaactacttagcc

SEQ ID NO: 453 - DNA, 21 bp, Oryctolagus cuniculus
ggtgcatccaatctggcatct

SEQ ID NO: 454 - DNA, 36 bp, Oryctolagus cuniculus
caaggcggttatactggttatagtgatcatgggact

SEQ ID NO: 455 - DNA, 15 bp, Oryctolagus cuniculus
agctatccaatgaac

SEQ ID NO: 456 - DNA, 48 bp, Oryctolagus cuniculus
ttcattaatactggtggtaccatagtctacgcgagctgggcaaaaggc

SEQ ID NO: 457 - DNA, 42 bp, Oryctolagus cuniculus
ggcagttatgtttcatctggttatgcctactattttaatgtc

SEQ ID NO: 458 - protein, 121 aa, Oryctolagus cuniculus
MDTRAPTQLLGLLLLWLPGATFAAVLTQTPSPVSAAVGGTVSISCQSSQSVYNNNWLSWFQQKPGQPPKLLIYKASTLASGVPSRFKGSGSGTQFTLTISDVQCDDVATYYCAGGYLDSVI

SEQ ID NO: 459 - protein, 126 aa, Oryctolagus cuniculus
METGLRWLLLVAVLKGVQCQSVEESGGRLVTPGTPLTLTCTVSGFSLSTYSINWVRQAPGKGLEWIGIIANSGTTFYANWAKGRFTVSKTSTTVDLKITSPTTEDTATYFCARESGMYNEYGKFNI

SEQ ID NO: 460 - protein, 13 aa, Oryctolagus cuniculus
QSSQSVYNNNWLS

SEQ ID NO: 461 - protein, 7 aa, Oryctolagus cuniculus
KASTLAS

SEQ ID NO: 462 - protein, 9 aa, Oryctolagus cuniculus
AGGYLDSVI

SEQ ID NO: 463 - protein, 5 aa, Oryctolagus cuniculus
TYSIN

SEQ ID NO: 464 - protein, 16 aa, Oryctolagus cuniculus
IIANSGTTFYANWAKG

SEQ ID NO: 465 - protein, 13 aa, Oryctolagus cuniculus
ESGMYNEYGKFNI

SEQ ID NO: 466 - DNA, 363 bp, Oryctolagus cuniculus
atggacacgagggcccccactcagctgctggggctcctgctgctctggctcccaggtgccacatttgccgccgtgctgacccagactccatctcccgtgtctgcagctgtgggaggcacagtcagcatcagttgccagtccagtcagagtgtttataataacaactggttatcctggtttcagcagaaaccagggcagcctcccaagctcctgatctacaaggcatccactctggcatctggggtcccatcgcggttcaaaggcagtggatctgggacacagttcactctcaccatcagcgacgtgcagtgtgacgatgttgccacttactactgtgcgggcggttatcttgatagtgttatt

SEQ ID NO: 467 - DNA, 378 bp, Oryctolagus cuniculus
atggagactgggctgcgctggcttctcctggtcgctgtgctcaaaggtgtccagtgtcagtcggtggaggagtccgggggtcgcctggtcacgcctgggacacccctgacactcacctgcacagtctctggattctccctcagtacctattcaataaactgggtccgccaggctccagggaagggcctggaatggatcggaatcattgctaatagtggtaccacattctacgcgaactgggcgaaaggccgattcaccgtctccaaaacctcgaccacggtggatctgaaaatcaccagtccgacaaccgaggacacggccacctatttctgtgccagagagagtggaatgtacaatgaatatggtaaatttaacatc

SEQ ID NO: 468 - DNA, 39 bp, Oryctolagus cuniculus
cagtccagtcagagtgtttataataacaactggttatcc

SEQ ID NO: 469 - DNA, 21 bp, Oryctolagus cuniculus
aaggcatccactctggcatct

SEQ ID NO: 470 - DNA, 27 bp, Oryctolagus cuniculus
gcgggcggttatcttgatagtgttatt

SEQ ID NO: 471 - DNA, 15 bp, Oryctolagus cuniculus
acctattcaataaac

SEQ ID NO: 472 - DNA, 48 bp, Oryctolagus cuniculus
atcattgctaatagtggtaccacattctacgcgaactgggcgaaaggc

SEQ ID NO: 473 - DNA, 39 bp, Oryctolagus cuniculus
gagagtggaatgtacaatgaatatggtaaatttaacatc

SEQ ID NO: 474 - protein, 122 aa, Oryctolagus cuniculus
MDTRAPTQLLGLLLLWLPGARCASDMTQTPSSVSAAVGGTVTINCQASENIYSFLAWYQQKPGQPPKLLIFKASTLASGVSSRFKGSGSGTQFTLTISDLECDDAATYYCQQGATVYDIDNN

SEQ ID NO: 475 - protein, 128 aa, Oryctolagus cuniculus
METGLRWLLLVAVLKGVQCQSLEESGGRLVTPGTPLTLTCTVSGIDLSAYAMIWVRQAPGEGLEWITIIYPNGITYYANWAKGRFTVSKTSTAMDLKITSPTTEDTATYFCARDAESSKNAYWGYFNV

SEQ ID NO: 476 - protein, 11 aa, Oryctolagus cuniculus
QASENIYSFLA

SEQ ID NO: 477 - protein, 7 aa, Oryctolagus cuniculus
KASTLAS

SEQ ID NO: 478 - protein, 12 aa, Oryctolagus cuniculus
QQGATVYDIDNN

SEQ ID NO: 479 - protein, 5 aa, Oryctolagus cuniculus
AYAMI

SEQ ID NO: 480 - protein, 16 aa, Oryctolagus cuniculus
IIYPNGITYYANWAKG

SEQ ID NO: 481 - protein, 15 aa, Oryctolagus cuniculus
DAESSKNAYWGYFNV

SEQ ID NO: 482 - DNA, 366 bp, Oryctolagus cuniculus
atggacacgagggcccccactcagctgctggggctcctgctgctctggctcccaggtgccagatgtgcctctgatatgacccagactccatcctccgtgtctgcagctgtgggaggcacagtcaccatcaattgccaggccagtgagaacatttatagctttttggcctggtatcagcagaaaccagggcagcctcccaagctcctgatcttcaaggcttccactctggcatctggggtctcatcgcggttcaaaggcagtggatctgggacacagttcactctcaccatcagcgacctggagtgtgacgatgctgccacttactactgtcaacagggtgctactgtgtatgatattgataataat

SEQ ID NO: 483 - DNA, 384 bp, Oryctolagus cuniculus
atggagactgggctgcgctggcttctcctggtcgctgtgctcaaaggtgtccagtgtcagtcgctggaggagtccgggggtcgcctggtcacgcctgggacacccctgacactcacctgcacagtttctggaatcgacctcagtgcctatgcaatgatctgggtccgccaggctccaggggaggggctggaatggatcacaatcatttatcctaatggtatcacatactacgcgaactgggcgaaaggccgattcaccgtctccaaaacctcgaccgcgatggatctgaaaatcaccagtccgacaaccgaggacacggccacctatttctgtgccagagatgcagaaagtagtaagaatgcttattggggctactttaacgtc

SEQ ID NO: 484 - DNA, 33 bp, Oryctolagus cuniculus
caggccagtgagaacatttatagctttttggcc

SEQ ID NO: 485 - DNA, 21 bp, Oryctolagus cuniculus
aaggcttccactctggcatct

SEQ ID NO: 486 - DNA, 36 bp, Oryctolagus cuniculus
caacagggtgctactgtgtatgatattgataataat

SEQ ID NO: 487 - DNA, 15 bp, Oryctolagus cuniculus
gcctatgcaatgatc

SEQ ID NO: 488 - DNA, 48 bp, Oryctolagus cuniculus
atcatttatcctaatggtatcacatactacgcgaactgggcgaaaggc

SEQ ID NO: 489 - DNA, 45 bp, Oryctolagus cuniculus
gatgcagaaagtagtaagaatgcttattggggctactttaacgtc

SEQ ID NO: 490 - protein, 122 aa, Oryctolagus cuniculus
MDTRAPTQLLGLLLLWLPGARCASDMTQTPSSVSAAVGGTVTINCQASENIYSFLAWYQQKPGQPPKLLIFRASTLASGVSSRFKGSGSGTQFTLTISDLECDDAATYYCQQGATVYDIDNN

SEQ ID NO: 491 - protein, 128 aa, Oryctolagus cuniculus; identical to SEQ ID NO: 475

SEQ ID NO: 492 - protein, 11 aa, Oryctolagus cuniculus
QASENIYSFLA

SEQ ID NO: 493 - protein, 7 aa, Oryctolagus cuniculus
RASTLAS

SEQ ID NO: 494 - protein, 12 aa, Oryctolagus cuniculus
QQGATVYDIDNN

SEQ ID NO: 495 - protein, 5 aa, Oryctolagus cuniculus
AYAMI

SEQ ID NO: 496 - protein, 16 aa, Oryctolagus cuniculus
IIYPNGITYYANWAKG

SEQ ID NO: 497 - protein, 15 aa, Oryctolagus cuniculus
DAESSKNAYWGYFNV

SEQ ID NO: 498 - DNA, 366 bp, Oryctolagus cuniculus
atggacacgagggcccccactcagctgctggggctcctgctgctctggctcccaggtgccagatgtgcctctgatatgacccagactccatcctccgtgtctgcagctgtgggaggcacagtcaccatcaattgccaggccagtgagaacatttatagctttttggcctggtatcagcagaaaccagggcagcctcccaagctcctgatcttcagggcttccactctggcatctggggtctcatcgcggttcaaaggcagtggatctgggacacagttcactctcaccatcagcgacctggagtgtgacgatgctgccacttactactgtcaacagggtgctactgtgtatgatattgataataat

SEQ ID NO: 499 - DNA, 384 bp, Oryctolagus cuniculus; identical to SEQ ID NO: 483

SEQ ID NO: 500 - DNA, 33 bp, Oryctolagus cuniculus; identical to SEQ ID NO: 484

SEQ ID NO: 501 - DNA, 21 bp, Oryctolagus cuniculus
agggcttccactctggcatct

SEQ ID NO: 502 - DNA, 36 bp, Oryctolagus cuniculus; identical to SEQ ID NO: 486

SEQ ID NO: 503 - DNA, 15 bp, Oryctolagus cuniculus
gcctatgcaatgatc

SEQ ID NO: 504 - DNA, 48 bp, Oryctolagus cuniculus; identical to SEQ ID NO: 488

SEQ ID NO: 505 - DNA, 45 bp, Oryctolagus cuniculus; identical to SEQ ID NO: 489

SEQ ID NO: 506 - protein, 124 aa, Oryctolagus cuniculus
MDTRAPTQLLGLLLLWLPGATFAIEMTQTPSPVSAAVGGTVTINCQASESVFNNMLSWYQQKPGHSPKLLIYDASDLASGVPSRFKGSGSGTQFTLTISGVECDDAATYYCAGYKSDSNDGDNV

SEQ ID NO: 507 - protein, 123 aa, Oryctolagus cuniculus
METGLRWLLLVAVLKGVQCQSLEESGGRLVTPGTPLTLTCTVSGFSLNRNSITWVRQAPGEGLEWIGIITGSGRTYYANWAKGRFTISKTSTTVDLKMTSPTTEDTATYFCARGHPGLGSGNI

SEQ ID NO: 508 - protein, 12 aa, Oryctolagus cuniculus
QASESVFNNMLS

SEQ ID NO: 509 - protein, 7 aa, Oryctolagus cuniculus
DASDLAS

SEQ ID NO: 510 - protein, 13 aa, Oryctolagus cuniculus
AGYKSDSNDGDNV

SEQ ID NO: 511 - protein, 5 aa, Oryctolagus cuniculus
RNSIT

SEQ ID NO: 512 - protein, 16 aa, Oryctolagus cuniculus
IITGSGRTYYANWAKG

SEQ ID NO: 513 - protein, 10 aa, Oryctolagus cuniculus
GHPGLGSGNI

SEQ ID NO: 514 - DNA, 372 bp, Oryctolagus cuniculus
atggacacgagggcccccactcagctgctggggctcctgctgctctggctcccaggtgccacatttgccattgaaatgacccagactccatcccccgtgtctgccgctgtgggaggcacagtcaccatcaattgccaggccagtgagagtgtttttaataatatgttatcctggtatcagcagaaaccagggcactctcctaagctcctgatctatgatgcatccgatctggcatctggggtcccatcgcggttcaaaggcagtggatctgggacacagttcactctcaccatcagtggcgtggagtgtgacgatgctgccacttactattgtgcagggtataaaagtgatagtaatgatggcgataatgtt

SEQ ID NO: 515 - DNA, 369 bp, Oryctolagus cuniculus
atggagactgggctgcgctggcttctcctggtcgctgtgctcaaaggtgtccagtgtcagtcgctggaggagtccgggggtcgcctggtcacgcctgggacacccctgacactcacctgcacagtctctggattctccctcaacaggaattcaataacctgggtccgccaggctccaggggaggggctggaatggatcggaatcattactggtagtggtagaacgtactacgcgaactgggcaaaaggccgattcaccatctccaaaacctcgaccacggtggatctgaaaatgaccagtccgacaaccgaggacacggccacctatttctgtgccagaggccatcctggtcttggtagtggtaacatc

SEQ ID NO: 516 - DNA, 36 bp, Oryctolagus cuniculus
caggccagtgagagtgtttttaataatatgttatcc

SEQ ID NO: 517 - DNA, 21 bp, Oryctolagus cuniculus
gatgcatccgatctggcatct

SEQ ID NO: 518 - DNA, 39 bp, Oryctolagus cuniculus
gcagggtataaaagtgatagtaatgatggcgataatgtt

SEQ ID NO: 519 - DNA, 15 bp, Oryctolagus cuniculus
aggaattcaataacc

SEQ ID NO: 520 - DNA, 48 bp, Oryctolagus cuniculus
atcattactggtagtggtagaacgtactacgcgaactgggcaaaaggc

SEQ ID NO: 521 - DNA, 30 bp, Oryctolagus cuniculus
ggccatcctggtcttggtagtggtaacatc

SEQ ID NO: 522 - protein, 121 aa, Oryctolagus cuniculus
MDTRAPTQLLGLLLLWLPGATFAQVLTQTASSVSAAVGGTVTINCQSSQSVYNNYLSWYQQKPGQPPKLLIYTASSLASGVPSRFKGSGSGTQFTLTISEVQCDDAATYYCQGYYSGPIIT

SEQ ID NO: 523 - protein, 122 aa, Oryctolagus cuniculus
METGLRWLLLVAVLKGVQCQSLEESGGRLVTPGTPLTLTCTASGFSLNNYYIQWVRQAPGEGLEWIGIIYAGGSAYYATWANGRFTIAKTSSTTVDLKMTSLTTEDTATYFCARGTFDGYEL

SEQ ID NO: 524 - protein, 12 aa, Oryctolagus cuniculus
QSSQSVYNNYLS

SEQ ID NO: 525 - protein, 7 aa, Oryctolagus cuniculus
TASSLAS

SEQ ID NO: 526 - protein, 10 aa, Oryctolagus cuniculus
QGYYSGPIIT

SEQ ID NO: 527 - protein, 5 aa, Oryctolagus cuniculus
NYYIQ

SEQ ID NO: 528 - protein, 16 aa, Oryctolagus cuniculus
IIYAGGSAYYATWANG

SEQ ID NO: 529 - protein, 8 aa, Oryctolagus cuniculus
GTFDGYEL

SEQ ID NO: 530 - DNA, 363 bp, Oryctolagus cuniculus
atggacacgagggcccccactcagctgctggggctcctgctgctctggctcccaggtgccacatttgcgcaagtgctgacccagactgcatcgtccgtgtctgcagctgtgggaggcacagtcaccatcaattgccagtccagtcagagtgtttataataactacttatcctggtatcagcagaaaccagggcagcctcccaagctcctgatctatactgcatccagcctggcatctggggtcccatcgcggttcaaaggcagtggatctgggacacagttcactctcaccatcagcgaagtgcagtgtgacgatgctgccacttactactgtcaaggctattatagtggtcctataattact

SEQ ID NO: 531 - DNA, 366 bp, Oryctolagus cuniculus
atggagactgggctgcgctggcttctcctggtcgctgtgctcaaaggtgtccagtgtcagtcgctggaggagtccgggggtcgcctggtcacgcctgggacacccctgacactcacctgcacagcctctggattctccctcaataactactacatacaatgggtccgccaggctccaggggaggggctggaatggatcgggatcatttatgctggtggtagcgcatactacgcgacctgggcaaacggccgattcaccatcgccaaaacctcgtcgaccacggtggatctgaagatgaccagtctgacaaccgaggacacggccacctatttctgtgccagagggacatttgatggttatgagttg

SEQ ID NO: 532 - DNA, 36 bp, Oryctolagus cuniculus
cagtccagtcagagtgtttataataactacttatcc

SEQ ID NO: 533 - DNA, 21 bp, Oryctolagus cuniculus
actgcatccagcctggcatct

SEQ ID NO: 534 - DNA, 30 bp, Oryctolagus cuniculus
caaggctattatagtggtcctataattact

SEQ ID NO: 535 - DNA, 15 bp, Oryctolagus cuniculus
aactactacatacaa

SEQ ID NO: 536 - DNA, 48 bp, Oryctolagus cuniculus
atcatttatgctggtggtagcgcatactacgcgacctgggcaaacggc

SEQ ID NO: 537 - DNA, 24 bp, Oryctolagus cuniculus
gggacatttgatggttatgagttg

SEQ ID NO: 538 - protein, 122 aa, Oryctolagus cuniculus
MDTRAPTQLLGLLLLWLPGATFAQVLTQTPSPVSVPVGDTVTISCQSSESVYSNNLLSWYQQKPGQPPKLLIYRASNLASGVPSRFKGSGSGTQFTLTISGAQCDDAATYYCQGYYSGVINS

SEQ ID NO: 539 - protein, 124 aa, Oryctolagus cuniculus
METGLRWLLLVAVLKGVQCQSVEESGGRLVTPGTPLTLTCTVSGFSLSSYFMSWVRQAPGEGLEYIGFINPGGSAYYASWASGRLTISKTSTTVDLKITSPTTEDTATYFCARILIVSYGAFTI

SEQ ID NO: 540 - protein, 13 aa, Oryctolagus cuniculus
QSSESVYSNNLLS

SEQ ID NO: 541 - protein, 7 aa, Oryctolagus cuniculus
RASNLAS

SEQ ID NO: 542 - protein, 10 aa, Oryctolagus cuniculus
QGYYSGVINS

SEQ ID NO: 543 - protein, 5 aa, Oryctolagus cuniculus
SYFMS

SEQ ID NO: 544 - protein, 16 aa, Oryctolagus cuniculus
FINPGGSAYYASWASG

SEQ ID NO: 545 - protein, 11 aa, Oryctolagus cuniculus
ILIVSYGAFTI

SEQ ID NO: 546 - DNA, 366 bp, Oryctolagus cuniculus
atggacacgagggcccccactcagctgctggggctcctgctgctctggctcccaggtgccacatttgcccaagtgctgacccagactccatcccctgtgtctgtccctgtgggagacacagtcaccatcagttgccagtccagtgagagcgtttatagtaataacctcttatcctggtatcagcagaaaccagggcagcctcccaagctcctgatctacagggcatccaatctggcatctggtgtcccatcgcggttcaaaggcagtggatctgggacacagttcactctcaccatcagcggcgcacagtgtgacgatgctgccacttactactgtcaaggctattatagtggtgtcattaatagt

SEQ ID NO: 547 - DNA, 372 bp, Oryctolagus cuniculus
atggagactgggctgcgctggcttctcctggtcgctgtgctcaaaggtgtccagtgtcagtcggtggaggagtccgggggtcgcctggtcacgcctgggacacccctgacactcacctgcacagtgtctggattctccctcagtagctacttcatgagctgggtccgccaggctccaggggaggggctggaatacatcggattcattaatcctggtggtagcgcatactacgcgagctgggcgagtggccgactcaccatctccaaaacctcgaccacggtagatctgaaaatcaccagtccgacaaccgaggacacggccacctatttctgtgccaggattcttattgtttcttatggagcctttaccatc

SEQ ID NO: 548 - DNA, 39 bp, Oryctolagus cuniculus
cagtccagtgagagcgtttatagtaataacctcttatcc

SEQ ID NO: 549 - DNA, 21 bp, Oryctolagus cuniculus
agggcatccaatctggcatct

SEQ ID NO: 550 - DNA, 30 bp, Oryctolagus cuniculus
caaggctattatagtggtgtcattaatagt

SEQ ID NO: 551 - DNA, 15 bp, Oryctolagus cuniculus
agctacttcatgagc

SEQ ID NO: 552 - DNA, 48 bp, Oryctolagus cuniculus
ttcattaatcctggtggtagcgcatactacgcgagctgggcgagtggc

SEQ ID NO: 553 - DNA, 33 bp, Oryctolagus cuniculus
attcttattgtttcttatggagcctttaccatc

SEQ ID NO: 554 - protein, 122 aa, Oryctolagus cuniculus
MDTRAPTQLLGLLLLWLPGARCAYDMTQTPASVEVAVGGTVTIKCQATESIGNELSWYQQKPGQAPKLLIYSASTLASGVPSRFKGSGSGTQFTLTITGVECDDAATYYCQQGYSSANIDNA

SEQ ID NO: 555 - protein, 128 aa, Oryctolagus cuniculus
METGLRWLLLVAVLKGVQCQSLEESGGRLVTPGTPLTLTCTVSGFSLSKYYMSWVRQAPEKGLKYIGYIDSTTVNTYYATWARGRFTISKTSTTVDLKITSPTSEDTATYFCARGSTYFTDGGHRLDL

SEQ ID NO: 556 - protein, 11 aa, Oryctolagus cuniculus
QATESIGNELS

SEQ ID NO: 557 - protein, 7 aa, Oryctolagus cuniculus
SASTLAS

SEQ ID NO: 558 - protein, 12 aa, Oryctolagus cuniculus
QQGYSSANIDNA

SEQ ID NO: 559 - protein, 5 aa, Oryctolagus cuniculus
KYYMS

SEQ ID NO: 560 - protein, 17 aa, Oryctolagus cuniculus
YIDSTTVNTYYATWARG

SEQ ID NO: 561 - protein, 14 aa, Oryctolagus cuniculus
GSTYFTDGGHRLDL

SEQ ID NO: 562 - DNA, 366 bp, Oryctolagus cuniculus
atggacacgagggcccccactcagctgctggggctcctgctgctctggctcccaggtgccagatgtgcctatgatatgacccagactccagcctctgtggaggtagctgtgggaggcacagtcaccatcaagtgccaggccactgagagcattggcaatgagttatcctggtatcagcagaaaccagggcaggctcccaagctcctgatctattctgcatccactctggcatctggggtcccatcgcggttcaaaggcagtggatctgggacacagttcactctcaccatcaccggcgtggagtgtgatgatgctgccacttactactgtcaacagggttatagtagtgctaatattgataatgct

SEQ ID NO: 563 - DNA, 384 bp, Oryctolagus cuniculus
atggagactgggctgcgctggcttctcctggtcgctgtgctcaaaggtgtccagtgtcagtcgctggaggagtccgggggtcgcctggtcacgcctgggacacccctgacactcacctgcaccgtctctggattctccctcagtaagtactacatgagctgggtccgccaggctccagagaaggggctgaaatacatcggatacattgatagtactactgttaatacatactacgcgacctgggcgagaggccgattcaccatctccaaaacctcgaccacggtggatctgaagatcaccagtccgacaagtgaggacacggccacctatttctgtgccagaggaagtacttattttactgatggaggccatcggttggatctc

SEQ ID NO: 564 - DNA, 33 bp, Oryctolagus cuniculus
caggccactgagagcattggcaatgagttatcc

SEQ ID NO: 565 - DNA, 21 bp, Oryctolagus cuniculus
tctgcatccactctggcatct

SEQ ID NO: 566 - DNA, 36 bp, Oryctolagus cuniculus
caacagggttatagtagtgctaatattgataatgct

SEQ ID NO: 567 - DNA, 15 bp, Oryctolagus cuniculus
aagtactacatgagc

SEQ ID NO: 568 - DNA, 51 bp, Oryctolagus cuniculus
tacattgatagtactactgttaatacatactacgcgacctgggcgagaggc

SEQ ID NO: 569 - DNA, 42 bp, Oryctolagus cuniculus
ggaagtacttattttactgatggaggccatcggttggatctc

SEQ ID NO: 570 - protein, 122 aa, Oryctolagus cuniculus; identical to SEQ ID NO: 554

SEQ ID NO: 571 - protein, 124 aa, Oryctolagus cuniculus
METGLRWLLLVAVLKGVQCQSLEESGGRLVTPGTPLTLTCTVSGFSLSTYNMGWVRQAPGKGLEWIGSITIDGRTYYASWAKGRFTVSKSSTTVDLKMTSLTTGDTATYFCARILIVSYGAFTI

SEQ ID NO: 572 - protein, 11 aa, Oryctolagus cuniculus
QATESIGNELS

SEQ ID NO: 573 - protein, 7 aa, Oryctolagus cuniculus
SASTLAS

SEQ ID NO: 574 - protein, 12 aa, Oryctolagus cuniculus
QQGYSSANIDNA

SEQ ID NO: 575 - protein, 5 aa, Oryctolagus cuniculus
TYNMG

SEQ ID NO: 576 - protein, 16 aa, Oryctolagus cuniculus
SITIDGRTYYASWAKG

SEQ ID NO: 577 - protein, 11 aa, Oryctolagus cuniculus
ILIVSYGAFTI

SEQ ID NO: 578 - DNA, 366 bp, Oryctolagus cuniculus; identical to SEQ ID NO: 562

SEQ ID NO: 579 - DNA, 372 bp, Oryctolagus cuniculus
atggagactgggctgcgctggcttctcctggtcgctgtgctcaaaggtgtccagtgtcagtcgctggaggagtccgggggtcgcctggtaacgcctgggacacccctgacactcacctgcacagtctctggattctccctcagtacctacaacatgggctgggtccgccaggctccagggaaggggctggaatggatcggaagtattactattgatggtcgcacatactacgcgagctgggcgaaaggccgattcaccgtctccaaaagctcgaccacggtggatctgaaaatgaccagtctgacaaccggggacacggccacctatttctgtgccaggattcttattgtttcttatggggcctttaccatc

SEQ ID NO: 580 - DNA, 33 bp, Oryctolagus cuniculus; identical to SEQ ID NO: 564

SEQ ID NO: 581 - DNA, 21 bp, Oryctolagus cuniculus
tctgcatccactctggcatct

SEQ ID NO: 582 - DNA, 36 bp, Oryctolagus cuniculus; identical to SEQ ID NO: 566

SEQ ID NO: 583 - DNA, 15 bp, Oryctolagus cuniculus
acctacaacatgggc

SEQ ID NO: 584 - DNA, 48 bp, Oryctolagus cuniculus
agtattactattgatggtcgcacatactacgcgagctgggcgaaaggc

SEQ ID NO: 585 - DNA, 33 bp, Oryctolagus cuniculus
attcttattgtttcttatggggcctttaccatc

SEQ ID NO: 586 - protein, 105 aa, Artificial Sequence [sequence omitted]

SEQ ID NO: 587 - DNA, 315 bp, Artificial Sequence
  Kappa constant domain
gtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 588 - protein, 330 aa, Artificial Sequence
  Gamma-1 constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 589 - DNA, 990 bp, Artificial Sequence
  Gamma-1 constant domain
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 590 - protein, 15 aa, Homo sapiens
VPPGEDSKDVAAPHR

SEQ ID NO: 591 - protein, 15 aa, Homo sapiens
GEDSKDVAAPHRQPL

SEQ ID NO: 592 - protein, 15 aa, Homo sapiens
SKDVAAPHRQPLTSS

SEQ ID NO: 593 - protein, 15 aa, Homo sapiens
VAAPHRQPLTSSERI

SEQ ID NO: 594 - protein, 15 aa, Homo sapiens
PHRQPLTSSERIDKQ

SEQ ID NO: 595 - protein, 15 aa, Homo sapiens
QPLTSSERIDKQIRY

SEQ ID NO: 596 - protein, 15 aa, Homo sapiens
TSSERIDKQIRYILD

SEQ ID NO: 597 - protein, 15 aa, Homo sapiens
ERIDKQIRYILDGIS

SEQ ID NO: 598 - protein, 15 aa, Homo sapiens
DKQIRYILDGISALR

SEQ ID NO: 599 - protein, 15 aa, Homo sapiens
IRYILDGISALRKET

SEQ ID NO: 600 - protein, 15 aa, Homo sapiens
ILDGISALRKETCNK

SEQ ID NO: 601 - protein, 15 aa, Homo sapiens
GISALRKETCNKSNM

SEQ ID NO: 602 - protein, 15 aa, Homo sapiens
ALRKETCNKSNMCES

SEQ ID NO: 603 - protein, 15 aa, Homo sapiens
KETCNKSNMCESSKE

SEQ ID NO: 604 - protein, 15 aa, Homo sapiens
CNKSNMCESSKEALA

SEQ ID NO: 605 - protein, 15 aa, Homo sapiens
SNMCESSKEALAENN

SEQ ID NO: 606 - protein, 15 aa, Homo sapiens
CESSKEALAENNLNL

SEQ ID NO: 607 - protein, 15 aa, Homo sapiens
SKEALAENNLNLPKM

SEQ ID NO: 608 - protein, 15 aa, Homo sapiens
ALAENNLNLPKMAEK

SEQ ID NO: 609 - protein, 15 aa, Homo sapiens
ENNLNLPKMAEKDGC

SEQ ID NO: 610 - protein, 15 aa, Homo sapiens
LNLPKMAEKDGCFQS

SEQ ID NO: 611 - protein, 15 aa, Homo sapiens
PKMAEKDGCFQSGFN

SEQ ID NO: 612 - protein, 15 aa, Homo sapiens
AEKDGCFQSGFNEET

SEQ ID NO: 613 - protein, 15 aa, Homo sapiens
DGCFQSGFNEETCLV

SEQ ID NO: 614 - protein, 15 aa, Homo sapiens
FQSGFNEETCLVKII

SEQ ID NO: 615 - protein, 15 aa, Homo sapiens
GFNEETCLVKIITGL

SEQ ID NO: 616 - protein, 15 aa, Homo sapiens
EETCLVKIITGLLEF

SEQ ID NO: 617 - protein, 15 aa, Homo sapiens
CLVKIITGLLEFEVY

SEQ ID NO: 618 - protein, 15 aa, Homo sapiens
KIITGLLEFEVYLEY

SEQ ID NO: 619 - protein, 15 aa, Homo sapiens
TGLLEFEVYLEYLQN

SEQ ID NO: 620 - protein, 15 aa, Homo sapiens
LEFEVYLEYLQNRFE

SEQ ID NO: 621 - protein, 15 aa, Homo sapiens
EVYLEYLQNRFESSE

SEQ ID NO: 622 - protein, 15 aa, Homo sapiens
LEYLQNRFESSEEQA

SEQ ID NO: 623 - protein, 15 aa, Homo sapiens
LQNRFESSEEQARAV

SEQ ID NO: 624 - protein, 15 aa, Homo sapiens
RFESSEEQARAVQMS

SEQ ID NO: 625 - protein, 15 aa, Homo sapiens
SSEEQARAVQMSTKV

SEQ ID NO: 626 - protein, 15 aa, Homo sapiens
EQARAVQMSTKVLIQ

SEQ ID NO: 627 - protein, 15 aa, Homo sapiens
RAVQMSTKVLIQFLQ

SEQ ID NO: 628 - protein, 15 aa, Homo sapiens
QMSTKVLIQFLQKKA

SEQ ID NO: 629 - protein, 15 aa, Homo sapiens
TKVLIQFLQKKAKNL

SEQ ID NO: 630 - protein, 15 aa, Homo sapiens
LIQFLQKKAKNLDAI

SEQ ID NO: 631 - protein, 15 aa, Homo sapiens
FLQKKAKNLDAITTP

SEQ ID NO: 632 - protein, 15 aa, Homo sapiens
KKAKNLDAITTPDPT

SEQ ID NO: 633 - protein, 15 aa, Homo sapiens
KNLDAITTPDPTTNA

SEQ ID NO: 634 - protein, 15 aa, Homo sapiens
DAITTPDPTTNASLL

SEQ ID NO: 635 - protein, 15 aa, Homo sapiens
TTPDPTTNASLLTKL

SEQ ID NO: 636 - protein, 15 aa, Homo sapiens
DPTTNASLLTKLQAQ

SEQ ID NO: 637 - protein, 15 aa, Homo sapiens
TNASLLTKLQAQNQW

SEQ ID NO: 638 - protein, 15 aa, Homo sapiens
SLLTKLQAQNQWLQD

SEQ ID NO: 639 - protein, 15 aa, Homo sapiens
TKLQAQNQWLQDMTT

SEQ ID NO: 640 - protein, 15 aa, Homo sapiens
QAQNQWLQDMTTHLI

SEQ ID NO: 641 - protein, 15 aa, Homo sapiens
NQWLQDMTTHLILRS

SEQ ID NO: 642 - protein, 15 aa, Homo sapiens
LQDMTTHLILRSFKE

SEQ ID NO: 643 - protein, 15 aa, Homo sapiens
MTTHLILRSFKEFLQ

SEQ ID NO: 644 - protein, 15 aa, Homo sapiens
HLILRSFKEFLQSSL

SEQ ID NO: 645 - protein, 15 aa, Homo sapiens
LRSFKEFLQSSLRAL

SEQ ID NO: 646 - protein, 15 aa, Homo sapiens
FKEFLQSSLRALRQM

SEQ ID NO: 647 - protein, 111 aa, Oryctolagus cuniculus
AYDMTQTPASVSAAVGGTVTIKCQASQSINNELSWYQQKPGQRPKLLIYRASTLASGVSSRFKGSGSGTEFTLTISDLECADAATYYCQQGYSLRNIDNAFGGGTEVVVKR

SEQ ID NO: 648 - protein, 88 aa, Homo sapiens
AIQMTQSPSSLSASVGDRVTITCRASQGIRNDLGWYQQKPGKAPKLLIYAASSLQSGVPSRFSGSGSGTDFTLTISSLQPEDFATYYC

SEQ ID NO: 649 - protein, 88 aa, Homo sapiens
DIQMTQSPSSLSASVGDRVTITCRASQGISNYLAWYQQKPGKVPKLLIYAASTLQSGVPSRFSGSGSGTDFTLTISSLQPEDVATYYC

SEQ ID NO: 650 - protein, 88 aa, Homo sapiens
DIQMTQSPSTLSASVGDRVTITCRASQSISSWLAWYQQKPGKAPKLLIYKASSLESGVPSRFSGSGSGTEFTLTISSLQPDDFATYYC

SEQ ID NO: 651 - protein, 111 aa, Artificial Sequence
  Humanized antibody
AIQMTQSPSSLSASVGDRVTITCQASQSINNELSWYQQKPGKAPKLLIYRASTLASGVPSRFSGSGSGTDFTLTISSLQPEDFATYYCQQGYSLRNIDNAFGGGTKVEIKR

SEQ ID NO: 652 - protein, 117 aa, Oryctolagus cuniculus
QSLEESGGRLVTPGTPLTLTCTASGFSLSNYYVTWVRQAPGKGLEWIGIIYGSDETAYATWAIGRFTISKTSTTVDLKMTSLTAADTATYFCARDDSSDWDAKFNLWGQGTLVTVSS

SEQ ID NO: 653 - protein, 97 aa, Homo sapiens
EVQLVESGGGLVQPGGSLRLSCAASGFTVSSNYMSWVRQAPGKGLEWVSVIYSGGSTYYADSVKGRFTISRDNSKNTLYLQMNSLRAEDTAVYYCAR

SEQ ID NO: 654 - protein, 97 aa, Homo sapiens
EVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCAR

SEQ ID NO: 655 - protein, 98 aa, Homo sapiens
EVQLLESGGGLVQPGGSLRLSCAASGFTFSSYAMSWVRQAPGKGLEWVSVIYSGGSSTYYADSVKGRFTISRDNSKNTLYLQMNSLRAEDTAVYYCAK

SEQ ID NO: 656 - protein, 120 aa, Artificial sequence
  Humanized antibody
EVQLVESGGGLVQPGGSLRLSCAASGFSLSNYYVTWVRQAPGKGLEWVGIIYGSDETAYATWAIGRFTISRDNSKNTLYLQMNSLRAEDTAVYYCARDDSSDWDAKFNLWGQGTLVTVSS

SEQ ID NO: 657 - protein, 120 aa, Artificial sequence
  Humanized antibody
EVQLVESGGGLVQPGGSLRLSCAASGFSLSNYYVTWVRQAPGKGLEWVGIIYGSDETAYATSAIGRFTISRDNSKNTLYLQMNSLRAEDTAVYYCARDDSSDWDAKFNLWGQGTLVTVSS

SEQ ID NO: 658 - protein, 166 aa, Oryctolagus cuniculus
METGLRWLLLVAVLKGVQCQSLEESGGRLVTPGTPLTLTCTASGFSLSNYYVTWVRQAPGKGLEWIGIIYGSDETAYATSAIGRFTISKTSTTVDLKMTSLTAADTATYFCARDDSSDWDAKFNLWGQGTLVTVSSASTKGPSVFPLAPSSKSTSGGTAALGCLVK

SEQ ID NO: 659 - protein, 16 aa, Oryctolagus cuniculus
IIYGSDETAYATSAIG

SEQ ID NO: 660 - protein, 122 aa, Oryctolagus cuniculus
MDTRAPTQLLGLLLLWLPGARCAYDMTQTPASVSAAVGGTVTIKCQASQSINNELSWYQQKPGQRPKLLIYRASTLASGVSSRFKGSGSGTEFTLTISDLECADAATYYCQQGYSLRNIDNA

SEQ ID NO: 661 - protein, 125 aa, Oryctolagus cuniculus
METGLRWLLLVAVLKGVQCQSLEESGGRLVTPGTPLTLTCTASGFSLSNYYVTWVRQAPGKGLEWIGIIYGSDETAYATWAIGRFTISKTSTTVDLKMTSLTAADTATYFCARDDSSDWDAKFNL

SEQ ID NO: 662 - DNA, 366 bp, Oryctolagus cuniculus
atggacacgagggcccccactcagctgctggggctcctgctgctctggctcccaggtgccagatgtgcctatgatatgacccagactccagcctcggtgtctgcagctgtgggaggcacagtcaccatcaagtgccaggccagtcagagcattaacaatgaattatcctggtatcagcagaaaccagggcagcgtcccaagctcctgatctatagggcatccactctggcatctggggtctcatcgcggttcaaaggcagtggatctgggacagagttcactctcaccatcagcgacctggagtgtgccgatgctgccacttactactgtcaacagggttatagtctgaggaatattgataatgct

SEQ ID NO: 663 - DNA, 375 bp, Oryctolagus cuniculus
atggagactgggctgcgctggcttctcctggtcgctgtgctcaaaggtgtccagtgtcagtcgctggaggagtccgggggtcgcctggtcacgcctgggacacccctgacactcacctgcacagcctctggattctccctcagtaactactacgtgacctgggtccgccaggctccagggaaggggctggaatggatcggaatcatttatggtagtgatgaaacggcctacgcgacctgggcgataggccgattcaccatctccaaaacctcgaccacggtggatctgaaaatgaccagtctgacagccgcggacacggccacctatttctgtgccagagatgatagtagtgactgggatgcaaaatttaacttg

SEQ ID NO: 664 - protein, 450 aa, Oryctolagus cuniculus
EVQLVESGGGLVQPGGSLRLSCAASGFSLSNYYVTWVRQAPGKGLEWVGIIYGSDETAYATWAIGRFTISRDNSKNTLYLQMNSLRAEDTAVYYCARDDSSDWDAKFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 665 - protein, 450 aa, Oryctolagus cuniculus
EVQLVESGGGLVQPGGSLRLSCAASGFSLSNYYVTWVRQAPGKGLEWVGIIYGSDETAYATSAIGRFTISRDNSKNTLYLQMNSLRAEDTAVYYCARDDSSDWDAKFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 666 - protein, 216 aa, Oryctolagus cuniculus
IQMTQSPSSLSASVGDRVTITCQASQSINNELSWYQQKPGKAPKLLIYRASTLASGVPSRFSGSGSGTDFTLTISSLQPDDFATYYCQQGYSLRNIDNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 667 - protein, 122 aa, Oryctolagus cuniculus
MDTRAPTQLLGLLLLWLPGARCAYDMTQTPASVEVAVGGTVTINCQASETIYSWLSWYQQKPGQPPKLLIYQASDLASGVPSRFSGSGAGTEYTLTISGVQCDDAATYYCQQGYSGSNVDNV

SEQ ID NO: 668 - protein, 126 aa, Oryctolagus cuniculus
METGLRWLLLVAVLKGVQCQEQLKESGGRLVTPGTPLTLTCTASGFSLNDHAMGWVRQAPGKGLEYIGFINSGGSARYASWAEGRFTISRTSTTVDLKMTSLTTEDTATYFCVRGGAVWSIHSFDP

SEQ ID NO: 669 - DNA, 366 bp, Oryctolagus cuniculus
atggacacgagggcccccactcagctgctggggctcctgctgctctggctcccaggtgccagatgtgcctatgatatgacccagactccagcctctgtggaggtagctgtgggaggcacagtcaccatcaattgccaggccagtgagaccatttacagttggttatcctggtatcagcagaagccagggcagcctcccaagctcctgatctaccaggcatccgatctggcatctggggtcccatcgcgattcagcggcagtggggctgggacagagtacactctcaccatcagcggcgtgcagtgtgacgatgctgccacttactactgtcaacagggttatagtggtagtaatgttgataatgtt

SEQ ID NO: 670 - DNA, 378 bp, Oryctolagus cuniculus
atggagactgggctgcgctggcttctcctggtcgctgtgctcaaaggtgtccagtgtcaggagcagctgaaggagtccgggggtcgcctggtcacgcctgggacacccctgacacttacctgcacagcctctggattctccctcaatgaccatgcaatgggctgggtccgccaggctccagggaaggggctggaatacatcggattcattaatagtggtggtagcgcacgctacgcgagctgggcagaaggccgattcaccatctccagaacctcgaccacggtggatctgaaaatgaccagtctgacaaccgaggacacggccacctatttctgtgtcagagggggtgctgtttggagtattcatagttttgatccc

SEQ ID NO: 671 - protein, 123 aa, Oryctolagus cuniculus
MDTRAPTQLLGLLLLWLPGATFAAVLTQTPSPVSAAVGGTVSISCQASQSVYDNNYLSWFQQKPGQPPKLLIYGASTLASGVPSRFVGSGSGTQFTLTITDVQCDDAATYYCAGVYDDDSDNA

SEQ ID NO: 672 - protein, 125 aa, Oryctolagus cuniculus
METGLRWLLLVAVLKGVQCQSLEESGGRLVTPGTPLTLTCTASGFSLSVYYMNWVRQAPGKGLEWIGFITMSDNINYASWAKGRFTISKTSTTVDLKMTSPTTEDTATYFCARSRGWGTMGRLDL

SEQ ID NO: 673 - DNA, 369 bp, Oryctolagus cuniculus
atggacacgagggcccccactcagctgctggggctcctgctgctctggctcccaggtgccacatttgccgccgtgctgacccagactccatctcccgtgtctgcagctgtgggaggcacagtcagcatcagttgccaggccagtcagagtgtttatgacaacaactacttatcctggtttcagcagaaaccagggcagcctcccaagctcctgatctatggtgcatccactctggcatctggggtcccatcgcggttcgtgggcagtggatctgggacacagttcactctcaccatcacagacgtgcagtgtgacgatgctgccacttactattgtgcaggcgtttatgatgatgatagtgataatgcc

SEQ ID NO: 674 - DNA, 375 bp, Oryctolagus cuniculus
atggagactgggctgcgctggcttctcctggtggctgtgctcaaaggtgtccagtgtcagtcgctggaggagtccgggggtcgcctggtcacccctgggacacccctgacactcacctgcacagcctctggattctccctcagtgtctactacatgaactgggtccgccaggctccagggaaggggctggaatggatcggattcattacaatgagtgataatataaattacgcgagctgggcgaaaggccgattcaccatctccaaaacctcgaccacggtggatctgaaaatgaccagtccgacaaccgaggacacggccacctatttctgtgccaggagtcgtggctggggtacaatgggtcggttggatctc

SEQ ID NO: 675 - protein, 123 aa, Oryctolagus cuniculus
MDTRAPTQLLGLLLLWLPGAICDPVLTQTPSPVSAPVGGTVSISCQASQSVYENNYLSWFQQKPGQPPKLLIYGASTLDSGVPSRFKGSGSGTQFTLTITDVQCDDAATYYCAGVYDDDSDDA

SEQ ID NO: 676 - protein, 126 aa, Oryctolagus cuniculus
METGLRWLLLVAVLKGVQCQEQLKESGGGLVTPGGTLTLTCTASGFSLNAYYMNWVRQAPGKGLEWIGFITLNNNVAYANWAKGRFTFSKTSTTVDLKMTSPTPEDTATYFCARSRGWGAMGRLDL

SEQ ID NO: 677 - DNA, 369 bp, Oryctolagus cuniculus
atggacacgagggcccccactcagctgctggggctcctgctgctctggctcccaggtgccatatgtgaccctgtgctgacccagactccatctcccgtatctgcacctgtgggaggcacagtcagcatcagttgccaggccagtcagagtgtttatgagaacaactatttatcctggtttcagcagaaaccagggcagcctcccaagctcctgatctatggtgcatccactctggattctggggtcccatcgcggttcaaaggcagtggatctgggacacagttcactctcaccattacagacgtgcagtgtgacgatgctgccacttactattgtgcaggcgtttatgatgatgatagtgatgatgcc

SEQ ID NO: 678 - DNA, 378 bp, Oryctolagus cuniculus
atggagactgggctgcgctggcttctcctggtggctgtgctcaaaggtgtccagtgtcaggagcagctgaaggagtccggaggaggcctggtaacgcctggaggaaccctgacactcacctgcacagcctctggattctccctcaatgcctactacatgaactgggtccgccaggctccagggaaggggctggaatggatcggattcattactctgaataataatgtagcttacgcgaactgggcgaaaggccgattcaccttctccaaaacctcgaccacggtggatctgaaaatgaccagtccgacacccgaggacacggccacctatttctgtgccaggagtcgtggctggggtgcaatgggtcggttggatctc

SEQ ID NO: 679 - protein, 122 aa, Oryctolagus cuniculus
MDTRAPTQLLGLLLLWLPGATFAQVLTQTPSPVSAAVGGTVTINCQASQSVDDNNWLGWYQQKRGQPPKYLIYSASTLASGVPSRFKGSGSGTQFTLTISDLECDDAATYYCAGGFSGNIFA

SEQ ID NO: 680 - protein, 122 aa, Oryctolagus cuniculus
METGLRWLLLVAVLKGVQCQSVEESGGRLVTPGTPLTLTCTVSGFSLSSYAMSWVRQAPGKGLEWIGIIGGFGTTYYATWAKGRFTISKTSTTVDLRITSPTTEDTATYFCARGGPGNGGDI

SEQ ID NO: 681 - DNA, 366 bp, Oryctolagus cuniculus
atggacacgagggcccccactcagctgctggggctcctgctgctctggctcccaggtgccacatttgcccaagtgctgacccagactccatcgcctgtgtctgcagctgtgggaggcacagtcaccatcaactgccaggccagtcagagtgttgatgataacaactggttaggctggtatcagcagaaacgagggcagcctcccaagtacctgatctattctgcatccactctggcatctggggtcccatcgcggttcaaaggcagtggatctgggacacagttcactctcaccatcagcgacctggagtgtgacgatgctgccacttactactgtgcaggcggttttagtggtaatatctttgct

SEQ ID NO: 682 - DNA, 366 bp, Oryctolagus cuniculus
atggagactgggctgcgctggcttctcctggtcgctgtgctcaaaggtgtccagtgtcagtcggtggaggagtccgggggtcgcctggtcacgcctgggacacccctgacactcacctgcacagtctctggcttctccctcagtagctatgcaatgagctgggtccgccaggctccaggaaaggggctggagtggatcggaatcattggtggttttggtaccacatactacgcgacctgggcgaaaggccgattcaccatctccaaaacctcgaccacggtggatctgagaatcaccagtccgacaaccgaggacacggccacctatttctgtgccagaggtggtcctggtaatggtggtgacatc

SEQ ID NO: 683 - protein, 122 aa, Oryctolagus cuniculus
MDTRAPTQLLGLLLLWLPGATFAAVLTQTPSPVSVPVGGTVTIKCQSSQSVYNNFLSWYQQKPGQPPKLLIYQASKLASGVPDRFSGSGSGTQFTLTISGVQCDDAATYYCLGGYDDDADNA

SEQ ID NO: 684 - protein, 128 aa, Oryctolagus cuniculus
METGLRWLLLVAVLKGVQCQSVEESGGRLVTPGTPLTLTCTVSGIDLSDYAMSWVRQAPGKGLEWIGIIYAGSGSTWYASWAKGRFTISKTSTTVDLKITSPTTEDTATYFCARDGYDDYGDFDRLDL

SEQ ID NO: 685 - DNA, 366 bp, Oryctolagus cuniculus
atggacacgagggcccccactcagctgctggggctcctgctgctctggctcccaggtgccacatttgcagccgtgctgacccagacaccatcgcccgtgtctgtacctgtgggaggcacagtcaccatcaagtgccagtccagtcagagtgtttataataatttcttatcgtggtatcagcagaaaccagggcagcctcccaagctcctgatctaccaggcatccaaactggcatctggggtcccagataggttcagcggcagtggatctgggacacagttcactctcaccatcagcggcgtgcagtgtgacgatgctgccacttactactgtctaggcggttatgatgatgatgctgataatgct

SEQ ID NO: 686 - DNA, 384 bp, Oryctolagus cuniculus
atggagactgggctgcgctggcttctcctggtcgctgtgctcaaaggtgtccagtgtcagtcggtggaggagtccgggggtcgcctggtcacgcctgggacacccctgacgctcacctgcacagtctctggaatcgacctcagtgactatgcaatgagctgggtccgccaggctccagggaaggggctggaatggatcggaatcatttatgctggtagtggtagcacatggtacgcgagctgggcgaaaggccgattcaccatctccaaaacctcgaccacggtggatctgaaaatcaccagtccgacaaccgaggacacggccacctatttctgtgccagagatggatacgatgactatggtgatttcgatcgattggatctc

SEQ ID NO: 687 - protein, 122 aa, Oryctolagus cuniculus
MDTRAPTQLLGLLLLWLPGARCAYDMTQTPASVSAAVGGTVTIKCQASQSINNELSWYQQKSGQRPKLLIYRASTLASGVSSRFKGSGSGTEFTLTISDLECADAATYYCQQGYSLRNIDNA

SEQ ID NO: 688 - protein, 125 aa, Oryctolagus cuniculus
METGLRWLLLVAVLSGVQCQSLEESGGRLVTPGTPLTLTCTASGFSLSNYYMTWVRQAPGKGLEWIGMIYGSDETAYANWAIGRFTISKTSTTVDLKMTSLTAADTATYFCARDDSSDWDAKFNL

SEQ ID NO: 689 - DNA, 366 bp, Oryctolagus cuniculus
atggacacgagggcccccactcagctgctggggctcctgctgctctggctcccaggtgccagatgtgcctatgatatgacccagactccagcctcggtgtctgcagctgtgggaggcacagtcaccatcaaatgccaggccagtcagagcattaacaatgaattatcctggtatcagcagaaatcagggcagcgtcccaagctcctgatctatagggcatccactctggcatctggggtctcatcgcggttcaaaggcagtggatctgggacagagttcactctcaccatcagcgacctggagtgtgccgatgctgccacttactactgtcaacagggttatagtctgaggaatattgataatgct

SEQ ID NO: 690 - DNA, 375 bp, Oryctolagus cuniculus
atggagactgggctgcgctggcttctcctggtcgctgtgctctcaggtgtccagtgtcagtcgctggaggagtccgggggtcgcctggtcacgcctgggacacccctgacactcacctgcacagcctctggattctccctcagtaactactacatgacctgggtccgccaggctccagggaaggggctggaatggatcggaatgatttatggtagtgatgaaacagcctacgcgaactgggcgataggccgattcaccatctccaaaacctcgaccacggtggatctgaaaatgaccagtctgacagccgcggacacggccacctatttctgtgccagagatgatagtagtgactgggatgcaaaatttaacttg

SEQ ID NO: 691 - protein, 450 aa, Oryctolagus cuniculus
EVQLVESGGGLVQPGGSLRLSCAASGFSLSNYYMTWVRQAPGKGLEWVGMIYGSDETAYANWAIGRFTISRDNSKNTLYLQMNSLRAEDTAVYYCARDDSSDWDAKFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 692 - protein, 450 aa, Oryctolagus cuniculus
EVQLVESGGGLVQPGGSLRLSCAASGFSLSNYYMTWVRQAPGKGLEWVGMIYGSDETAYANSAIGRFTISRDNSKNTLYLQMNSLRAEDTAVYYCARDDSSDWDAKFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 693 - protein, 217 aa, Oryctolagus cuniculus
DIQMTQSPSTLSASVGDRVTITCQASQSINNELSWYQQKPGKAPKLLIYRASTLASGVPSRFSGSGSGTEFTLTISSLQPDDFATYYCQQGYSLRNIDNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 694 - DNA, 33 bp, Oryctolagus cuniculus
caggccagtcagagcattaacaatgagttatcc

SEQ ID NO: 695 - DNA, 36 bp, Oryctolagus cuniculus
caacagggttatagtctgaggaacattgataatgct

SEQ ID NO: 696 - DNA, 48 bp, Oryctolagus cuniculus
atcatctatggtagtgatgaaaccgcctacgctacctccgctataggc

SEQ ID NO: 697 - DNA, 36 bp, Oryctolagus cuniculus
gatgatagtagtgactgggatgcaaagttcaacttg

SEQ ID NO: 698 - DNA, 336 bp, Oryctolagus cuniculus
gctatccagatgacccagtctccttcctccctgtctgcatctgtaggagacagagtcaccatcacttgccaggccagtcagagcattaacaatgagttatcctggtatcagcagaaaccagggaaagcccctaagctcctgatctatagggcatccactctggcatctggggtcccatcaaggttcagcggcagtggatctgggacagacttcactctcaccatcagcagcctgcagcctgatgattttgcaacttattactgccaacagggttatagtctgaggaacattgataatgctttcggcggagggaccaaggtggaaatcaaacgtacg

SEQ ID NO: 699 - protein, 112 aa, Oryctolagus cuniculus
AIQMTQSPSSLSASVGDRVTITCQASQSINNELSWYQQKPGKAPKLLIYRASTLASGVPSRFSGSGSGTDFTLTISSLQPDDFATYYCQQGYSLRNIDNAFGGGTKVEIKRT

SEQ ID NO: 700 - DNA, 360 bp, Oryctolagus cuniculus
gaggtgcagctggtggagtctgggggaggcttggtccagcctggggggtccctgagactctcctgtgcagcctctggattctccctcagtaactactacgtgacctgggtccgtcaggctccagggaaggggctggagtgggtcggcatcatctatggtagtgatgaaaccgcctacgctacctccgctataggccgattcaccatctccagagacaattccaagaacaccctgtatcttcaaatgaacagcctgagagctgaggacactgctgtgtattactgtgctagagatgatagtagtgactgggatgcaaagttcaacttgtggggccaagggaccctcgtcaccgtctcgagc

SEQ ID NO: 701 - DNA, 651 bp, Oryctolagus cuniculus
gctatccagatgacccagtctccttcctccctgtctgcatctgtaggagacagagtcaccatcacttgccaggccagtcagagcattaacaatgagttatcctggtatcagcagaaaccagggaaagcccctaagctcctgatctatagggcatccactctggcatctggggtcccatcaaggttcagcggcagtggatctgggacagacttcactctcaccatcagcagcctgcagcctgatgattttgcaacttattactgccaacagggttatagtctgaggaacattgataatgctttcggcggagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 702 - protein, 217 aa, Oryctolagus cuniculus
AIQMTQSPSSLSASVGDRVTITCQASQSINNELSWYQQKPGKAPKLLIYRASTLASGVPSRFSGSGSGTDFTLTISSLQPDDFATYYCQQGYSLRNIDNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 703 - DNA, 1350 bp, Oryctolagus cuniculus
gaggtgcagctggtggagtctgggggaggcttggtccagcctggggggtccctgagactctcctgtgcagcctctggattctccctcagtaactactacgtgacctgggtccgtcaggctccagggaaggggctggagtgggtcggcatcatctatggtagtgatgaaaccgcctacgctacctccgctataggccgattcaccatctccagagacaattccaagaacaccctgtatcttcaaatgaacagcctgagagctgaggacactgctgtgtattactgtgctagagatgatagtagtgactgggatgcaaagttcaacttgtggggccaagggaccctcgtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 704 - protein, 450 aa, Oryctolagus cuniculus
EVQLVESGGGLVQPGGSLRLSCAASGFSLSNYYVTWVRQAPGKGLEWVGIIYGSDETAYATSAIGRFTISRDNSKNTLYLQMNSLRAEDTAVYYCARDDSSDWDAKFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 705 - DNA, 705 bp, Oryctolagus cuniculus
atgaagtgggtaacctttatttcccttctgtttctctttagcagcgcttattccgctatccagatgacccagtctccttcctccctgtctgcatctgtaggagacagagtcaccatcacttgccaggccagtcagagcattaacaatgagttatcctggtatcagcagaaaccagggaaagcccctaagctcctgatctatagggcatccactctggcatctggggtcccatcaaggttcagcggcagtggatctgggacagacttcactctcaccatcagcagcctgcagcctgatgattttgcaacttattactgccaacagggttatagtctgaggaacattgataatgctttcggcggagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 706 - protein, 235 aa, Oryctolagus cuniculus
MKWVTFISLLFLFSSAYSAIQMTQSPSSLSASVGDRVTITCQASQSINNELSWYQQKPGKAPKLLIYRASTLASGVPSRFSGSGSGTDFTLTISSLQPDDFATYYCQQGYSLRNIDNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 707 - DNA, 1404 bp, Oryctolagus cuniculus
atgaagtgggtaacctttatttcccttctgtttctctttagcagcgcttattccgaggtgcagctggtggagtctgggggaggcttggtccagcctggggggtccctgagactctcctgtgcagcctctggattctccctcagtaactactacgtgacctgggtccgtcaggctccagggaaggggctggagtgggtcggcatcatctatggtagtgatgaaaccgcctacgctacctccgctataggccgattcaccatctccagagacaattccaagaacaccctgtatcttcaaatgaacagcctgagagctgaggacactgctgtgtattactgtgctagagatgatagtagtgactgggatgcaaagttcaacttgtggggccaagggaccctcgtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 708 - protein, 468 aa, Oryctolagus cuniculus
MKWVTFISLLFLFSSAYSEVQLVESGGGLVQPGGSLRLSCAASGFSLSNYYVTWVRQAPGKGLEWVGIIYGSDETAYATSAIGRFTISRDNSKNTLYLQMNSLRAEDTAVYYCARDDSSDWDAKFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 709 - protein, 111 aa, Oryctolagus cuniculus
AIQMTQSPSSLSASVGDRVTITCQASQSINNELSWYQQKPGKAPKLLIYRASTLASGVPSRFSGSGSGTDFTLTISSLQPDDFATYYCQQGYSLRNIDNAFGGGTKVEIKR

SEQ ID NO: 710 - protein, 11 aa, Homo sapiens
RASQGIRNDLG

SEQ ID NO: 711 - protein, 11 aa, Homo sapiens
RASQGISNYLA

SEQ ID NO: 712 - protein, 11 aa, Homo sapiens
RASQSISSWLA

SEQ ID NO: 713 - protein, 7 aa, Homo sapiens
AASSLQS

SEQ ID NO: 714 - protein, 7 aa, Homo sapiens
AASTLQS

SEQ ID NO: 715 - protein, 7 aa, Homo sapiens
KASSLES

SEQ ID NO: 716 - protein, 5 aa, Homo sapiens
SNYMS

SEQ ID NO: 717 - protein, 16 aa, Homo sapiens
VIYSGGSTYYADSVKG

SEQ ID NO: 718 - protein, 17 aa, Homo sapiens
VIYSGGSSTYYADSVKG

SEQ ID NO: 719 - protein, 330 aa, Artificial Sequence
  Gamma-1 constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 720 - DNA, 297 bp, Oryctolagus cuniculus
atccagatgacccagtctccttcctccctgtctgcatctgtaggagacagagtcaccatcacttgccaggccagtcagagcattaacaatgagttatcctggtatcagcagaaaccagggaaagcccctaagctcctgatctatagggcatccactctggcatctggggtcccatcaaggttcagcggcagtggatctgggacagacttcactctcaccatcagcagcctgcagcctgatgattttgcaacttattactgccaacagggttatagtctgaggaacattgataatgct

SEQ ID NO: 721 - DNA, 333 bp, Oryctolagus cuniculus
gcctatgatatgacccagactccagcctcggtgtctgcagctgtgggaggcacagtcaccatcaagtgccaggccagtcagagcattaacaatgaattatcctggtatcagcagaaaccagggcagcgtcccaagctcctgatctatagggcatccactctggcatctggggtctcatcgcggttcaaaggcagtggatctgggacagagttcactctcaccatcagcgacctggagtgtgccgatgctgccacttactactgtcaacagggttatagtctgaggaatattgataatgctttcggcggagggaccgaggtggtggtcaaacgt

SEQ ID NO: 722 - DNA, 648 bp, Oryctolagus cuniculus
atccagatgacccagtctccttcctccctgtctgcatctgtaggagacagagtcaccatcacttgccaggccagtcagagcattaacaatgagttatcctggtatcagcagaaaccagggaaagcccctaagctcctgatctatagggcatccactctggcatctggggtcccatcaaggttcagcggcagtggatctgggacagacttcactctcaccatcagcagcctgcagcctgatgattttgcaacttattactgccaacagggttatagtctgaggaacattgataatgctttcggcggagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 723 - DNA, 333 bp, Oryctolagus cuniculus
gctatccagatgacccagtctccttcctccctgtctgcatctgtaggagacagagtcaccatcacttgccaggccagtcagagcattaacaatgagttatcctggtatcagcagaaaccagggaaagcccctaagctcctgatctatagggcatccactctggcatctggggtcccatcaaggttcagcggcagtggatctgggacagacttcactctcaccatcagcagcctgcagcctgatgattttgcaacttattactgccaacagggttatagtctgaggaacattgataatgctttcggcggagggaccaaggtggaaatcaaacgt

SEQ ID NO: 724 - DNA, 327 bp, Oryctolagus cuniculus
gaggtgcagctggtggagtctgggggaggcttggtccagcctggggggtccctgagactctcctgtgcagcctctggattctccctcagtaactactacgtgacctgggtccgtcaggctccagggaaggggctggagtgggtcggcatcatctatggtagtgatgaaaccgcctacgctacctccgctataggccgattcaccatctccagagacaattccaagaacaccctgtatcttcaaatgaacagcctgagagctgaggacactgctgtgtattactgtgctagagatgatagtagtgactgggatgcaaagttcaacttg

SEQ ID NO: 725 - DNA, 351 bp, Oryctolagus cuniculus
cagtcgctggaggagtccgggggtcgcctggtcacgcctgggacacccctgacactcacctgcacagcctctggattctccctcagtaactactacgtgacctgggtccgccaggctccagggaaggggctggaatggatcggaatcatttatggtagtgatgaaacggcctacgcgacctgggcgataggccgattcaccatctccaaaacctcgaccacggtggatctgaaaatgaccagtctgacagccgcggacacggccacctatttctgtgccagagatgatagtagtgactgggatgcaaaatttaacttgtggggccaaggcaccctggtcaccgtctcgagc

SEQ ID NO: 726 - protein, 224 aa, Homo sapiens
MEKLLCFLVLTSLSHAFGQTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

SEQ ID NO: 727 - protein, 468 aa, Homo sapiens
MLAVGCALLAALLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQDSSSVPLPTFLVAGGSLAFGTLLCIAIVLRFKKTWKLRALKEGKTSMHPPYSLGQLVPERPRPTPVLVPLISPPVSPSSLGSDNTSSHNRPDARDPRSPYDISNTDYFFPR

SEQ ID NO: 728 - protein, 918 aa, Homo sapiens [sequence omitted]

SEQ ID NO: 729 - protein, 111 aa, Oryctolagus cuniculus
AYDMTQTPASVEVAVGGTVTINCQASETIYSWLSWYQQKPGQPPKLLIYQASDLASGVPSRFSGSGAGTEYTLTISGVQCDDAATYYCQQGYSGSNVDNVFGGGTEVVVKR

SEQ ID NO: 730 - protein, 88 aa, Homo sapiens; identical to SEQ ID NO: 650

SEQ ID NO: 731 - protein, 88 aa, Homo sapiens
DIQMTQSPSTLSASVGDRVTITCRASQSISSWLAWYQQKPGKAPKLLIYDASSLESGVPSRFSGSGSGTEFTLTISSLQPDDFATYYC

SEQ ID NO: 732 - protein, 88 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYC

SEQ ID NO: 733 - protein, 111 aa, Artificial Sequence
  Humanized antibody
DIQMTQSPSTLSASVGDRVTITCQASETIYSWLSWYQQKPGKAPKLLIYQASDLASGVPSRFSGSGSGTEFTLTISSLQPDDFATYYCQQGYSGSNVDNVFGGGTKVEIKR

SEQ ID NO: 734 - protein, 11 aa, Homo sapiens
FGGGTKVEIKR

SEQ ID NO: 735 - protein, 118 aa, Oryctolagus cuniculus
QEQLKESGGRLVTPGTPLTLTCTASGFSLNDHAMGWVRQAPGKGLEYIGFINSGGSARYASWAEGRFTISRTSTTVDLKMTSLTTEDTATYFCVRGGAVWSIHSFDPWGPGTLVTVSS

SEQ ID NO: 736 - protein, 98 aa, Homo sapiens
QVQLVESGGGLVQPGGSLRLSCSASGFTFSSYAMHWVRQAPGKGLEYVSAISSNGGSTYYADSVKGRFTISRDNSKNTLYLQMNSLRAEDTAVYYCAR

SEQ ID NO: 737 - protein, 97 aa, Homo sapiens; identical to SEQ ID NO: 653

SEQ ID NO: 738 - protein, 97 aa, Homo sapiens
EVQLVETGGGLIQPGGSLRLSCAASGFTVSSNYMSWVRQAPGKGLEWVSVIYSGGSTYYADSVKGRFTISRDNSKNTLYLQMNSLRAEDTAVYYCAR

SEQ ID NO: 739 - protein, 120 aa, Artificial Sequence
  Humanized antibody
QVQLVESGGGLVQPGGSLRLSCSASGFSLNDHAMGWVRQAPGKGLEYVGFINSGGSARYASSAEGRFTISRDNSKNTLYLQMNSLRAEDTAVYYCARGGAVWSIHSFDPWGQGTLVTVSS

SEQ ID NO: 740 - protein, 11 aa, Homo sapiens
WGQGTLVTVSS

SEQ ID NO: 741 - DNA, 32 bp, Artificial Sequence
  PCR Primer
agcgcttattccgctatccagatgacccagtc

SEQ ID NO: 742 - DNA, 22 bp, Artificial Sequence
  PCR Primer
cgtacgtttgatttccaccttg

SEQ ID NO: 743 - DNA, 32 bp, Artificial Sequence
  PCR Primer
agcgcttattccgaggtgcagctggtggagtc

SEQ ID NO: 744 - DNA, 20 bp, Artificial Sequence
  PCR Primer
ctcgagacggtgacgagggt

SEQ ID NO: 745 - protein, 450 aa, Artificial Sequence; identical to SEQ ID NO: 704
  Heavy Chain

SEQ ID NO: 746 - protein, 217 aa, Artificial Sequence; identical to SEQ ID NO: 702
  Light Chain